SEQUENCE LISTING (95 sequences)

SEQ ID NO: 1 - protein, 10 aa, Artificial
  CA170_1519 CDRH1
GFTFSNYGMV

SEQ ID NO: 2 - protein, 17 aa, Artificial
  CA170_1519 CDRH2
YIDSDGDNTYYRDSVKG

SEQ ID NO: 3 - protein, 8 aa, Artificial
  CA170_1519 CDRH3
GIVRPFLY

SEQ ID NO: 4 - protein, 16 aa, Artificial
  CA170_1519 CDRL1
KSSQSLVGASGKTYLY

SEQ ID NO: 5 - protein, 7 aa, Artificial
  CA170_1519 CDRL2
LVSTLDS

SEQ ID NO: 6 - protein, 9 aa, Artificial
  CA170_1519 CDRL3
LQGTHFPHT

SEQ ID NO: 7 - protein, 112 aa, Artificial
  Rat Ab 1519 VL region
DVVMTQTPLSLSVALGQPASISCKSSQSLVGASGKTYLYWLFQRSGQSPKRLIYLVSTLDSGIPDRFSGSGAETDFTLKIRRVEADDLGVYYCLQGTHFPHTFGAGTKLELK

SEQ ID NO: 8 - DNA, 336 bp, Artificial
  Rat Ab 1519 VL region
gatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaattgaaa

SEQ ID NO: 9 - protein, 132 aa, Artificial
  Rat Ab 1519 VL region with signal sequence
MMSPAQFLFLLMLWIQGTSGDVVMTQTPLSLSVALGQPASISCKSSQSLVGASGKTYLYWLFQRSGQSPKRLIYLVSTLDSGIPDRFSGSGAETDFTLKIRRVEADDLGVYYCLQGTHFPHTFGAGTKLELK

SEQ ID NO: 10 - DNA, 396 bp, Artificial
  Rat Ab 1519 VL region with signal sequence
atgatgagtcctgcccagttcctgtttctgctgatgctctggattcagggaaccagtggtgatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaattgaaa

SEQ ID NO: 11 - protein, 116 aa, Artificial
  Rat Ab 1519 VH region
EVPLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGTTVTVS

SEQ ID NO: 12 - DNA, 348 bp, Artificial
  Rat Ab 1519 VH region
gaggtgccgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggaaccacggtcaccgtctcg

SEQ ID NO: 13 - protein, 135 aa, Artificial
  Rat Ab 1519 VH region with signal sequence
MDISLSLAFLVLFIKGVRCEVPLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGTTVTVS

SEQ ID NO: 14 - DNA, 405 bp, Artificial
  Rat Ab 1519 VH region with signal sequence
atggacatcagtctcagcttggctttccttgtccttttcataaaaggtgtccggtgtgaggtgccgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggaaccacggtcaccgtctcg

SEQ ID NO: 15 - protein, 112 aa, Artificial [sequence omitted]

SEQ ID NO: 16 - DNA, 336 bp, Artificial
  1519 gL20 V-region (E. coli expression)
gatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaa

SEQ ID NO: 17 - DNA, 336 bp, Artificial
  1519 gL20 V-region (mammalian expression)
gatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaa

SEQ ID NO: 18 - protein, 133 aa, Artificial
  1519 gL20 V-region with signal sequence (E. coli expression)
MKKTAIAIAVALAGFATVAQADIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIK

SEQ ID NO: 19 - DNA, 399 bp, Artificial
  1519 gL20 V-region with signal sequence (E. coli expression)
atgaaaaagacagctatcgcaattgcagtggccttggctggtttcgctaccgtagcgcaagctgatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaa

SEQ ID NO: 20 - protein, 132 aa, Artificial
  1519 gL20 V-region with signal sequence (mammalian expression)
MSVPTQVLGLLLLWLTDARCDIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIK

SEQ ID NO: 21 - DNA, 396 bp, Artificial
  1519 gL20 V-region with signal sequence (mammalian expression)
atgtctgtccccacccaagtcctcggactcctgctactctggcttacagatgccagatgcgatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaa

SEQ ID NO: 22 - protein, 219 aa, Artificial
  1519 gL20 light chain (V + constant)
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 657 bp, Artificial
  1519 gL20 light chain (V + constant, E. coli expression)
gatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcaccagtaacaaaaagttttaatagaggggagtgt

SEQ ID NO: 24 - DNA, 657 bp, Artificial
  1519 gL20 light chain (V + constant, mammalian expression)
gatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 25 - protein, 240 aa, Artificial
  1519 gL20 light chain with signal sequence (E. coli expression)
MKKTAIAIAVALAGFATVAQADIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 720 bp, Artificial
  1519 gL20 light chain with signal sequence (E. coli expression)
atgaaaaagacagctatcgcaattgcagtggccttggctggtttcgctaccgtagcgcaagctgatatccagatgacccagagtccaagcagtctctccgccagcgtaggcgatcgtgtgactattacctgtaaaagctcccagtccctggtgggtgcaagcggcaaaacctacctgtactggctcttccagaaaccgggcaaagctccgaaacgcctgatctatctggtgtctaccctggatagcggtattccgtctcgtttctccggtagcggtagcggtaccgaattcacgctgaccattagctccctccagccggaggactttgctacctattactgcctccagggcactcattttccgcacactttcggccagggtaccaaactggaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcaccagtaacaaaaagttttaatagaggggagtgt

SEQ ID NO: 27 - protein, 239 aa, Artificial
  1519 gL20 light chain with signal sequence (mammalian expression)
MSVPTQVLGLLLLWLTDARCDIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 717 bp, Artificial
  1519 gL20 light chain with signal sequence (mammalian expression)
atgtctgtccccacccaagtcctcggactcctgctactctggcttacagatgccagatgcgatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 29 - protein, 116 aa, Artificial
  1519 gH20 V-region
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVS

SEQ ID NO: 30 - DNA, 348 bp, Artificial
  1519 gH20 V-region (E. coli expression)
gaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcg

SEQ ID NO: 31 - DNA, 348 bp, Artificial
  1519 gH20 V-region (mammalian expression)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcg

SEQ ID NO: 32 - protein, 137 aa, Artificial
  1519 gH20 V-region (E. coli expression)
MKKTAIAIAVALAGFATVAQAEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVS

SEQ ID NO: 33 - DNA, 411 bp, Artificial
  1519 gH20 V-region (E. coli expression)
atgaagaagactgctatagcaattgcagtggcgctagctggtttcgccaccgtggcgcaagctgaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcg

SEQ ID NO: 34 - protein, 135 aa, Artificial
  1519 gH20 V-region (mammalian expression)
MEWSWVFLFFLSVTTGVHSEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVS

SEQ ID NO: 35 - DNA, 405 bp, Artificial
  1519 gH20 V-region with signal sequence (mammalian expression)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcg

SEQ ID NO: 36 - protein, 228 aa, Artificial
  1519gH20 Fab′ heavy chain (V + human gamma-1 CH1 + hinge)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 37 - DNA, 684 bp, Artificial
  1519gH20 Fab′ heavy chain (V + human gamma-1 CH1 + hinge, E.coli expression)
gaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 38 - DNA, 684 bp, Artificial
  1519gH20 Fab′ heavy chain (V + human gamma-1 CH1 + hinge, mammalian expression)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 39 - protein, 249 aa, Artificial
  1519 gH20 Fab′ heavy chain with signal sequence (E. coli expression)
MKKTAIAIAVALAGFATVAQAEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 40 - DNA, 747 bp, Artificial
  1519 gH20 Fab′ heavy chain with signal sequence (E. coli expression)
atgaagaagactgctatagcaattgcagtggcgctagctggtttcgccaccgtggcgcaagctgaggttccgctggtcgagtctggaggcgggcttgtccagcctggagggagcctgcgtctctcttgtgcagtatctggcttcacgttctccaactacggtatggtgtgggttcgtcaggctccaggtaaaggtctggaatgggtggcgtatattgactccgacggcgacaacacctactatcgcgactctgtgaaaggtcgcttcaccatttcccgcgataacgccaaatccagcctgtacctgcagatgaacagcctgcgtgctgaagatactgcggtgtactattgcaccactggcatcgtgcgtccgtttctgtattggggtcagggtaccctcgttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 41 - protein, 247 aa, Artificial
  1519 gH20 Fab′ heavy chain with signal sequence (mammalian expression)
MEWSWVFLFFLSVTTGVHSEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 42 - DNA, 741 bp, Artificial
  1519 gH20 Fab′ heavy chain with signal sequence (mammalian expression)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcgccgcg

SEQ ID NO: 43 - protein, 444 aa, Artificial
  1519gH20 IgG4 heavy chain (V + human gamma-4P constant)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - DNA, 1939 bp, Artificial
  1519gH20 IgG4 heavy chain (V + human gamma-4P constant with exons)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 45 - DNA, 1996 bp, Artificial
  1519gH20 IgG4 heavy chain (V + human gamma-4P constant) with signal sequence
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccaccatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 46 - protein, 347 aa, Artificial
  1519gL20 FabFv light chain
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 47 - DNA, 1041 bp, Artificial
  1519gL20 FabFv light chain
gatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctgagcagcaccctgacgctgtctaaagcagactacgagaaacacaaagtgtacgcctgcgaagtcacccatcagggcctgagctcaccagtaacaaaaagttttaatagaggggagtgtagcggtggcggtggcagtggtgggggaggctccggaggtggcggttcagacatacaaatgacccagagtccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaacgtacc

SEQ ID NO: 48 - protein, 367 aa, Artificial
  1519gL20 FabFv light chain with signal sequence
MSVPTQVLGLLLLWLTDARCDIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 49 - DNA, 1101 bp, Artificial
  1519gL20 FabFv light chain with signal sequence
atgtctgtccccacccaagtcctcggactcctgctactctggcttacagatgccagatgcgatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctgagcagcaccctgacgctgtctaaagcagactacgagaaacacaaagtgtacgcctgcgaagtcacccatcagggcctgagctcaccagtaacaaaaagttttaatagaggggagtgtagcggtggcggtggcagtggtgggggaggctccggaggtggcggttcagacatacaaatgacccagagtccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaacgtacc

SEQ ID NO: 50 - protein, 357 aa, Artificial
  1519gH20 FabFv heavy chain
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGTGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 51 - DNA, 1071 bp, Artificial
  1519gH20 FabFv heavy chain
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagtggaggtgggggctcaggtggaggcgggaccggtggaggtggcagcgaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagt

SEQ ID NO: 52 - protein, 376 aa, Artificial
  1519gH20 FabFv heavy chain with signal sequence
MEWSWVFLFFLSVTTGVHSEVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGTGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 53 - DNA, 1128 bp, Artificial
  1519gH20 FabFv heavy chain with signal sequence
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagtggaggtgggggctcaggtggaggcgggaccggtggaggtggcagcgaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagt

SEQ ID NO: 54 - protein, 107 aa, Artificial
  Human VK1 2-1-(1) A30 JK2 acceptor framework
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 55 - DNA, 321 bp, Artificial
  Human VK1 2-1-(1) A30 JK2 acceptor framework
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccttacacttttggccaggggaccaagctggagatcaaa

SEQ ID NO: 56 - protein, 112 aa, Artificial
  Human VH3 1-3 3-07 JH4 acceptor framework
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARYFDYWGQGTLVTVS

SEQ ID NO: 57 - DNA, 336 bp, Artificial
  Human VH3 1-3 3-07 JH4 acceptor framework
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagatactttgactactggggccagggaaccctggtcaccgtctcc

SEQ ID NO: 58 - protein, 112 aa, Artificial
  Rat Ab 1548 VL region
DVVMTQTPLSLSVALGQPASISCKSSQSLVGASGKTYLYWLFQRSGQSPKRLIYLVSTLDSGIPDRFSGSGAETDFTLKIRRVEADDLGVYYCLQGTHFPHTFGAGTKLEIK

SEQ ID NO: 59 - DNA, 336 bp, Artificial
  Rat Ab 1548 VL region
gatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaaataaaa

SEQ ID NO: 60 - protein, 116 aa, Artificial
  Rat Ab 1548 VH region
EVPLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGVMVTVS

SEQ ID NO: 61 - DNA, 348 bp, Artificial
  Rat Ab 1548 VH region
gaggtgccgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggagtcatggtcacagtctcg

SEQ ID NO: 62 - protein, 112 aa, Artificial
  Rat Ab 1644 VL region
DVVMTQTPLSLSVAIGQPASISCKSSQSLVGASGKTYLYWLFQRSGQSPKRLIYLVSTLDSGIPDRFSGSGAETDFTLKIRRVEADDLGVYYCLQGTHFPHTFGAGTKLELK

SEQ ID NO: 63 - DNA, 336 bp, Artificial
  Rat Ab 1644 VL region
gatgttgtgatgacccagactccactgtctttgtcggttgccattggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaactgaaa

SEQ ID NO: 64 - protein, 116 aa, Artificial
  Rat Ab 1644 VH region
EVPLVESGGGSVQPGRSTKLSCVVSGFTFSNYGMVWVRQAPKKGLEWVAYIGSDGDNIYYRDSVKGRFTISRNNAKSTLYLQMDSLRSEDTATYYCTTGIVRPFLYWGQGTTVTVS

SEQ ID NO: 65 - DNA, 348 bp, Artificial
  Rat Ab 1644 VH region
gaggtgccgctggtggagtctgggggcggctcagtgcagcctgggaggtccacgaaactctcctgtgtagtctcaggattcactttcagtaactatggcatggtctgggtccgccaggctccaaagaagggtctggagtgggtcgcatatattggttctgatggtgataatatttactaccgagattccgtgaagggtcgattcactatctccagaaataatgcaaaaagcaccctatatttgcaaatggacagtctgaggtctgaggacacggccacttattactgtacaacagggattgtccggccctttctctactggggccaaggaaccacggtcaccgtctcg

SEQ ID NO: 66 - protein, 112 aa, Artificial; identical to SEQ ID NO: 7
  Rat Ab 1496 VK region

SEQ ID NO: 67 - DNA, 336 bp, Artificial
  Rat Ab 1496 VK region
gatgttgtgatgacccagactccactgtctttgtcggttgcccttggacaaccagcctccatctcttgcaagtcaagtcagagcctcgtaggtgctagtggaaagacatatttgtattggttatttcagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaagctggaactgaaa

SEQ ID NO: 68 - protein, 116 aa, Artificial
  Rat Ab 1496 VH region
EVLLVESGGGSVQPGRSMKLSCVVSGFTFSNYGMVWVRQAPKKGLEWVAYIDSDGDNTYYRDSVKGRFTISRNNAKSTLYLQMDSLRSEDTATYYCTTGIVRPFLYWGQGTMVTVS

SEQ ID NO: 69 - DNA, 348 bp, Artificial
  Rat Ab 1496 VH region
gaggtgctgctggtggagtctgggggcggctcagtgcagcctgggaggtccatgaaactctcctgtgtagtctcaggattcactttcagtaattatggcatggtctgggtccgccaggctccaaagaagggtctggagtgggtcgcatatattgattctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctattggggccaaggaaccatggtcaccgtctcg

SEQ ID NO: 70 - protein, 14 aa, Artificial
  framework
YFDYWGQGTLVTVS

SEQ ID NO: 71 - protein, 12 aa, Artificial
  framework
YTFGQGTKLEIK

SEQ ID NO: 72 - protein, 447 aa, Artificial
  1519gH20 IgG1 heavy chain (V + human gamma-1 constant)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 1947 bp, Artificial
  1519gH20 IgG1 heavy chain (V + human gamma-1 constant with exons)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 74 - DNA, 2004 bp, Artificial
  1519gH20 IgG1 heavy chain (V + human gamma-1 constant) with signal sequence
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 75 - DNA, 657 bp, Artificial
  1519 gL20 light chain (V + constant, mammalian expression alternative)
gatatccagatgacccagagcccatctagcttatccgcttccgttggtgatcgcgtgacaattacgtgtaagagctcccaatctctcgtgggtgcaagtggcaagacctatctgtactggctctttcagaagcctggcaaggcaccaaaacggctgatctatctggtgtctacccttgactctgggataccgtcacgattttccggatctgggagcggaactgagttcacactcacgatttcatcgctgcaacccgaggactttgctacctactactgcctgcaaggcactcatttccctcacactttcggccaggggacaaaactcgaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 76 - DNA, 684 bp, Artificial
  1519gH20 Fab′ heavy chain (V + human gamma-1 CH1 + hinge, mammalian expression one base change from SEQ ID NO 38)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcgccgcg

SEQ ID NO: 77 - DNA, 741 bp, Artificial
  1519 gH20 Fab′ heavy chain with signal sequence (mammalian expression one base changed from SEQ ID NO 42)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcgccgcg

SEQ ID NO: 78 - protein, 346 aa, Artificial
  1519gL20 FabFv light chain (alternative sequence to SEQ ID NO 46)
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 79 - DNA, 1038 bp, Artificial
  1519gL20 FabFv light chain (alternative sequence to SEQ ID NO 47)
gacatccagatgacccagtccccctccagcctgtccgcctccgtgggcgacagagtgaccatcacatgcaagtcctcccagtccctggtcggagcctccggcaagacctacctgtactggctgttccagaagcccggcaaggcccccaagcggctgatctacctggtgtctaccctggactccggcatcccctcccggttctccggctctggctctggcaccgagttcaccctgaccatctccagcctgcagcccgaggacttcgccacctactactgtctgcaaggcacccacttcccccacaccttcggccagggcaccaagctggaaatcaagcggaccgtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtggaggtggctctggcggtggtggctccggaggcggaggaagcgacatccagatgacccagagcccttcctctgtaagcgccagtgtcggagacagagtgactattacctgccaaagctccccttcagtctggtccaattttctatcctggtatcagcaaaagcccggaaaggctcctaaattgctgatctacgaagcaagcaaactcaccagcggcgtgcccagcaggttcagcggcagtgggtctggaactgactttaccctgacaatctcctcactccagcccgaggacttcgccacctattactgcggtggaggttacagtagcataagtgatacgacatttggatgcggcactaaagtggaaatcaagcgtacc

SEQ ID NO: 80 - DNA, 1071 bp, Artificial
  1519gH20 FabFv heavy chain (alternative sequence to SEQ ID NO 51)
gaggtgcccctggtggaatctggcggcggactggtgcagcctggcggctccctgagactgtcttgcgccgtgtccggcttcaccttctccaactacggcatggtctgggtccgacaggctcctggcaagggactggaatgggtggcctacatcgactccgacggcgacaacacctactaccgggactccgtgaagggccggttcaccatctcccgggacaacgccaagtcctccctgtacctgcagatgaactccctgcgggccgaggacaccgccgtgtactactgcaccaccggcatcgtgcggccctttctgtactggggccagggcaccctggtcaccgtgtcctctgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctctggactctactccctcagcagcgtggtgaccgtgccctccagcagcttgggcacccagacctacatctgcaacgtgaatcacaagcccagcaacaccaaggtggacaagaaagttgagcccaaatcttgttccggaggtggcggttccggaggtggcggtacaggtggcggtgggtccgaagtccagctgcttgaatccggaggcggactcgtgcagcccggaggcagtcttcgcttgtcctgcgctgtatctggaatcgacctgagcaattacgccatcaactgggtgagacaggcacctgggaaatgcctcgaatggatcggcattatatgggctagtgggacgaccttttatgctacatgggcgaagggtagattcacaatctcacgggataatagtaagaacacagtgtacctgcagatgaactccctgcgagcagaggataccgccgtttactattgtgctcgcactgtcccaggttatagcactgcaccctactttgatctgtgggggcagggcactctggtcaccgtctcgtcc

SEQ ID NO: 81 - protein, 112 aa, Artificial
  Rat Ab 1548 VL region (alternative sequence to SEQ ID NO 58)
DVVMTQTPLSLSVAIGQPASISSKSSQSLVGAGGKTYLYWLLQRSGQSPKRLIYLVSTLDSGIPDRFSGSGAETDFTLKIRRVEADDLGVYYCLQGTHFPHTFGAGTNLEIK

SEQ ID NO: 82 - DNA, 336 bp, Artificial
  Rat Ab 1548 VL region (alternative sequence to SEQ ID NO 59)
gatgttgtgatgacccagactccactgtctttgtcggttgccattggacaaccagcctccatctcttctaagtcaagtcagagcctcgtaggtgctggtggaaagacatatttgtattggttattacagaggtccggccagtctccaaagcgactaatctatctggtgtccacactggactctggaattcctgataggttcagtggcagtggagcagagacagattttactcttaaaatccgcagagtggaagccgatgatttgggagtttattactgcttgcaaggtacacattttcctcacacgtttggagctgggaccaacctggaaataaaa

SEQ ID NO: 83 - protein, 116 aa, Artificial
  Rat Ab 1548 VH region (alternative sequence to SEQ ID NO 60)
EVPLVESGGGSVQPGRSMKLSCVVSGFTFSNYGMVWVRQAPKKGLEWVAYIGSDGDNTYYRDSVKGRFTISRNNAKSTLYLQMDSLRSEDTATYYCTTGIVRPFLYWGQGVMVTVS

SEQ ID NO: 84 - DNA, 348 bp, Artificial
  Rat Ab 1548 VH region (alternative sequence to SEQ IS NO 61)
gaggtgccgctggtggagtctgggggcggctcagtgcagcctgggaggtccatgaaactctcctgtgtagtctcaggattcactttcagtaactatggcatggtctgggtccgccaggctccaaagaagggtctggagtgggtcgcatatattggttctgatggtgataatacttactaccgagattccgtgaagggccgattcactatctccagaaataatgcaaaaagcaccctatatttgcaaatggacagtctgaggtctgaggacacggccacttattactgtacaacagggattgtccggccctttctctactggggccaaggagtcatggtcacagtctcg

SEQ ID NO: 85 - DNA, 1947 bp, Artificial
  1519gH20 IgG1 heavy chain (V + human gamma-1 constant with exons one base change to SEQ ID NO 71)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 86 - DNA, 2004 bp, Artificial
  1519gH20 IgG1 heavy chain (V + human gamma-1 constant) with signal sequence (one base change from SEQ ID NO72)
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagatctgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - protein, 444 aa, Artificial
  1519gH20 IgG4 heavy chain (V + human gamma-4 constant no P mutations)
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - DNA, 1939 bp, Artificial
  1519gH20 IgG4 heavy chain (V + human gamma-4 constant with exons no P mutations)
gaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 89 - DNA, 1996 bp, Artificial
  1519gH20 IgG4 heavy chain (V + human gamma-4 constant) with signal sequence - no P mutation
atggaatggagctgggtctttctcttcttcctgtcagtaactacaggagtccattctgaggtaccacttgtggaaagcggaggaggtcttgtgcagcctggaggaagtttacgtctctcttgtgctgtgtctggcttcaccttctccaattacggaatggtctgggtcagacaagcacctggaaagggtcttgaatgggtggcctatattgactctgacggggacaacacctactatcgggattccgtgaaaggacgcttcacaatctcccgagataacgccaagagctcactgtacctgcagatgaatagcctgagagccgaggatactgccgtgtactattgcacaacgggaatcgttaggccttttctgtactggggacagggcaccttggttactgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgcccagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 90 - DNA, 336 bp, Artificial
  1519 gL20 V-region (mammalian expression alternative to SEQ ID NO 17)
gacatccagatgacccagtccccctccagcctgtccgcctccgtgggcgacagagtgaccatcacatgcaagtcctcccagtccctggtcggagcctccggcaagacctacctgtactggctgttccagaagcccggcaaggcccccaagcggctgatctacctggtgtctaccctggactccggcatcccctcccggttctccggctctggctctggcaccgagttcaccctgaccatctccagcctgcagcccgaggacttcgccacctactactgtctgcaaggcacccacttcccccacaccttcggccagggcaccaagctggaaatcaag

SEQ ID NO: 91 - DNA, 657 bp, Artificial
  1519 gL20 light chain (V + constant, mammalian expression) alternative to SEQ ID NO 24)
gacatccagatgacccagtccccctccagcctgtccgcctccgtgggcgacagagtgaccatcacatgcaagtcctcccagtccctggtcggagcctccggcaagacctacctgtactggctgttccagaagcccggcaaggcccccaagcggctgatctacctggtgtctaccctggactccggcatcccctcccggttctccggctctggctctggcaccgagttcaccctgaccatctccagcctgcagcccgaggacttcgccacctactactgtctgcaaggcacccacttcccccacaccttcggccagggcaccaagctggaaatcaagcggaccgtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 92 - DNA, 348 bp, Artificial
  1519 gH20 V-region (mammalian expression alternative to SEQ ID NO 31)
gaggtgcccctggtggaatctggcggcggactggtgcagcctggcggctccctgagactgtcttgcgccgtgtccggcttcaccttctccaactacggcatggtctgggtccgacaggctcctggcaagggactggaatgggtggcctacatcgactccgacggcgacaacacctactaccgggactccgtgaagggccggttcaccatctcccgggacaacgccaagtcctccctgtacctgcagatgaactccctgcgggccgaggacaccgccgtgtactactgcaccaccggcatcgtgcggccctttctgtactggggccagggcaccctggtcaccgtgtcc

SEQ ID NO: 93 - DNA, 1332 bp, Artificial
  1519gH20 IgG4 heavy chain (V + human gamma-4P constant alternative to SEQ ID NO 44)
gaggtgcccctggtggaatctggcggcggactggtgcagcctggcggctccctgagactgtcttgcgccgtgtccggcttcaccttctccaactacggcatggtctgggtccgacaggctcctggcaagggactggaatgggtggcctacatcgactccgacggcgacaacacctactaccgggactccgtgaagggccggttcaccatctcccgggacaacgccaagtcctccctgtacctgcagatgaactccctgcgggccgaggacaccgccgtgtactactgcaccaccggcatcgtgcggccctttctgtactggggccagggcaccctggtcaccgtgtcctctgcctccaccaagggcccctccgtgttccctctggccccttgctcccggtccacctccgagtctaccgccgctctgggctgcctggtcaaggactacttccccgagcccgtgacagtgtcctggaactctggcgccctgacctccggcgtgcacaccttccctgccgtgctgcagtcctccggcctgtactccctgtcctccgtcgtgaccgtgccctcctccagcctgggcaccaagacctacacctgtaacgtggaccacaagccctccaacaccaaggtggacaagcgggtggaatctaagtacggccctccctgccccccctgccctgcccctgaatttctgggcggaccttccgtgttcctgttccccccaaagcccaaggacaccctgatgatctcccggacccccgaagtgacctgcgtggtggtggacgtgtcccaggaagatcccgaggtccagttcaattggtacgtggacggcgtggaagtgcacaatgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtcaagggcttctacccctccgacattgccgtggaatgggagtccaacggccagcccgagaacaactacaagaccaccccccctgtgctggacagcgacggctccttcttcctgtactctcggctgaccgtggacaagtcccggtggcaggaaggcaacgtcttctcctgctccgtgatgcacgaggccctgcacaaccactacacccagaagtccctgtccctgagcctgggcaag

SEQ ID NO: 94 - protein, 274 aa, Artificial
  FcRn alpha chain extracellular sequence
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 95 - protein, 99 aa, Homo sapiens
IQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

(19) United States
(12) Patent Application Publication
(10) Pub. No.: US 2020/0140548 A1
(43) Pub. Date: May 7, 2020

(54) METHOD FOR THE TREATMENT OF IMMUNE THROMBOCYTOPENIA

(71) Applicant: UCB BIOPHARMA SRL, Brussels (BE)

(72) Inventors: Rocio Lledo-Garcia, Slough (GB); Grant Langdon, Hampshire (GB); Claus Peter Kiessling, Monheim Am Rhein (DE); Rose Gunter Snipes, Wilmimgton, DE (US); Ute Massow, Monheim Am Rhein (DE)

(21) Appl. No.: 16/622,658

(22) PCT Filed: Jun. 15, 2018
(86) PCT No.: PCT/EP2018/065947

§ 371 Date: Dec. 13, 2019

(30) Foreign Application Priority Data
Jun. 15, 2017 (GB) 1709554.8
Nov. 10, 2017 (GB) 1718589.3

(51) Int. Cl. C07K 16/28; A61K 9/00; A61P 7/02

(52) U.S. Cl. CPC C07K 16/283; A61K 9/0019; A61P 7/02; A61K 2039/545; C07K 2317/24; C07K 2317/55; C07K 2317/624; C07K 2317/565

(57) ABSTRACT

The disclosure relates to a method of treating or preventing immune (idiopathic) thrombocytopenic purpura (ITP) in a human in need thereof, the method comprising administering to the human in the range of 1 to 5 doses of an anti-FcRn antibody or antigen binding fragment thereof over a treatment period of 1 to 12 weeks, wherein the aggregate dose in the treatment period is in the range of 1 to 30 mg per kg.

The disclosure relates to a method of treating immune (idiopathic) thrombocytopenia (ITP) using antibodies specific to FcRn.

The neonatal MHC-class-I-like FcRn recycles immunoglobulin and albumin from most cells and transports it bi-directionally across epithelial barriers to affect systemic and mucosal immunity. It was shown that FcRn rescues both IgG and albumin from intracellular lysosomal degradation by recycling it from the sorting endosome to the cell surface (Anderson et al, 2006). With respect to IgG, this is achieved by interaction of IgG with the receptor, FcRn. Thus, in effect FcRn salvages IgG, saving it from degradation and returning it to circulation. Albumin is similarly recycled by FcRn, though via a different binding site on the FcRn molecule. It has been shown that knockout or blockade of FcRn removes this recycling resulting in endosomal catabolism of IgG and a marked reduction of IgG concentrations in both the vascular and extravascular (tissue) compartments. In effect, blockade of FcRn accelerates removal of endogenous IgG and, if the albumin binding site is also blocked, potentially of albumin.

Idiopathic or Immune thrombocytopenia (ITP), also known as immune (idiopathic) thrombocytopenic purpura (ITP), is a clinical disorder in which thrombocytopenia manifests as a bleeding tendency, purpura, or petechiae. Autoantibodies against platelet antigens are considered to be a hallmark of ITP. Production of pathogenic IgG autoantibodies by plasma cells is accepted as the central underlying pathophysiological mechanism in a number of IgG-mediated autoimmune diseases, which includes ITP. In some patients, antibodies recognize antigens derived from a single glycoprotein, whereas in others, antibodies recognize multiple glycoproteins. The spleen is the key organ in the pathophysiology of ITP, not only because platelet autoantibodies are formed in the white pulp, but also because mononuclear macrophages in the red pulp destroy immunoglobulin-coated platelets (Sandler, Semin Hematol. 2000; 37(1 Suppl 1):10-2.).

Recently, new definitions for the phases of the disease were introduced (Rodeghiero et al, 2009 Blood. 2009; 113(11):2386-93). Based on the time from diagnosis, the first phase (up to 3 months) is the “newly-diagnosed ITP,” phase 2 (>3 months up to 12 months) is the “persistent phase,” and after 12 months the “chronic phase” starts. These phases also reflect the line of treatment.

The major goal for treatment of ITP is to achieve a platelet count that prevents major bleeding rather than correcting the platelet count to normal levels. The management of ITP should be tailored to the individual patient and it is rarely indicated in those with platelet counts above 50×10⁹/L in the absence of bleeding, trauma, surgery, or high risk factors (eg, patients on anticoagulation therapy) (EMA/CHMP/153191/2013, 2014). However, there is general agreement that adults with a count of <30×10⁹/L with bleeding at diagnosis require treatment.

The first line of treatment for newly-diagnosed ITP is generally agreed and based on the use of corticosteroids and intravenous immunoglobulin (IVIg). Patients who fail to respond or who relapse face the options of treatment with second line drug therapy or splenectomy but there is no clear evidence to support the best approach. Splenectomy can provide long-term efficacy in around 60% of cases and recent guidelines suggest considering a splenectomy after 12 months. Splenectomy is an invasive procedure associated with acute complications due to thrombocytopenia (like bleeding events) and long-term complications from loss of splenic functions. Asplenic subjects are at increased risk of life-threatening infections. Splenectomy may increase morbidity from venous thromboembolism or atherosclerosis (Ghanima et al, 2012).

Second line drug therapies include high-dose dexamethasone or methylprednisolone; high-dose IVIg or anti-D Ig; vinca alkaloids; dapsone and danazol; the immunosuppressants cyclophosphamide, azathioprine, and cyclosporine; or mycophenolate mofetil and Helicobacter pylori eradication if applicable. The anti-CD20 monoclonal antibody rituximab—even if not licensed for the treatment of ITP—and the thrombopoietin-receptor (TPO-R) agonists are considered as second line as well as third line options.

Treatment-related morbidity is also a significant contributing factor; long-term courses of corticosteroids, other immunosuppressive medications, or splenectomy may be required to maintain a platelet count in a safe range in patients with chronic treatment-resistant ITP and morbidity and mortality can be related to treatment, reflecting the complications of therapy with corticosteroids or splenectomy. There thus remains a considerable unmet medical need for new therapeutic options in the treatment of ITP.

Accordingly agents that block or reduce the binding of IgG to FcRn may be useful in the treatment or prevention of ITP by removal of pathogenic IgG. Anti-FcRn antibodies have been described previously in WO2009/131702, WO2007/087289, WO2006/118772, WO2014/019727, WO2015/071330, WO2015/167293 and WO2016/123521.

UCB7665 (rozanolixizumab) is a humanized anti-FcRn monoclonal antibody that has been specifically designed to inhibit IgG binding to FcRn without inhibiting albumin binding to FcRn (described herein and in WO2014/019727). UCB7665 is being developed as an inhibitor of FcRn activity with the aim to reduce the concentration of pathogenic IgG in patients with ITP. UCB7665 was derived from a rat antibody with specificity for human FcRn by engineering the rat antibody into a humanized IgG4P format. The construct encoding UCB7665 was created by grafting the complementarity-determining region (CDRs) from the parental rat heavy and light chain variable regions onto a human IgG4P and kappa chain genetic background (SEQ ID NO:43 and SEQ ID NO:22 respectively).

### SUMMARY OF THE DISCLOSURE

The present disclosure demonstrates for the first time, the therapeutic efficacy of anti-FcRn antibodies in the treatment of ITP in humans and provides suitable dosage regimens for such treatment.

Thus in one aspect there is provided a method of treating or preventing immune (idiopathic) thrombocytopenia (ITP) in a human in need thereof, the method comprising administering to the human in the range of 1 to 5 doses of an anti-FcRn antibody or antigen binding fragment thereof over a treatment period of 1 to 12 weeks, wherein the aggregrate dose in the treatment period is in the range of 1 to 30 mg per kg.

In one example, the anti-FcRn antibody or antigen binding fragment thereof comprises:
a. a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
b. a light chain or light chain fragment thereof having a variable region, wherein said variable region comprises three CDRs having the sequences given in SEQ ID NO: 4 for CDR L1, SEQ ID NO: 5 for CDR L2 and SEQ ID NO: 6 for CDR L3.

In another aspect, there is provided an anti-FcRn antibody or binding fragment thereof for use in the treatment or prevention of idiopathic thrombocytopenic purpura (ITP) in a human in need thereof, comprising administering to the human in the range of 1 to 5 doses of the antibody or antigen binding fragment thereof over a treatment period of 1 to 12 weeks, wherein the aggregrate dose in the treatment period is in the range of 1 to 30 mg per kg.

In another aspect there is provided an anti-FcRn antibody or antigen binding fragment thereof comprising:

a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
a light chain or light chain fragment thereof having a variable region comprising three CDRs having the sequences given in SEQ ID NO: 4 for CDR L1, SEQ ID NO: 5 for CDR L2 and SEQ ID NO: 6 for CDR L3,
for use in the treatment or prevention of immune (idiopathic) thrombocytopenic purpura (ITP) comprising administering in the range of 1 to 5 doses of the antibody or antigen binding fragment in a treatment period of 1 to 12 weeks,
wherein the aggregrate dose in the treatment period is in the range of 1 to 30 mg per Kg.

In another aspect there is provided the use of an anti-FcRn antibody or binding fragment thereof comprising:
i. a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
ii. a light chain or light chain fragment thereof having a variable region comprising three CDRs having the sequences given in SEQ ID NO: 4 for CDR L1, SEQ ID NO: 5 for CDR L2 and SEQ ID NO: 6 for CDR L3,
for the manufacture of a medicament for the treatment or prevention of immune (idiopathic) thrombocytopenic purpura (ITP) comprising administering in the range of 1 to 5 doses of the antibody or binding fragment in a treatment period of 1 to 12 weeks, wherein the aggregrate dose in the treatment period is in the range of 1 to 30 mg per Kg

Importantly the antibodies of the present invention are able to bind human FcRn at both pH6 and pH7.4 with comparable and high binding affinity. Advantageously therefore the antibodies are able to continue to bind FcRn even within the endosome, thereby maximising the blocking of FcRn binding to IgG.

In one example, the anti-FcRn antibody or binding fragment thereof for use in the present invention binds an epitope of human FcRn which comprises at least one amino acid selected from the group consisting of residues V105, P106, T107, A108 and K109 of SEQ ID NO:94 and at least one residue, for example at least 2, 3, 4, 5, 6, 7, 8, 9 or 10 residues selected from the group consisting of P100, E115, E116, F117, M118, N119, F120, D121, L122, K123, Q124, G128, G129, D130, W131, P132 and E133 of SEQ ID NO:94

In one embodiment the antibodies or binding fragments according to the present disclosure comprise a light chain or light chain fragment having a variable region, for example comprising one, two or three CDRs independently selected from SEQ ID NO: 4, SEQ ID NO: 5 and SEQ ID NO: 6, in particular wherein CDR L1 is SEQ ID NO: 4, CDR L2 is SEQ ID NO: 5 and CDR L3 is SEQ ID NO: 6.

In one embodiment the antibodies or binding fragments according to the present disclosure comprise CDR sequences of SEQ ID NOs: 1 to 6, for example wherein CDR H1 is SEQ ID NO: 1, CDR H2 is SEQ ID NO: 2, CDR H3 is SEQ ID NO: 3, CDR L1 is SEQ ID NO: 4, CDR L2 is SEQ ID NO: 5 and CDR L3 is SEQ ID NO: 6.

The disclosure also extends to a polynucleotide, such as DNA, encoding an antibody or fragment as described herein.

Also provided is a host cell comprising said polynucleotide.

Methods of expressing an antibody or fragment are provided herein as are methods of conjugating an antibody or fragment to a polymer, such as PEG.

The present disclosure also relates to pharmaceutical compositions comprising said antibodies and fragments.

### BRIEF DESCRIPTION OF THE FIGURES

FIG. 1 shows certain amino acid and polynucleotide sequences.

FIG. 2 shows alignments of certain sequences.

FIG. 3 ITP dosing regimens

FIG. 4 ITP Bleeding Assessment Tool (ITP-BAT)

FIG. 5 Mean IgG reduction following UCB7665 administration

FIG. 6 Mean platelet count (responders only) following UCB7665 administration

FIG. 7 Mean IgG reduction following multiple UCB7665 4, 7, 10 and single 15 mg/kg administration

### DETAILS OF THE DISCLOSURE

FcRn as employed herein refers to the non-covalent complex between the human IgG receptor alpha chain, also known as the neonatal Fc receptor, the amino acid sequence of which is in UniProt under number P55899 together with β2 microglobulin (P2M), the amino acid sequence of which is in UniProt under number P61769.

Antibody molecule as employed herein refers to an antibody or binding fragment thereof.

The term ‘antibody’ as used herein generally relates to intact (whole) antibodies i.e. comprising the elements of two heavy chains and two light chains. The antibody may comprise further additional binding domains for example as per the molecule DVD-Ig as disclosed in WO 2007/024715, or the so-called (FabFv)₂Fc described in WO2011/030107. Thus antibody as employed herein includes bi, tri or tetra-valent full length antibodies.

Antigen binding fragments of antibodies include single chain antibodies (i.e. a full length heavy chain and light chain); Fab, modified Fab, Fab′, modified Fab′, F(ab′)2, Fv, Fab-Fv, Fab-dsFv, single domain antibodies (e.g. VH or VL or VHH), scFv, bi, tri or tetra-valent antibodies, Bis-scFv, diabodies, tribodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). The Fab-Fv format was first disclosed in WO2009/040562 and the disulphide stabilised versions thereof, the Fab-dsFv was first disclosed in WO2010/035012. Other antibody fragments for use in the present invention include the Fab and Fab′ fragments described in International patent applications WO2005/003169, WO2005/003170 and WO2005/003171. Multi-valent antibodies may comprise multiple specificities e.g. bispecific or may be monospecific (see for example WO 92/22583 and WO05/113605). One such example of the latter is a Tri-Fab (or TFM) as described in WO92/22583.

A typical Fab′ molecule comprises a heavy and a light chain pair in which the heavy chain comprises a variable region V_{H}, a constant domain C_{H}1 and a natural or modified hinge region and the light chain comprises a variable region V_{L} and a constant domain CL.

In one embodiment there is provided a dimer of a Fab′ according to the present disclosure to create a F(ab′)2 for example dimerisation may be through the hinge.

In one embodiment the antibody or binding fragment thereof comprises a binding domain. A binding domain will generally comprises 6 CDRs, three from a heavy chain and three from a light chain. In one embodiment the CDRs are in a framework and together form a variable region. Thus in one embodiment an antibody or binding fragment comprises a binding domain specific for antigen comprising a light chain variable region and a heavy chain variable region.

It will be appreciated that one or more (for example 1, 2, 3 or 4) amino acid substitutions, additions and/or deletions may be made to the CDRs or other sequences (e.g variable domains) provided by the present invention without significantly altering the ability of the antibody to bind to FcRn. The effect of any amino acid substitutions, additions and/or deletions can be readily tested by one skilled in the art, for example by using the methods described in WO2014/019727 to determine FcRn binding and blocking.

In one example one or more (for example 1, 2, 3 or 4) amino acid substitutions, additions and/or deletions may be made to the framework region employed in the antibody or fragment provided by the present invention and wherein binding affinity to FcRn is retained or increased.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

Antibodies and fragments of the present disclosure block FcRn and may thereby prevent it functioning in the recycling of IgG. Blocking as employed herein refers to physically blocking such as occluding the receptor but will also include where the antibody or fragments binds an epitope that causes, for example a conformational change which means that the natural ligand to the receptor no longer binds. Antibody molecules of the present invention bind to FcRn and thereby decrease or prevent (e.g. inhibit) FcRn binding to an IgG constant region.

In one embodiment the antibody or fragment thereof binds FcRn competitively with respect to IgG.

In one example the antibody or binding fragment thereof functions as a competitive inhibitor of human FcRn binding to human IgG. In one example the antibody or binding fragment thereof binds to the IgG binding site on FcRn. In one example the antibody or binding fragment thereof does not bind β2M.

Antibodies for use in the present disclosure may be obtained using any suitable method known in the art. The FcRn polypeptide/protein including fusion proteins, cells (recombinantly or naturally) expressing the polypeptide (such as activated T cells) can be used to produce antibodies which specifically recognise FcRn. The polypeptide may be the ‘mature’ polypeptide or a biologically active fragment or derivative thereof. The human protein is registered in Swiss-Prot under the number P55899. The extracellular domain of human FcRn alpha chain is provided in SEQ ID NO:94. The sequence of 32M is provided in SEQ ID NO:95.

In one embodiment the antigen is a mutant form of FcRn which is engineered to present FcRn on the surface of a cell, such that there is little or no dynamic processing where the FcRn is internalised in the cell, for example this can be achieved by making a mutation in the cytoplasmic tail of the FcRn alpha chain, wherein di-leucine is mutated to di-alanine as described in Ober et al 2001 Int. Immunol. 13, 1551-1559.

Polypeptides, for use to immunize a host, may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The FcRn polypeptide may in some instances be part of a larger protein such as a fusion protein for example fused to an affinity tag or similar.

Antibodies generated against the FcRn polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by, for example, the methods described by Babcook, J. et al., 1996, Proc. Natl. Acad. Sci. USA 93(15):7843-78481; WO92/02551; WO2004/051268 and International Patent Application number WO2004/106377.

Screening for antibodies can be performed using assays to measure binding to human FcRn and/or assays to measure the ability to block IgG binding to the receptor. An example of a binding assay is an ELISA, in particular, using a fusion protein of human FcRn and human Fc, which is immobilized on plates, and employing a secondary antibody to detect anti-FcRn antibody bound to the fusion protein. Examples of suitable antagonistic and blocking assays are well known in the art and described in WO2014/019727.

Humanised antibodies (which include CDR-grafted antibodies) are antibody molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived. The latter are often referred to as donor residues.

Specific as employed herein is intended to refer to an antibody that only recognises the antigen to which it is specific or an antibody that has significantly higher binding affinity to the antigen to which it is specific compared to binding to antigens to which it is non-specific, for example at least 5, 6, 7, 8, 9, 10 times higher binding affinity. Binding affinity may be measured by techniques such as BIAcore as described herein and in WO2014/019727. In one example the antibody of the present invention does not bind β2 microglobulin (P2M). In one example the antibody of the present invention binds cynomolgus FcRn. In one example the antibody of the present invention does not bind rat or mouse FcRn.

The amino acid sequences and the polynucleotide sequences of certain antibodies according to the present disclosure are provided in the Figures.

Other antibodies useful in the present invention are described in WO2009/131702, WO2007/087289, WO2006/118772, WO2015/071330, WO2015/167293 and WO2016/123521

and are incorporated herein by reference.

In one embodiment the antibody or fragments according to the disclosure are humanised.

As used herein, the term ‘humanised antibody molecule’ refers to an antibody molecule wherein the heavy and/or light chain contains one or more CDRs (including, if desired, one or more modified CDRs) from a donor antibody (e.g. a non-human antibody such as a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions.

Suitably, the humanised antibody according to the present invention has a variable domain comprising human acceptor framework regions as well as one or more of the CDRs provided specifically herein. Thus, provided in one embodiment is blocking humanised antibody which binds human FcRn wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody of the present invention, the acceptor heavy and light chains do not necessarily need to be derived from the same antibody and may, if desired, comprise composite chains having framework regions derived from different chains.

One such suitable framework region for the heavy chain of the humanised antibody of the present invention is derived from the human sub-group VH3 sequence 1-3 3-07 together with JH4 (SEQ ID NO: 56).

Accordingly, in one example there is provided a humanised antibody comprising the sequence given in SEQ ID NO: 1 for CDR-H1, the sequence given in SEQ ID NO: 2 for CDR-H2 and the sequence given in SEQ ID NO: 3 for CDRH3, wherein the heavy chain framework region is derived from the human subgroup VH3 sequence 1-3 3-07 together with JH4.

The sequence of human JH4 is as follows: (YFDY)WGQGTLVTVS (Seq ID No: 70). The YFDY motif is part of CDR-H3 and is not part of framework 4 (Ravetch, J V. et al., 1981, Cell, 27, 583-591).

In one example the heavy chain variable domain of the antibody comprises the sequence given in SEQ ID NO: 29.

A suitable framework region for the light chain of the humanised antibody of the present invention is derived from the human germline sub-group VK1 sequence 2-1-(1) A30 together with JK2 (SEQ ID NO: 54).

Accordingly, in one example there is provided a humanised antibody comprising the sequence given in SEQ ID NO: 4 for CDR-L1, the sequence given in SEQ ID NO: 5 for CDR-L2 and the sequence given in SEQ ID NO: 6 for CDRL3, wherein the light chain framework region is derived from the human subgroup VK1 sequence 2-1-(1) A30 together with JK2.

The JK2 sequence is as follows: (YT)FGQGTKLEIK (Seq ID No: 71). The YT motif is part of CDR-L3 and is not part of framework 4 (Hieter, P A., et al., 1982, J. Biol. Chem., 257, 1516-1522).

In one example the light chain variable domain of the antibody comprises the sequence given in SEQ ID NO: 15.

In a humanised antibody of the present invention, the framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

Thus in one embodiment 1, 2, 3, 4, or 5 residues in the framework are replaced with an alternative amino acid residue.

Accordingly, in one example there is provided a humanised antibody, wherein at least the residues at each of positions 3, 24, 76, 93 and 94 of the variable domain of the heavy chain (Kabat numbering) are donor residues, see for example the sequence given in SEQ ID NO: 29.

In one embodiment residue 3 of the heavy chain variable domain is replaced with an alternative amino acid, for example glutamine.

In one embodiment residue 24 of the heavy chain variable domain is replaced with an alternative amino acid, for example alanine.

In one embodiment residue 76 of the heavy chain variable domain is replaced with an alternative amino acid, for example asparagine.

In one embodiment residue 93 of the heavy chain is replaced with an alternative amino acid, for example alanine.

In one embodiment residue 94 of the heavy chain is replaced with an alternative amino acid, for example arginine.

In one embodiment residue 3 is glutamine, residue 24 is alanine, residue 76 is aspargine, residue 93 is alanine and residue 94 is arginine in the humanised heavy chain variable region according to the present disclosure.

Accordingly, in one example there is provided a humanised antibody, wherein at least the residues at each of positions 36, 37 and 58 of the variable domain of the light chain (Kabat numbering) are donor residues, see for example the sequence given in SEQ ID NO: 15

In one embodiment residue 36 of the light chain variable domain is replaced with an alternative amino acid, for example tyrosine.

In one embodiment residue 37 of the light chain variable domain is replaced with an alternative amino acid, for example glutamine.

In one embodiment residue 58 of the light chain variable domain is replaced with an alternative amino acid, for example valine.

In one embodiment residue 36 is tyrosine, residue 37 is glutamine and residue 58 is valine, in the humanised heavy chain variable region according to the present disclosure.

In one embodiment the disclosure provides an antibody sequence which is 80% similar or identical to a sequence disclosed herein, for example 85%, 90%, 91%, 92%, 93%, 94%, 95% 96%, 97%, 98% or 99% over part or whole of the relevant sequence, for example a variable domain sequence, a CDR sequence or a variable domain sequence, excluding the CDRs. In one embodiment the relevant sequence is SEQ ID NO: 15. In one embodiment the relevant sequence is SEQ ID NO: 29.

In one embodiment, the present invention provides an antibody molecule which binds human FcRn comprising a heavy chain, wherein the variable domain of the heavy chain comprises a sequence having at least 80%, 85%, 90%, 91%, 92%, 93%, 94%, 95% 96%, 97%, 98% or 99% identity or similarity to the sequence given in SEQ ID NO:29.

In one embodiment, the present invention provides an antibody molecule which binds human FcRn comprising a light chain, wherein the variable domain of the light chain comprises a sequence having at least 80%, 85%, 90%, 91%, 92%, 93%, 94%, 95% 96%, 97%, 98% or 99% identity or similarity to the sequence given in SEQ ID NO:15.

In one embodiment the present invention provides an antibody molecule which binds human FcRn wherein the antibody has a heavy chain variable domain which is at least 90%, 91%, 92%, 93%, 94%, 95% 96%, 97%, 98% or 99% similar or identical to the sequence given in SEQ ID NO:29 but wherein the antibody molecule has the sequence given in SEQ ID NO: 1 for CDR-H1, the sequence given in SEQ ID NO: 2 for CDR-H2 and the sequence given in SEQ ID NO: 3 for CDR-H3.

In one embodiment the present invention provides an antibody molecule which binds human FcRn wherein the antibody has a light chain variable domain which is at least 90%, 91%, 92%, 93%, 94%, 95% 96%, 97%, 98% or 99% similar or identical to the sequence given in SEQ ID NO: 15 but wherein the antibody molecule has the sequence given in SEQ ID NO: 4 for CDR-L1, the sequence given in SEQ ID NO: 5 for CDR-L2 and the sequence given in SEQ ID NO:6 for CDR-L3.

In one embodiment the present invention provides an antibody molecule which binds human FcRn wherein the antibody has a heavy chain variable domain which is at least 90%, 91%, 92%, 93%, 94%, 95% 96%, 97%, 98% or 99% similar or identical to the sequence given in SEQ ID NO:29 and a light chain variable domain which is at least 90%, 91%, 92%, 93%, 94%, 95% 96%, 97%, 98% or 99% similar or identical to the sequence given in SEQ ID NO:15 but wherein the antibody molecule has the sequence given in SEQ ID NO: 1 for CDR-H1, the sequence given in SEQ ID NO: 2 for CDR-H2, the sequence given in SEQ ID NO: 3 for CDR-H3, the sequence given in SEQ ID NO: 4 for CDR-L1, the sequence given in SEQ ID NO: 5 for CDR-L2 and the sequence given in SEQ ID NO:6 for CDR-L3.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains). Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656,).

The antibody molecules of the present invention may comprise a complete antibody molecule having full length heavy and light chains or a fragment thereof and may be, but are not limited to Fab, modified Fab, Fab′, modified Fab′, F(ab′)2, Fv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in International patent applications WO2005/003169, WO2005/003170 and WO2005/003171. Multi-valent antibodies may comprise multiple specificities e.g bispecific or may be monospecific (see for example WO 92/22853, WO05/113605, WO2009/040562 and WO2010/035012).

In one embodiment the antibody molecule of the present disclosure is an antibody Fab′ fragment comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:36.

In one embodiment the antibody molecule of the present disclosure is a full length IgG1 antibody comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:72.

In one embodiment the antibody molecule of the present disclosure is a full length IgG4 format comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:87.

In one embodiment the antibody molecule of the present disclosure is a full length IgG4P format comprising the variable regions shown in SEQ ID NOs: 15 and 29 for example for the light and heavy chain respectively. In one embodiment the antibody molecule has a light chain comprising the sequence given in SEQ ID NO:22 and a heavy chain comprising the sequence given in SEQ ID NO:43.

IgG4P as employed herein is a mutation of the wild-type IgG4 isotype where amino acid 241 is replaced by proline see for example where serine at position 241 has been changed to proline as described in Angal et al., Molecular Immunology, 1993, 30 (1), 105-108. In one embodiment the antibody according to the present disclosure is provided as an FcRn binding antibody fusion protein which comprises an immunoglobulin moiety, for example a Fab or Fab′ fragment, and one or two single domain antibodies (dAb) linked directly or indirectly thereto, for example as described in WO2009/040562, WO2010035012, WO2011/030107, WO2011/061492 and WO2011/086091 all incorporated herein by reference.

In one embodiment the fusion protein comprises two domain antibodies, for example as a variable heavy (VH) and variable light (VL) pairing, optionally linked by a disulphide bond.

In one embodiment the Fab or Fab′ element of the fusion protein has the same or similar specificity to the single domain antibody or antibodies. In one embodiment the Fab or Fab′ has a different specificity to the single domain antibody or antibodies, that is to say the fusion protein is multivalent. In one embodiment a multivalent fusion protein according to the present invention has an albumin binding site, for example a VH/VL pair therein provides an albumin binding site. In one such embodiment the heavy chain comprises the sequence given in SEQ ID NO:50 and the light chain comprises the sequence given in SEQ ID NO:46 or SEQ ID NO:78.

In one embodiment the Fab or Fab′ according to the present disclosure is conjugated to a PEG molecule or human serum albumin.

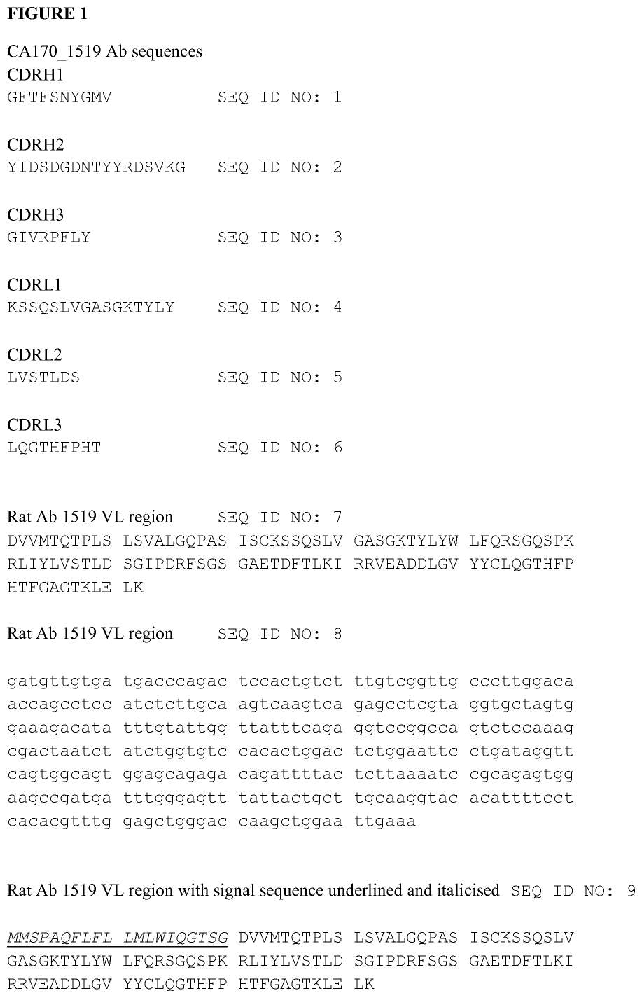

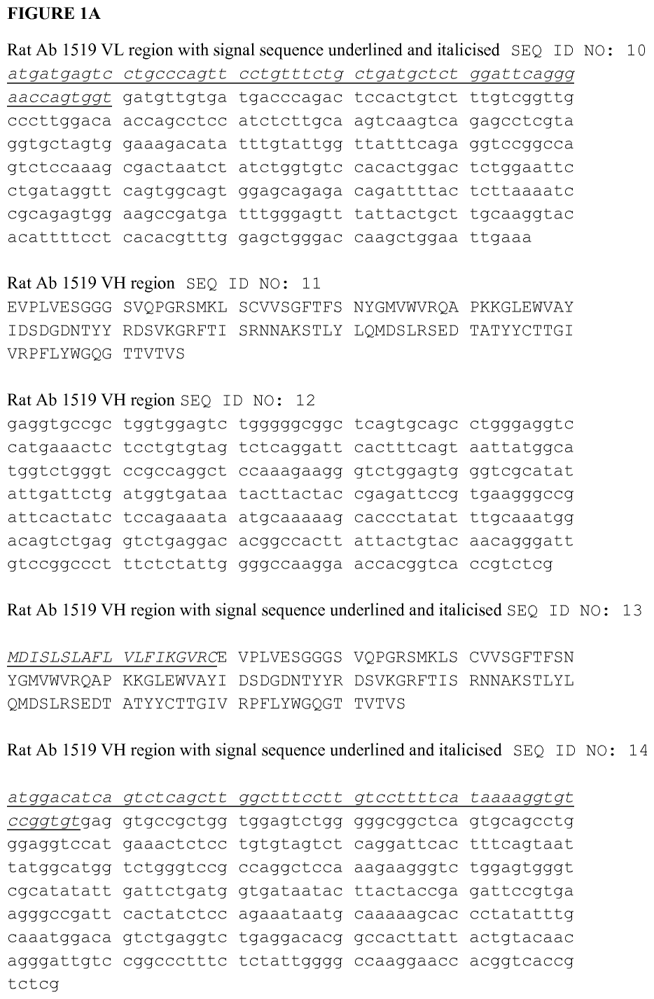

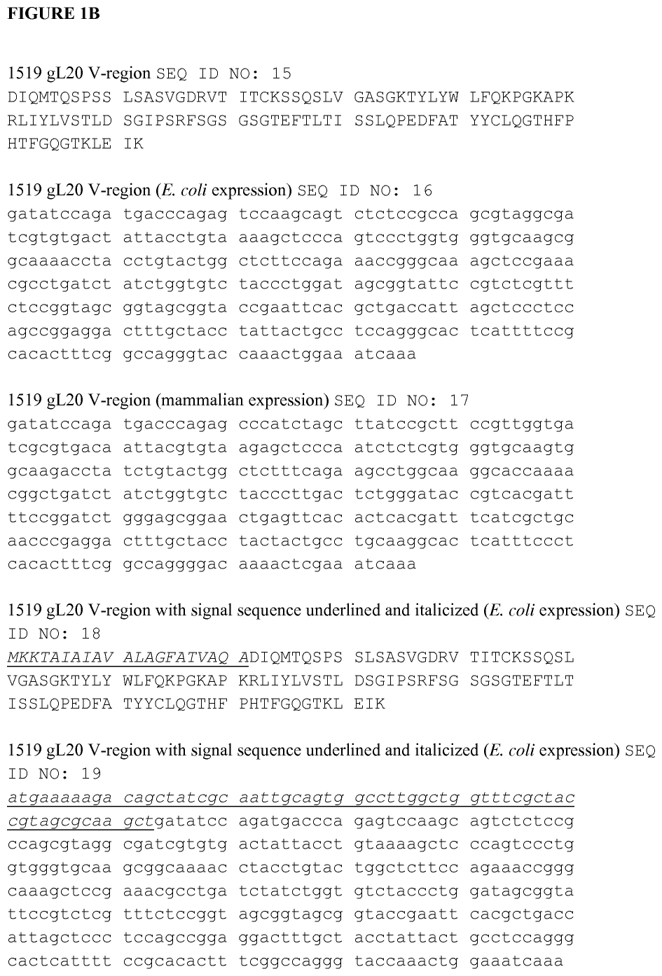

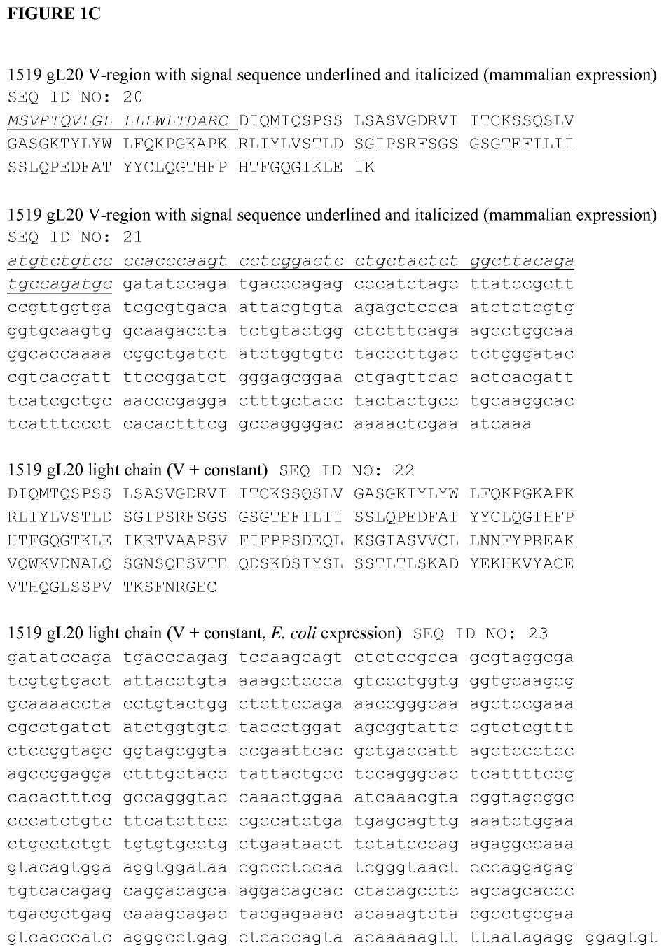

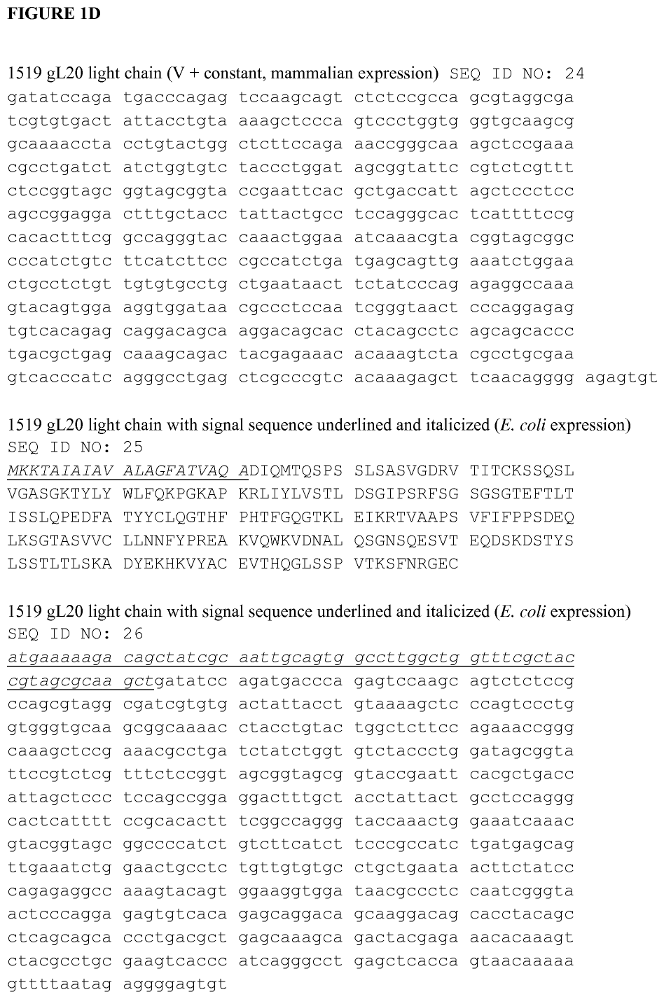

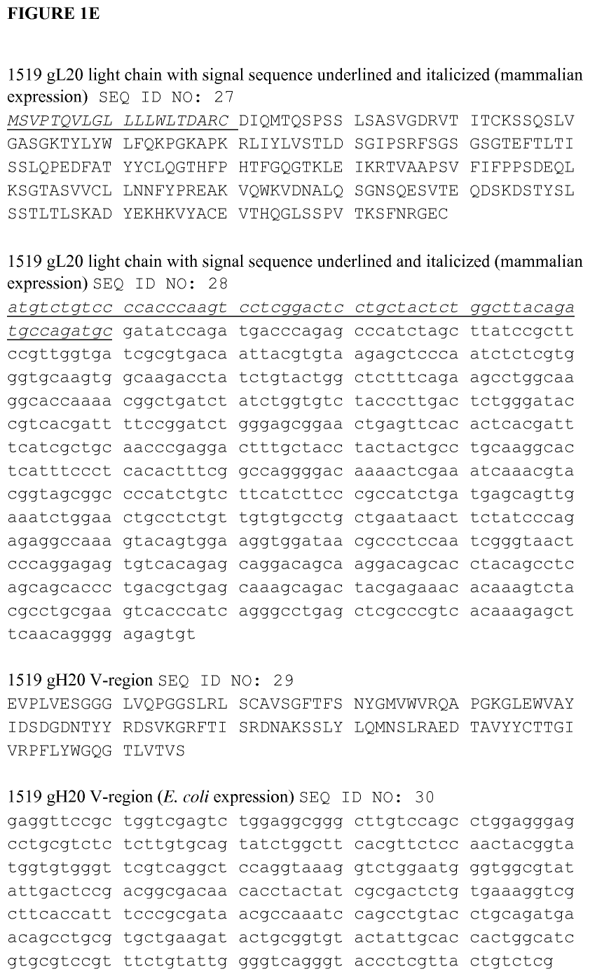

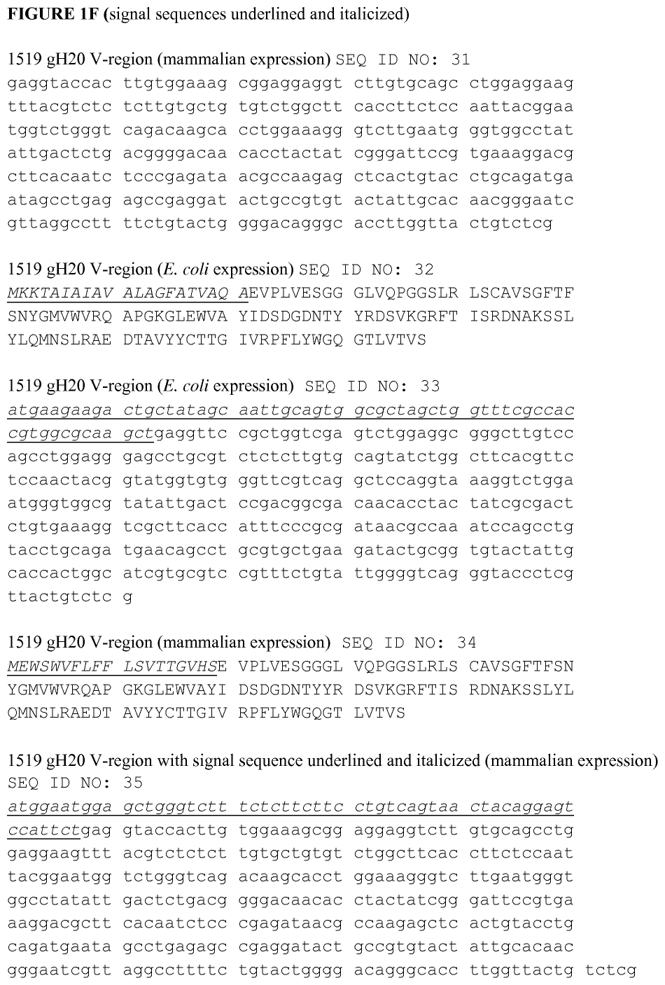

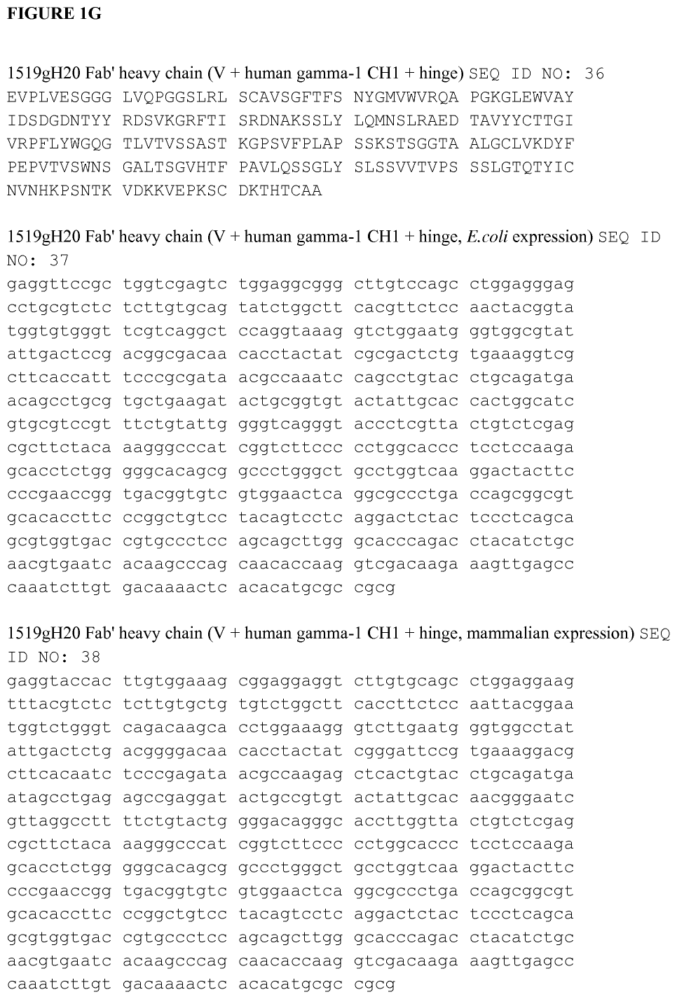

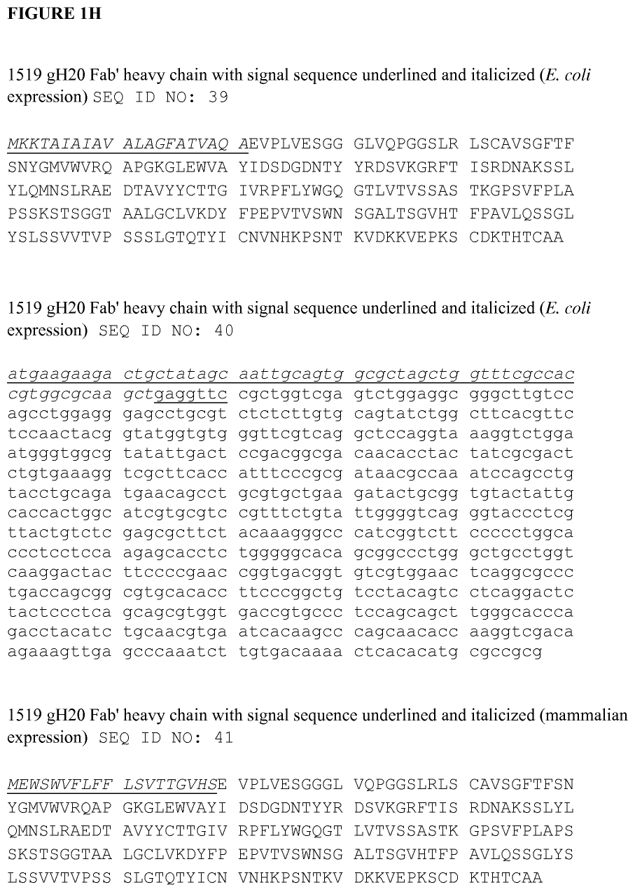

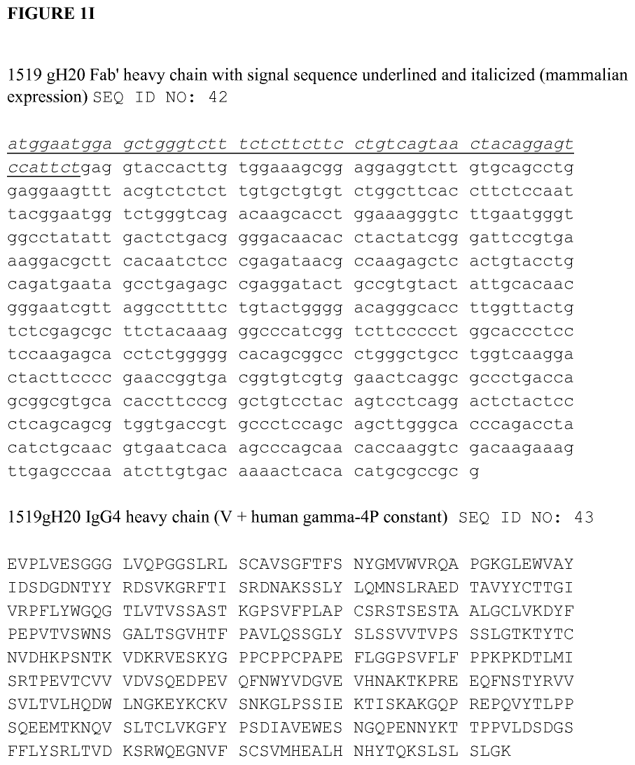

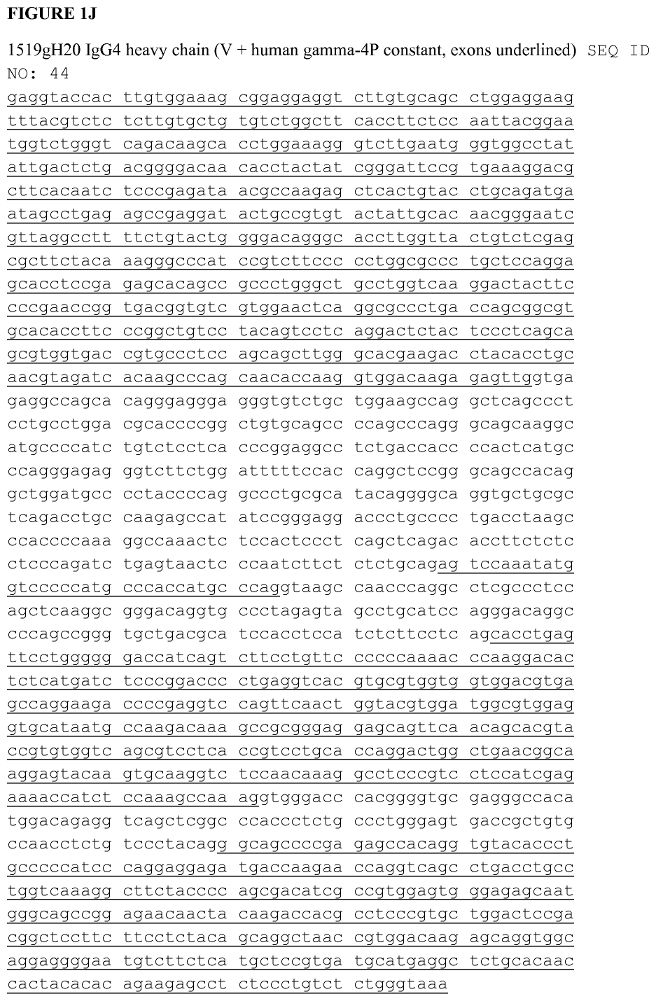

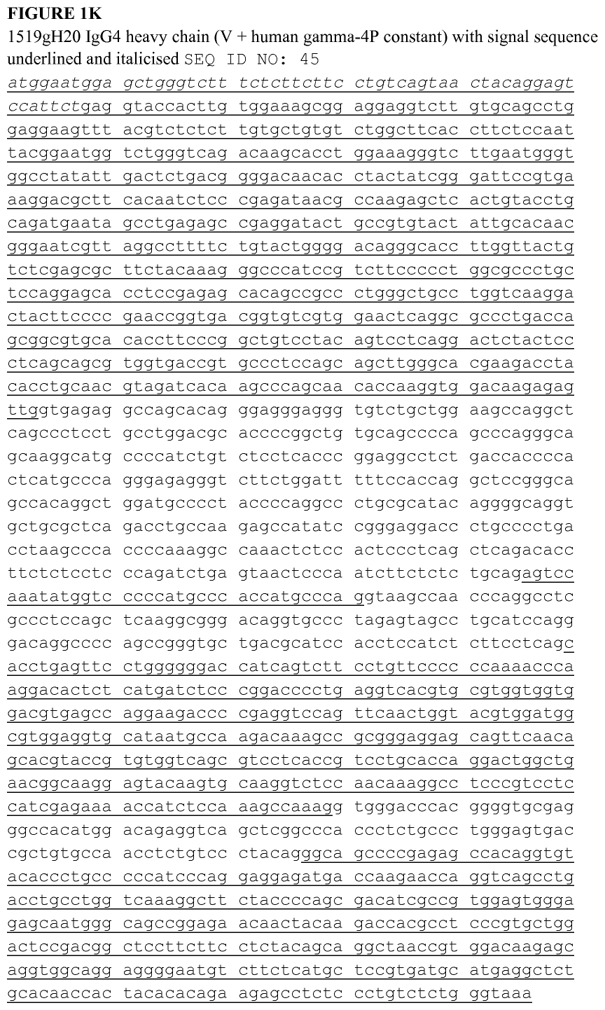

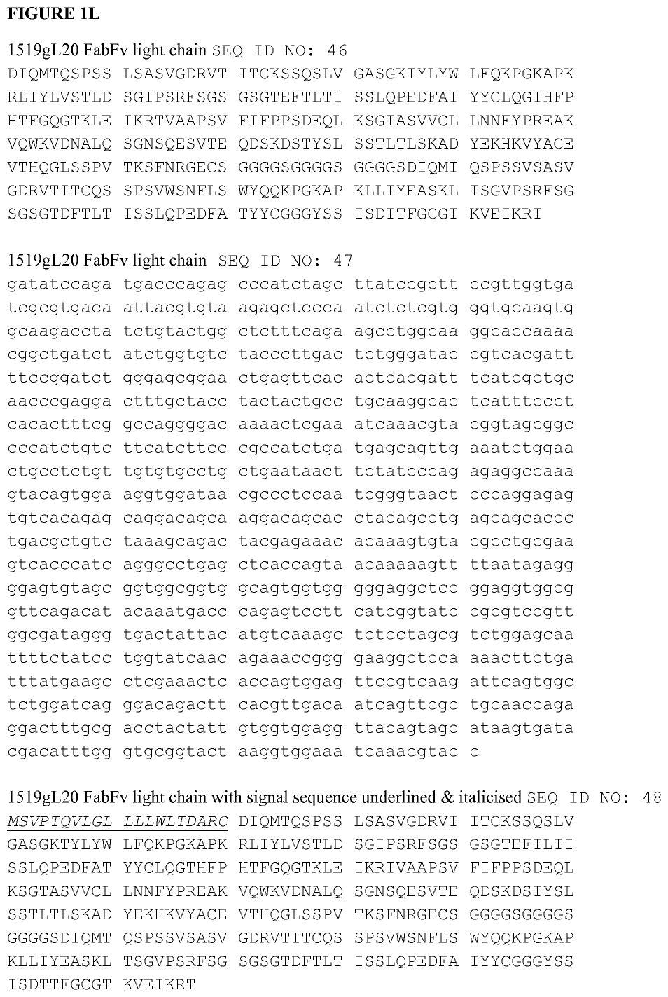

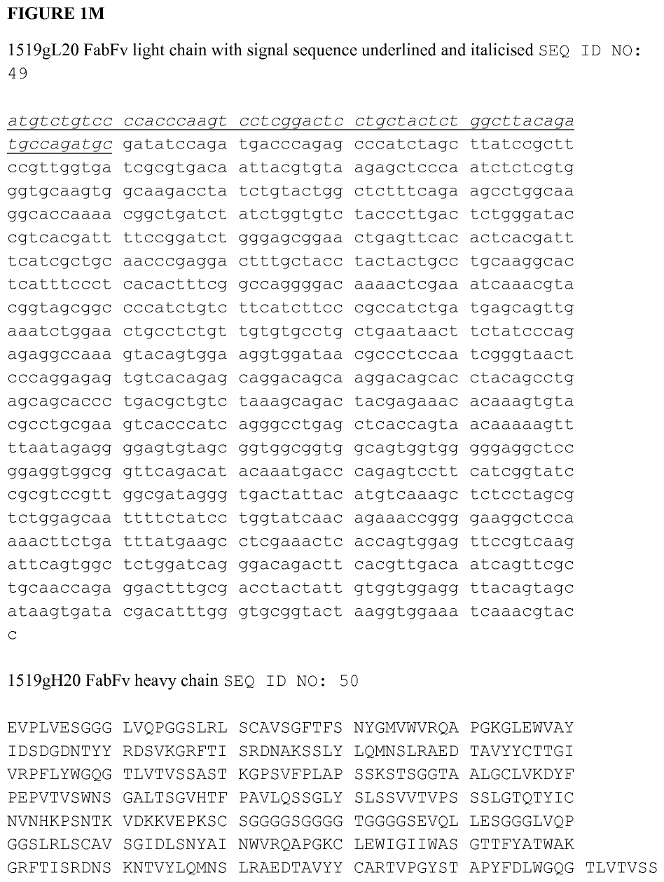

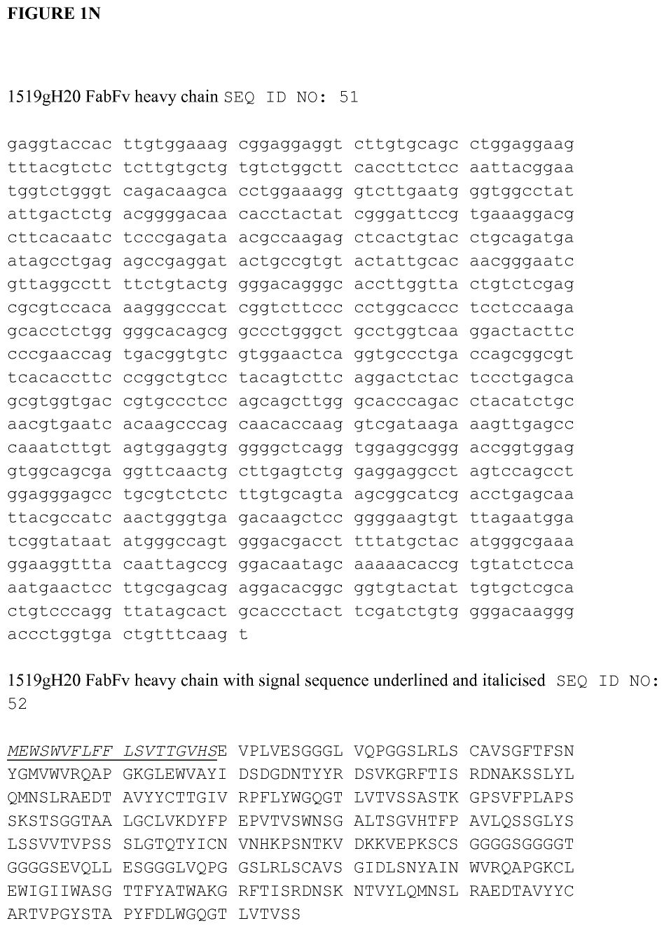

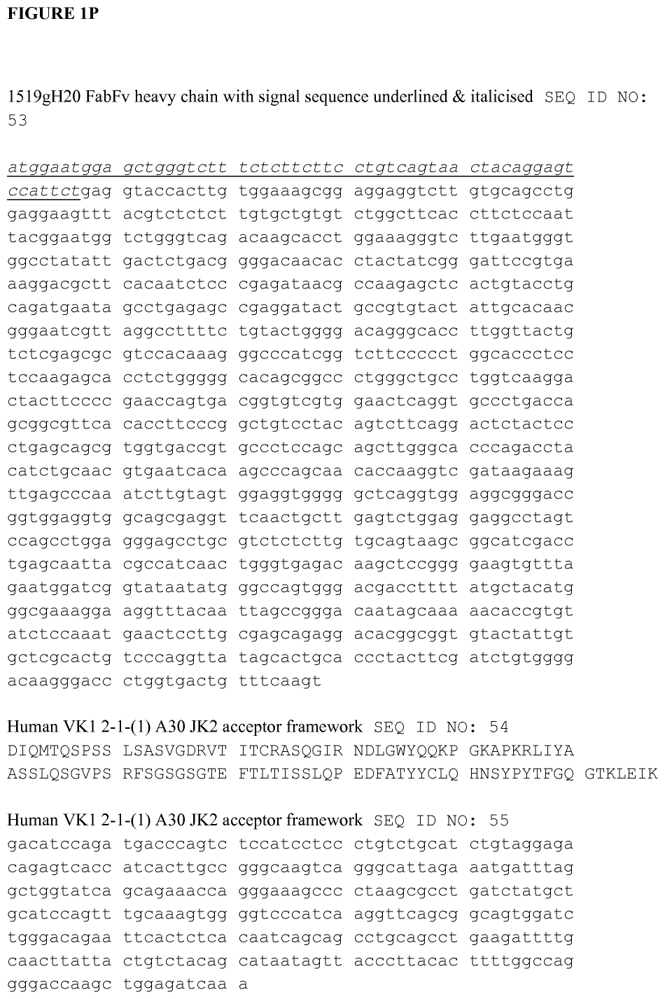

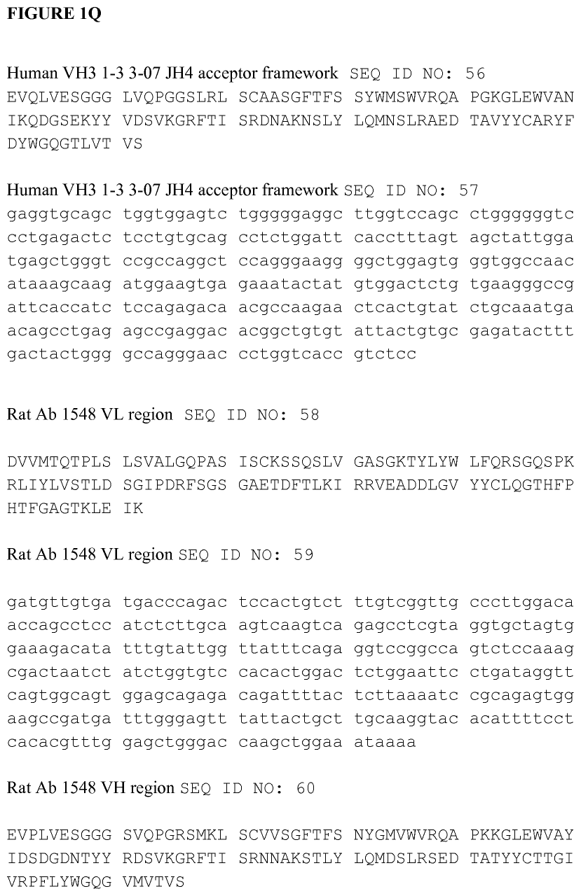

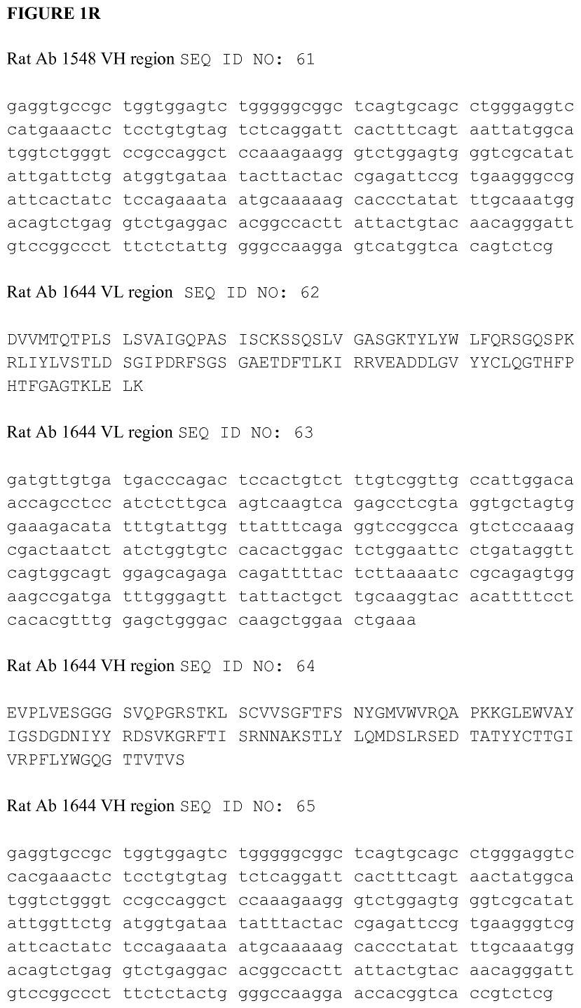

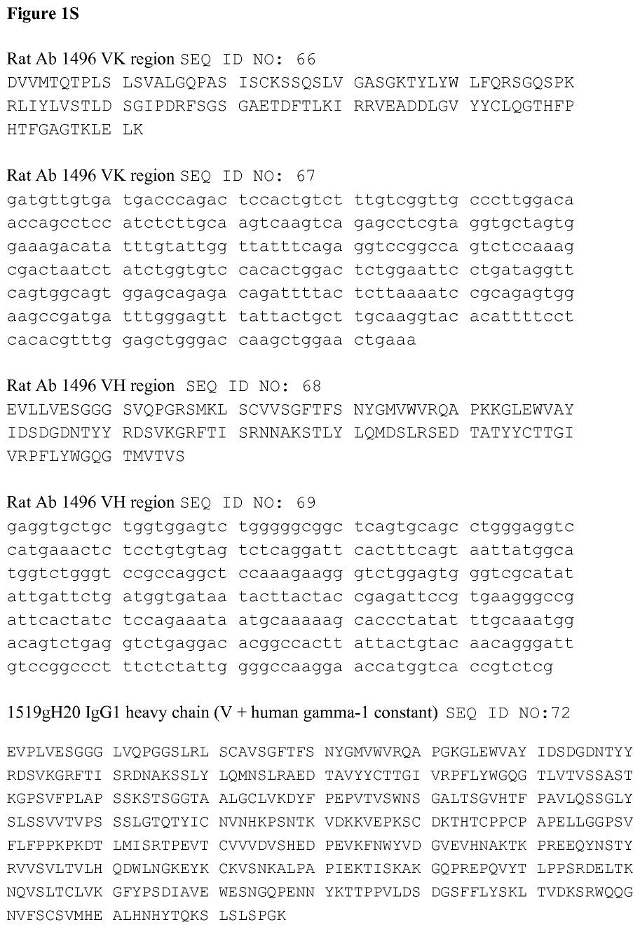

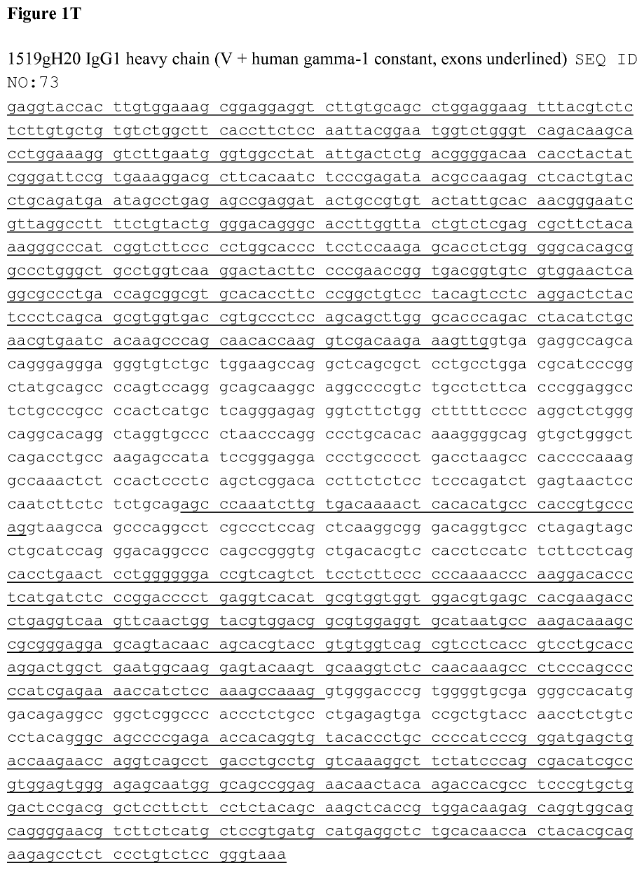

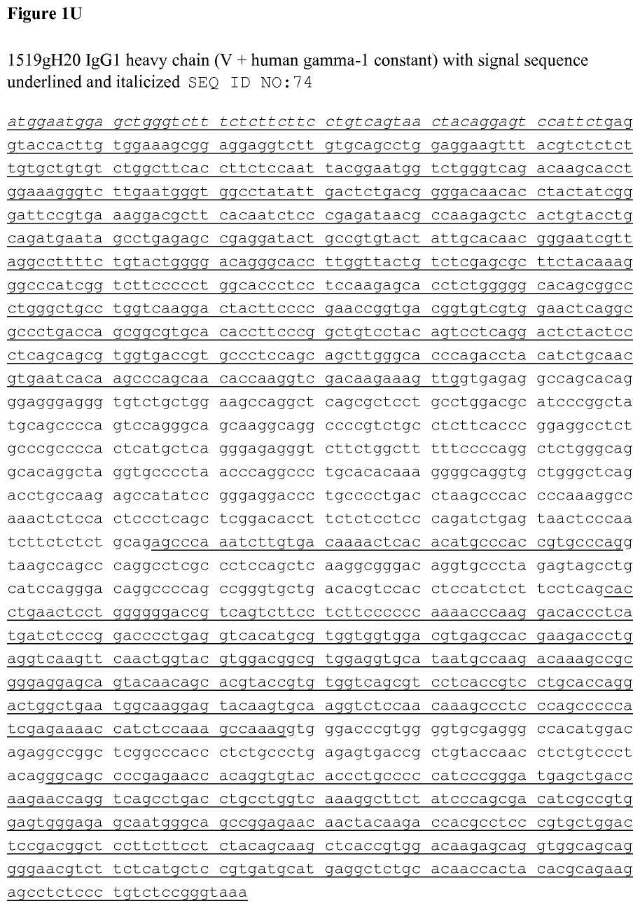

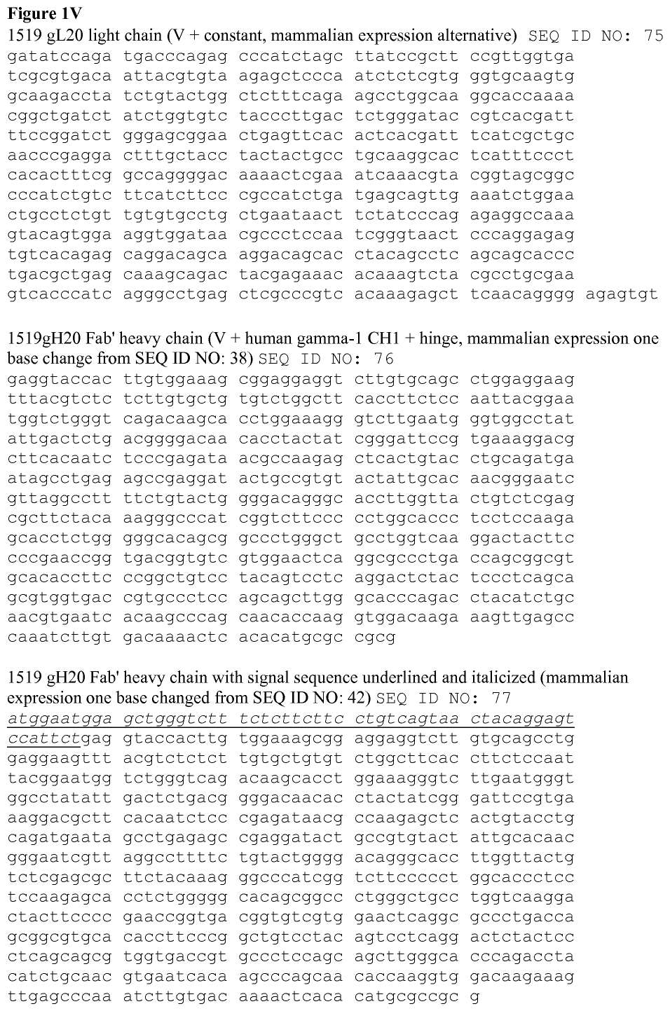

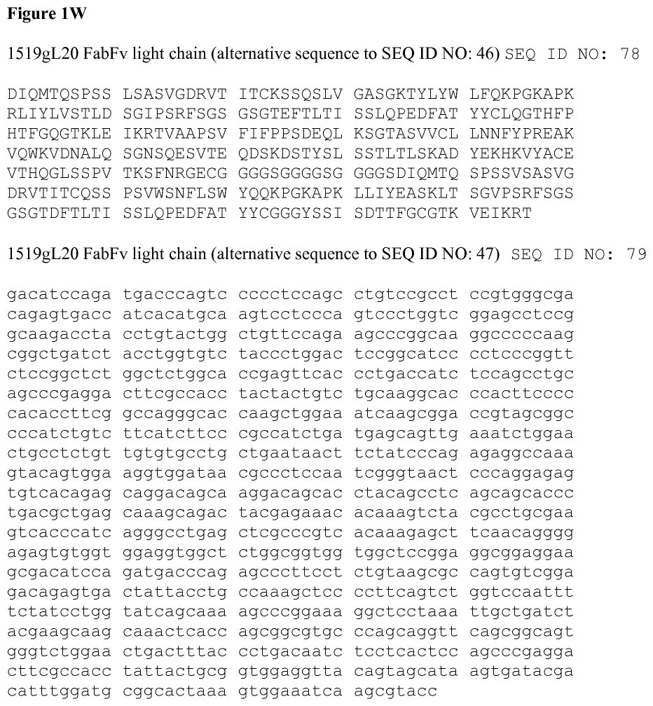

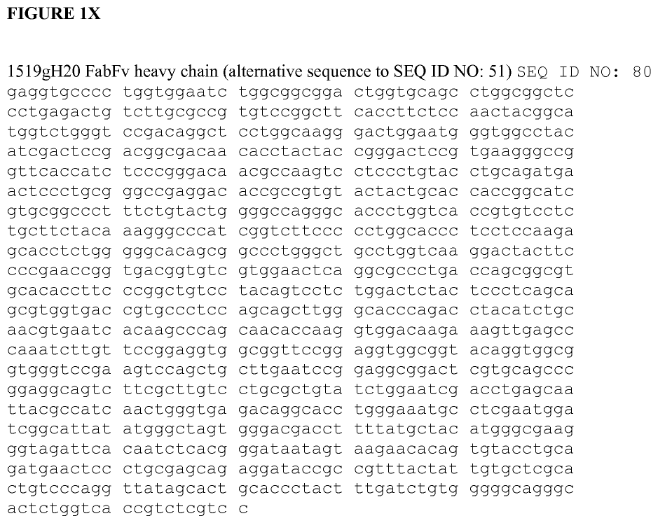

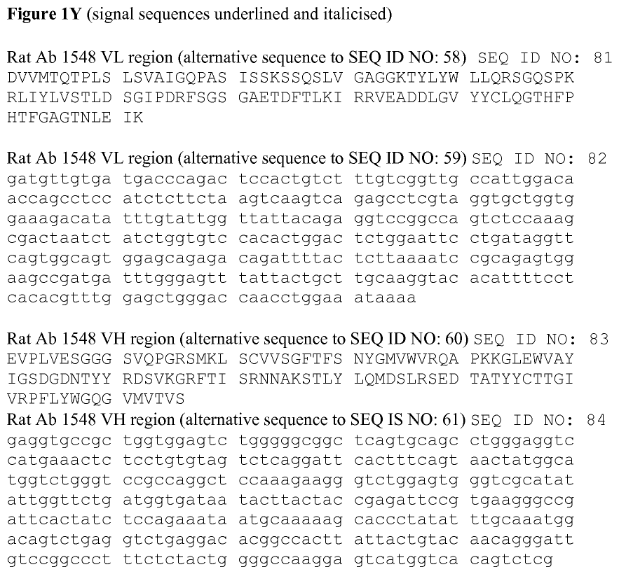

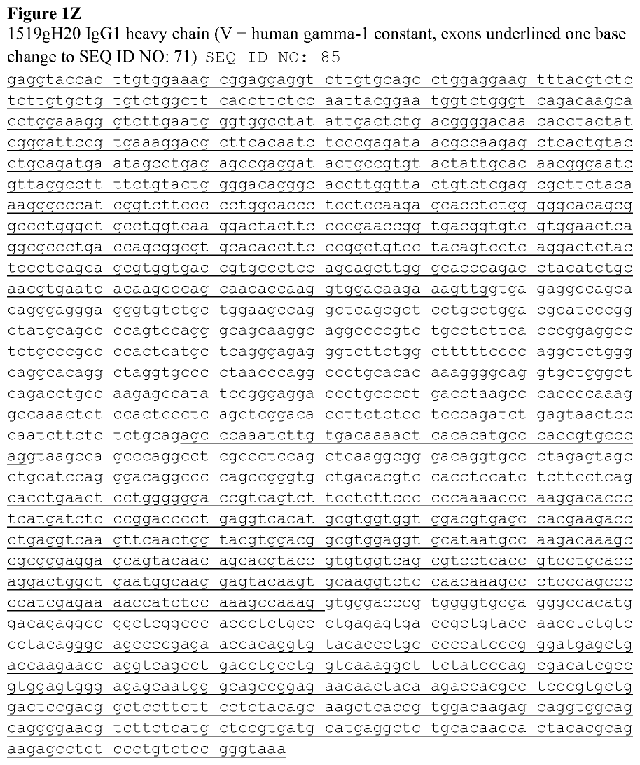

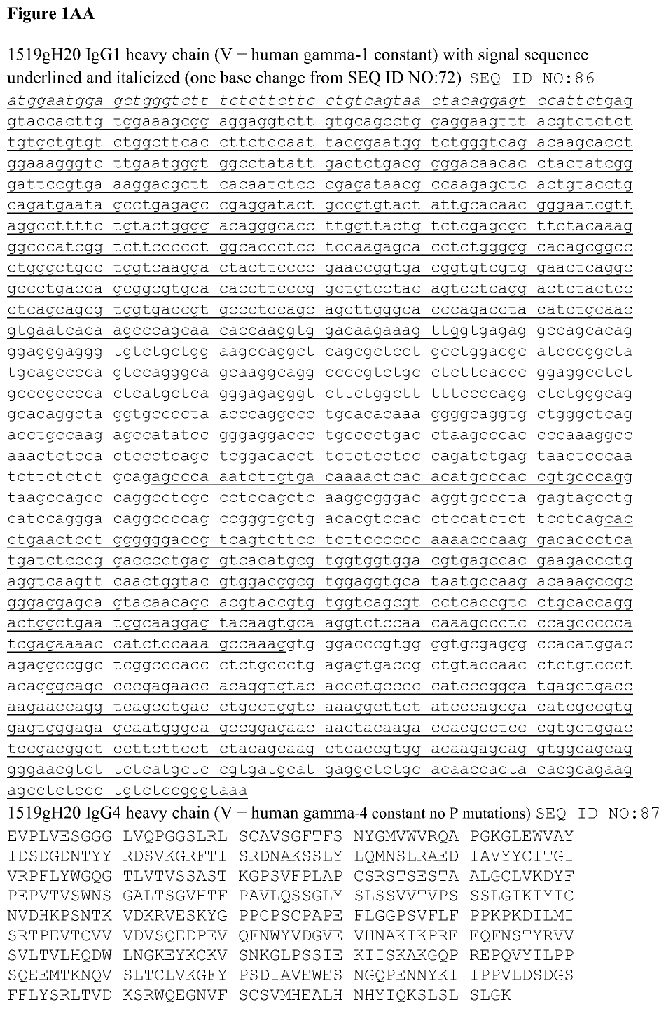

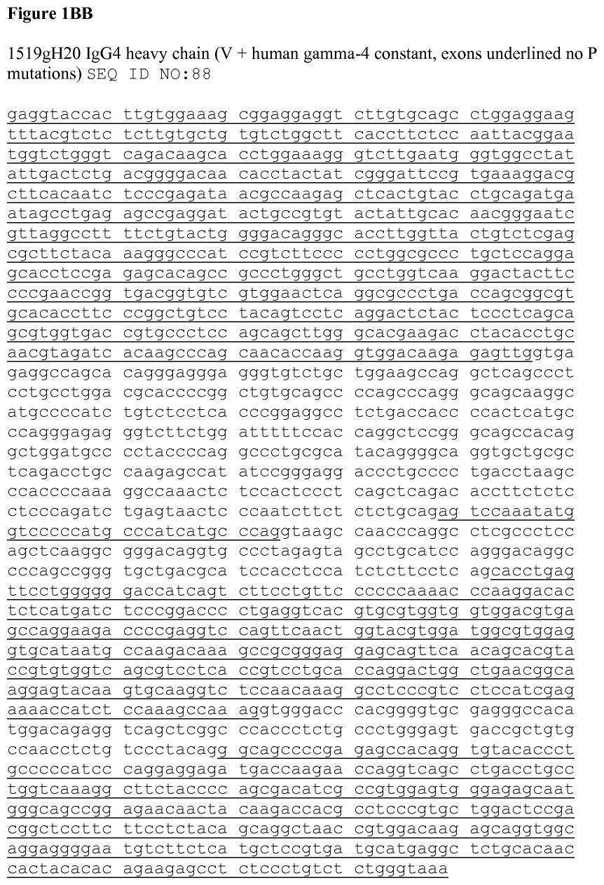

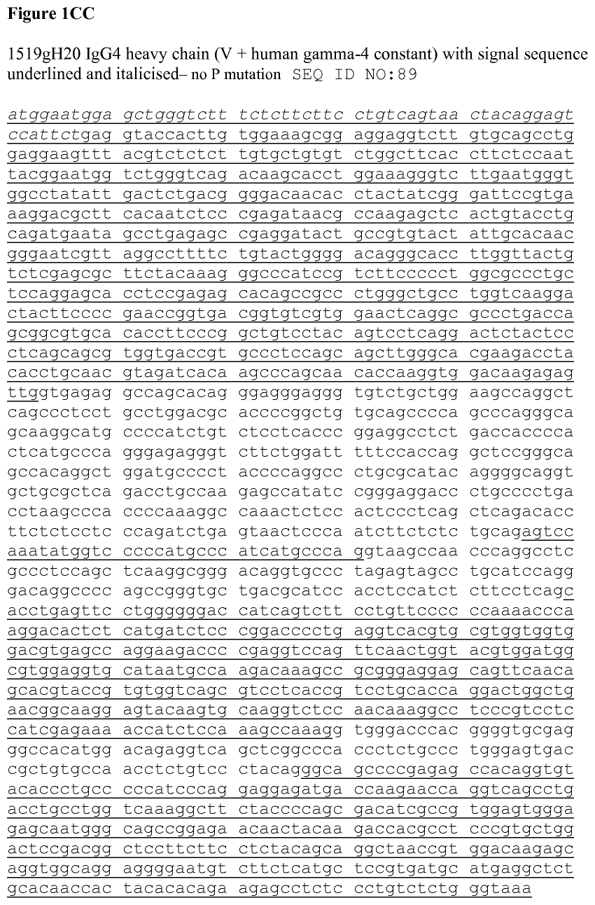

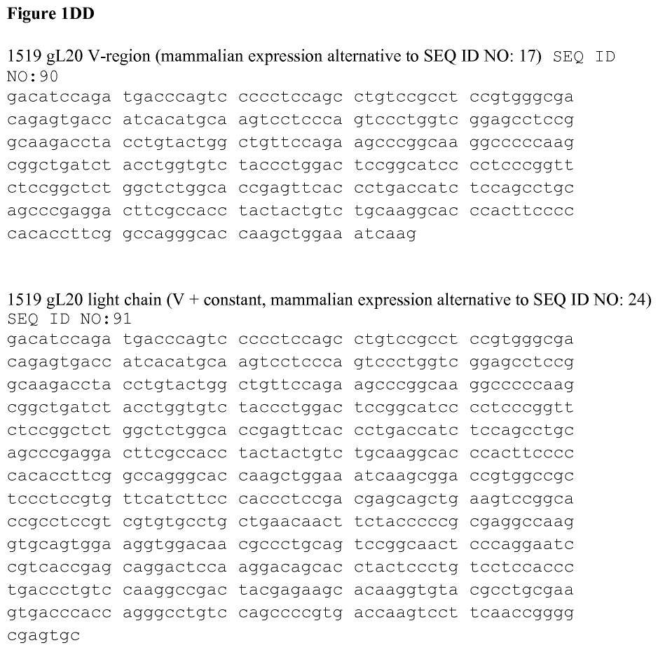

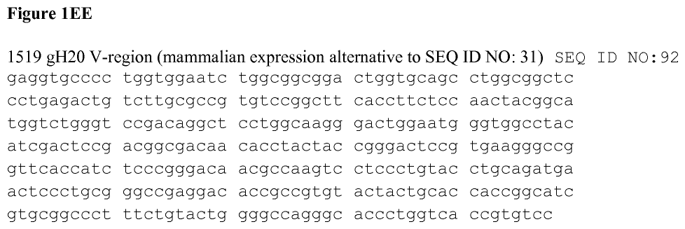

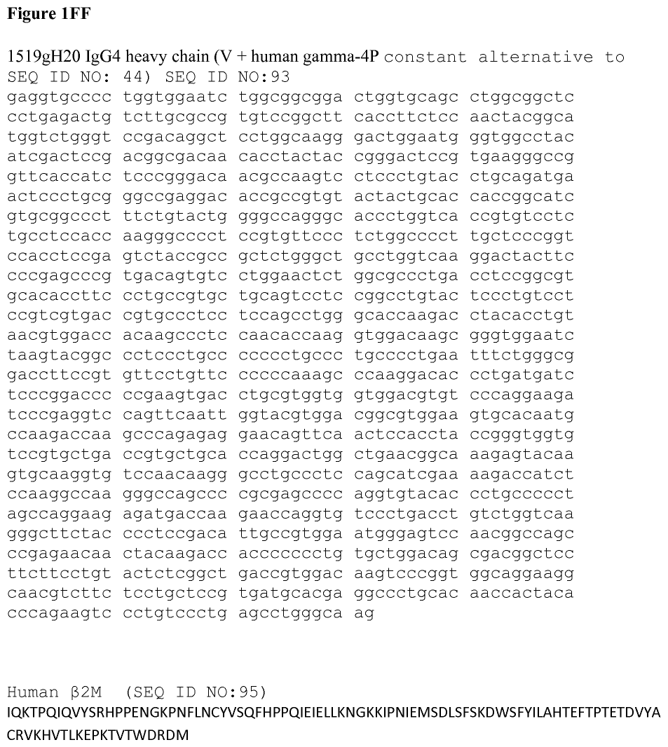

CA170_01519g57 and 1519 and 1519.g57 are employed inchangeably herein and are used to refer to a specific pair of antibody variable regions which may be used in a number of different formats. These variable regions are the heavy chain sequence given in SEQ ID NO:29 and the light chain sequence given in SEQ ID NO:15 (FIG. 1).

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example IgG4 molecules in which the serine at position 241 has been changed to proline as described in Angal et al., Molecular Immunology, 1993, 30 (1), 105-108 may be used. It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995). Accordingly, the C-terminal lysine of the antibody heavy chain may be absent.

In one embodiment the antibody heavy chain comprises a CH1 domain and the antibody light chain comprises a CL domain, either kappa or lambda.

In one embodiment the light chain has the sequence given in SEQ ID NO:22 and the heavy chain has the sequence given in SEQ ID NO:43.

In one embodiment the light chain has the sequence given in SEQ ID NO:22 and the heavy chain has the sequence given in SEQ ID NO:72.

In one embodiment a C-terminal amino acid from the antibody molecule is cleaved during post-translation modifications.

In one embodiment an N-terminal amino acid from the antibody molecule is cleaved during post-translation modifications.

Also provided by the present disclosure is a specific region or epitope of human FcRn which is bound by an antibody provided by the present invention, in particular an antibody comprising the heavy chain sequence gH20 (SEQ ID NO:29) and/or the light chain sequence gL20 (SEQ ID NO:15).

This specific region or epitope of the human FcRn polypeptide can be identified by any suitable epitope mapping method known in the art in combination with any one of the antibodies provided by the present invention. Examples of such methods include screening peptides of varying lengths derived from FcRn for binding to the antibody of the present invention with the smallest fragment that can specifically bind to the antibody containing the sequence of the epitope recognised by the antibody. The FcRn peptides may be produced synthetically or by proteolytic digestion of the FcRn polypeptide. Peptides that bind the antibody can be identified by, for example, mass spectrometric analysis. In another example, NMR spectroscopy or X-ray crystallography can be used to identify the epitope bound by an antibody of the present invention. Once identified, the epitopic fragment which binds an antibody of the present invention can be used, if required, as an immunogen to obtain additional antibodies which bind the same epitope.

In one embodiment the antibody of the present disclosure binds the human FcRn alpha chain extracellular sequence as shown below:

(SEQ ID NO: 94)
AESHLSLLYH LTAVSSPAPG TPAFWVSGWL GPQQYLSYNS
LRGEAEPCGA WVWENQVSWY WEKETTDLRI KEKLFLEAFK
ALGGKGPYTL QGLLGCELGP DNTSVPTAKF ALNGEEFMNF
DLKQGTWGGD WPEALAISQR WQQQDKAANK ELTFLLFSCP
HRLREHLERG RGNLEWKEPP SMRLKARPSS PGFSVLTCSA
FSFYPPELQL RFLRNGLAAG TGQGDFGPNS DGSFHASSSL
TVKSGDEHHY CCIVQHAGLA QPLRVELESPAKSS.

The residues underlined are those known to be critical for the interaction of human FcRn with the Fc region of human IgG and those residues highlighted in bold are those involved in the interaction of FcRn with the 1519 antibody of the present disclosure comprising the heavy chain sequence gH20 (SEQ ID NO:29) and the light chain sequence gL20 (SEQ ID NO:15).

In one example, the present invention provides an anti-FcRn antibody molecule which binds an epitope of human FcRn which comprises at least one amino acid selected from the group consisting of residues V105, P106, T107, A108 and K109 of SEQ ID NO:94 and at least one residue, for example at least 2, 3, 4, 5, 6, 7, 8, 9 or 10 residues selected from the group consisting of P100, E115, E116, F117, M118, N119, F120, D121, L122, K123, Q124, G128, G129, D130, W131, P132 and E133 of SEQ ID NO:94.

In one example the epitope of the antibody molecule is determined by X-ray crystallography using the FcRn alpha chain extracellular sequence (SEQ ID NO:94) in complex with 02M.

In one example, the present invention provides an anti-FcRn antibody molecule which binds an epitope of human FcRn which comprises at least one amino acid selected from the group consisting of residues V105, P106, T107, A108 and K109 of SEQ ID NO:94 and at least one residue, for example at least 2, 3, 4, 5, 6, 7, 8, 9 or 10 residues, selected from the group consisting of E115, E116, F117, M118, N119, F120, D121, L122, K123 and Q124 of SEQ ID NO:94.

In one example, the present invention provides an anti-FcRn antibody molecule which binds an epitope of human FcRn which comprises at least two, three, four or five amino acids selected from the group consisting of residues V105, P106, T107, A108 and K109 of SEQ ID NO:94 and at least one residue selected from the group consisting of E115, E116, F117, M118, N119, F120, D121, L122, K123 and Q124 of SEQ ID NO:94.

In one example, the present invention provides an anti-FcRn antibody molecule which binds an epitope of human FcRn which comprises at least one amino acid selected from the group consisting of residues V105, P106, T107, A108 and K109 of SEQ ID NO:94 and at least one residue selected from the group consisting of P100, E115, E116, F117, M118, N119, F120, D121, L122, K123, Q124, G128, G129, D130, W131, P132 and E133 of SEQ ID NO:94.

In one example, the present invention provides an anti-FcRn antibody molecule which binds an epitope of human FcRn which comprises at least one amino acid selected from the group consisting of residues V105, P106, T107, A108 and K109 of SEQ ID NO:94 and at least one residue selected from the group consisting of P100, M118, N119, F120, D121, L122, K123, Q124 and G128 of SEQ ID NO:94.

In one example, the present invention provides an anti-FcRn antibody molecule which binds an epitope of human FcRn which comprises residues V105, P106, T107, A108 and K109 of SEQ ID NO:94 and at least one residue selected from the group consisting of P100, M118, N119, F120, D121, L122, K123, Q124 and G128 of SEQ ID NO:94.

In one example, the present invention provides an anti-FcRn antibody molecule which binds an epitope of human FcRn which comprises residues V105, P106, T107, A108 and K109 of SEQ ID NO:94 and at least one residue selected from the group consisting of P100, E115, E116, F117, M118, N119, F120, D121, L122, K123, Q124, G128, G129, D130, W131, P132 and E133 of SEQ ID NO:94.

In one example, the present invention provides an anti-FcRn antibody molecule which binds an epitope of human FcRn which comprises residues P100, V105, P106, T107, A108 and K109 of SEQ ID NO:94 and at least one residue selected from the group consisting of E115, E116, F117, M118, N119, F120, D121, L122, K123, Q124, G128, G129, D130, W131, P132 and E133 of SEQ ID NO:94.

In one example ‘at least one residue’ may be 1, 2, 3, 4, 5, 6, 7, 8, 9, 10, 11, 12, 13, 14, 15 or 16 residues.

In one example the present invention provides an anti-FcRn antibody molecule which binds an epitope of human FcRn which comprises or consists of residues 100, 105 to 109, 115 to 124 and 129 to 133 of SEQ ID NO: 94.

Antibodies which cross-block the binding of an antibody molecule according to the present invention in particular, an antibody molecule comprising the heavy chain sequence given in SEQ ID NO:29 and the light chain sequence given in SEQ ID NO:15 may be similarly useful in blocking FcRn activity. Accordingly, the present invention also provides an anti-FcRn antibody molecule, which cross-blocks the binding of any one of the antibody molecules described herein above to human FcRn and/or is cross-blocked from binding human FcRn by any one of those antibodies. In one embodiment, such an antibody binds to the same epitope as an antibody described herein above. In another embodiment the cross-blocking neutralising antibody binds to an epitope which borders and/or overlaps with the epitope bound by an antibody described herein above.

Cross-blocking antibodies can be identified using any suitable method in the art, for example by using competition ELISA or BIAcore assays where binding of the cross blocking antibody to human FcRn prevents the binding of an antibody of the present invention or vice versa. Such cross blocking assays may use isolated natural or recombinant FcRn or a suitable fusion protein/polypeptide. In one example binding and cross-blocking is measured using recombinant human FcRn extracellular domain (SEQ ID NO:94). In one example the recombinant human FcRn alpha chain extracellular domain is used in a complex with β2 microglobulin (P2M) (SEQ ID NO:95).

In one embodiment there is provided an anti-FcRn antibody molecule which blocks FcRn binding to IgG and which cross-blocks the binding of an antibody whose heavy chain comprises the sequence given in SEQ ID NO:29 and whose light chain comprises the sequence given in SEQ ID NO:15 to human FcRn. In one embodiment the cross-blocking antibodies provided by the present invention inhibit the binding of an antibody comprising the heavy chain sequence given in SEQ ID NO:29 and the light chain sequence given in SEQ ID NO:15 by greater than 80%, for example by greater than 85%, such as by greater than 90%, in particular by greater than 95%.

Alternatively or in addition, anti-FcRn antibodies according to this aspect of the invention may be cross-blocked from binding to human FcRn by an antibody comprising the heavy chain sequence given in SEQ ID NO:29 and the light chain sequence given in SEQ ID NO:15. Also provided therefore is an anti-FcRn antibody molecule which blocks FcRn binding to IgG and which is cross-blocked from binding human FcRn by an antibody comprising the heavy chain sequence given in SEQ ID NO:29 and the light chain sequence given in SEQ ID NO:15. In one embodiment the anti-FcRn antibodies provided by this aspect of the invention are inhibited from binding human FcRn by an antibody comprising the heavy chain sequence given in SEQ ID NO:29 and the light chain sequence given in SEQ ID NO:15 by greater than 80%, for example by greater than 85%, such as by greater than 90%, in particular by greater than 95%.

In one embodiment the cross-blocking antibodies provided by the present invention are fully human. In one embodiment the cross-blocking antibodies provided by the present invention are humanised. In one embodiment the cross-blocking antibodies provided by the present invention have an affinity for human FcRn of 100 pM or less. In one embodiment the cross-blocking antibodies provided by the present invention have an affinity for human FcRn of 50 pM or less. Affinity can be measured using the methods described herein below.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or solvent accessible surface thereof carries no net electrical charge. In one example, the FcRn antibody and fragments of the invention may be engineered to have an appropriate isoelectric point. This may lead to antibodies and/or fragments with more robust properties, in particular suitable solubility and/or stability profiles and/or improved purification characteristics.

Thus in one aspect the invention provides a humanised FcRn antibody engineered to have an isoelectric point different to that of the originally identified antibody. The antibody may, for example be engineered by replacing an amino acid residue such as replacing an acidic amino acid residue with one or more basic amino acid residues. Alternatively, basic amino acid residues may be introduced or acidic amino acid residues can be removed. Alternatively, if the molecule has an unacceptably high pI value acidic residues may be introduced to lower the pI, as required. It is important that when manipulating the pI care must be taken to retain the desirable activity of the antibody or fragment. Thus in one embodiment the engineered antibody or fragment has the same or substantially the same activity as the “unmodified” antibody or fragment.

Programs such as ** ExPASY http://www.expasy.ch/tools/pi_tool.html, and

http://www.iut-arles.up.univ-mrs.fr/w3bb/d_abim/compo-p.html, may be used to predict the isoelectric point of the antibody or fragment.

The antibody molecules of the present invention suitably have a high binding affinity, in particular in the nanomolar range. Affinity may be measured using any suitable method known in the art, including BIAcore, as described in the Examples herein, using isolated natural or recombinant FcRn or a suitable fusion protein/polypeptide. In one example affinity is measured using recombinant human FcRn extracellular domain as described in the Examples herein (SEQ ID NO:94) and in WO2014/019727. In one example affinity is measured using the recombinant human FcRn alpha chain extracellular domain (SEQ ID NO:94) in association with β2 microglobulin (β2M) (SEQ ID NO:95). Suitably the antibody molecules of the present invention have a binding affinity for isolated human FcRn of about 1 nM or lower. In one embodiment the antibody molecule of the present invention has a binding affinity of about 500 pM or lower (i.e. higher affinity). In one embodiment the antibody molecule of the present invention has a binding affinity of about 250 pM or lower. In one embodiment the antibody molecule of the present invention has a binding affinity of about 200 pM or lower. In one embodiment the present invention provides an anti-FcRn antibody with a binding affinity of about 100 pM or lower. In one embodiment the present invention provides a humanised anti-FcRn antibody with a binding affinity of about 100 pM or lower. In one embodiment the present invention provides an anti-FcRn antibody with a binding affinity of 50 pM or lower.

Importantly the antibodies of the present invention are able to bind human FcRn at both pH6 and pH7.4 with comparable binding affinity. Advantageously therefore the antibodies are able to continue to bind FcRn even within the endosome, thereby maximising the blocking of FcRn binding to IgG.

In one embodiment the present invention provides an anti-FcRn antibody with a binding affinity of 100 pM or lower when measured at pH6 and pH7.4. In one embodiment the antibody for use in the invention is an anti-FcRn antibody with a binding affinity of 50 pM or lower when measured at pH6 and pH7.4.

The affinity of an antibody or binding fragment of the present invention, as well as the extent to which a binding agent (such as an antibody) inhibits binding, can be determined by one of ordinary skill in the art using conventional techniques, for example those described by Scatchard et al. (Ann. KY. Acad. Sci. 51:660-672 (1949)) or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

In the present invention affinity of the test antibody molecule is typically determined using SPR as follows. The test antibody molecule is captured on the solid phase and human FcRn alpha chain extracellular domain in non-covalent complex with 02M is run over the captured antibody in the mobile phase and affinity of the test antibody molecule for human FcRn determined. The test antibody molecule may be captured on the solid phase chip surface using any appropriate method, for example using an anti-Fc or anti Fab′ specific capture agent. In one example the affinity is determined at pH6. In one example the affinity is determined at pH7.4.

It will be appreciated that the affinity of antibodies provided by the present invention may be altered using any suitable method known in the art. The present invention therefore also relates to variants of the antibody molecules of the present invention, which have an improved affinity for FcRn. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation.

In one embodiment the antibody molecules of the present invention block human FcRn activity. Assays suitable for determining the ability of an antibody to block FcRn are described in WO2014/019727. Suitable assays for determining whether antibodies block FcRn interaction with circulating IgG molecules are also described in WO2014/019727 along with a suitable assay for determining the ability of an antibody molecule to block IgG recycling in vitro.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

The term effector molecule as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rheniums¹⁸⁸; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

In one embodiment a half-life provided by an effector molecule which is independent of FcRn is advantageous.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment the polymer is albumin or a fragment thereof, such as human serum albumin or a fragment thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an ca-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment, Fab′ fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL40K (obtainable from Nektar, formerly Shearwater).

Alternative sources of PEG linkers include NOF who supply GL2-400MA3 (wherein m in the structure below is 5) and GL2-400MA (where m is 2) and n is approximately 450:

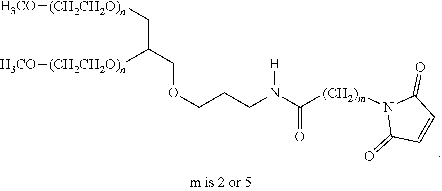

That is to say each PEG is about 20,000 Da.

Thus in one embodiment the PEG is 2,3-Bis(methylpolyoxyethylene-oxy)-1-{[3-(6-maleimido-1-oxohexyl)amino]propyloxy} hexane (the 2 arm branched PEG, —CH₂)₃NHCO(CH₂)₅-MAL, Mw 40,000 known as SUNBRIGHT GL2-400MA3.

Further alternative PEG effector molecules of the following type:

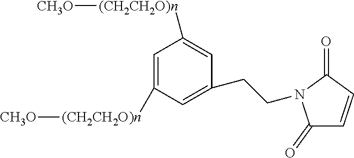

are available from Dr Reddy, NOF and Jenkem.

In one embodiment there is provided an antibody which is PEGylated (for example with a PEG described herein), attached through a cysteine amino acid residue at or about amino acid 226 in the chain, for example amino acid 226 of the heavy chain (by sequential numbering), for example amino acid 226 of SEQ ID NO:36.

In one embodiment the present disclosure provides a Fab′PEG molecule comprising one or more PEG polymers, for example 1 or 2 polymers such as a 40 kDa polymer or polymers.

In one embodiment there is provided a Fab′ conjugated to a polymer, such as a PEG molecule, a starch molecule or an albumin molecule.

In one embodiment there is provided a scFv conjugated to a polymer, such as a PEG molecule, a starch molecule or an albumin molecule.

In one embodiment the antibody or fragment is conjugated to a starch molecule, for example to increase the half life. Methods of conjugating starch to a protein as described in U.S. Pat. No. 8,017,739 incorporated herein by reference.

In one embodiment there is provided an anti-FcRn binding molecule which:
Causes 70% reduction of plasma IgG concentration,
With not more than 20% reduction of plasma albumin concentration, and/or
With the possibility of repeat dosing to achieve long-term maintenance of low plasma IgG concentration.

The present disclosure also provides an isolated DNA sequence encoding the heavy and/or light chain(s) of an antibody molecule of the present invention. Suitably, the DNA sequence encodes the heavy or the light chain of an antibody molecule of the present invention. The DNA sequence of the present invention may comprise synthetic DNA, for instance produced by chemical processing, cDNA, genomic DNA or any combination thereof.

DNA sequences which encode an antibody molecule of the present invention can be obtained by methods well known to those skilled in the art. For example, DNA sequences coding for part or all of the antibody heavy and light chains may be synthesised as desired from the determined DNA sequences or on the basis of the corresponding amino acid sequences.

DNA coding for acceptor framework sequences is widely available to those skilled in the art and can be readily synthesised on the basis of their known amino acid sequences.

Standard techniques of molecular biology may be used to prepare DNA sequences coding for the antibody molecule of the present invention. Desired DNA sequences may be synthesised completely or in part using oligonucleotide synthesis techniques. Site-directed mutagenesis and polymerase chain reaction (PCR) techniques may be used as appropriate.

Examples of suitable DNA sequences are provided in herein.

Examples of suitable DNA sequences encoding the 1519 light chain variable region are provided in SEQ ID NO:16, SEQ ID NO:17 and SEQ ID NO:90. Examples of suitable DNA sequences encoding the 1519 heavy chain variable region are provided in SEQ ID NO:30, SEQ ID NO:31 and SEQ ID NO:92.

Examples of suitable DNA sequences encoding the 1519 light chain (variable and constant) are provided in SEQ ID NO:23, SEQ ID NO:75 and SEQ ID NO:91.

Examples of suitable DNA sequences encoding the 1519 heavy chain (variable and constant, depending on format) are provided in SEQ ID NOs:37, 38 and 76 (Fab′), SEQ ID NO:72 or 85 (IgG1), SEQ ID NO: 44 or 93 (IgG4P) and SEQ ID:88 (IgG4).

Accordingly in one example the present disclosure provides an isolated DNA sequence encoding the heavy chain of an antibody Fab′ fragment of the present invention which comprises the sequence given in SEQ ID NO:37. Also provided is an isolated DNA sequence encoding the light chain of an antibody Fab′ fragment of the present invention which comprises the sequence given in SEQ ID NO:23.

In one example the present disclosure provides an isolated DNA sequence encoding the heavy chain and the light chain of an IgG4(P) antibody of the present invention in which the DNA encoding the heavy chain comprises the sequence given in SEQ ID NO:44 or SEQ ID NO:93 and the DNA encoding the light chain comprises the sequence given in SEQ ID NO:75 or SEQ ID NO:91.

In one example the present disclosure provides an isolated DNA sequence encoding the heavy chain and the light chain of a Fab-dsFv antibody of the present invention in which the DNA encoding the heavy chain comprises the sequence given in SEQ ID NO:51 or SEQ ID NO:80 and the DNA encoding the light chain comprises the sequence given in SEQ ID NO:47 or SEQ ID NO:79.

The present invention also relates to a cloning or expression vector comprising one or more DNA sequences of the present invention. Accordingly, provided is a cloning or expression vector comprising one or more DNA sequences encoding an antibody of the present invention. Suitably, the cloning or expression vector comprises two DNA sequences, encoding the light chain and the heavy chain of the antibody molecule of the present invention, respectively and suitable signal sequences. In one example the vector comprises an intergenic sequence between the heavy and the light chains (see WO03/048208).

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding an antibody of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells.

Suitable types of Chinese Hamster Ovary (CHO cells) for use in the present invention may include CHO and CHO-K1 cells including dhfr-CHO cells, such as CHO-DG44 cells and CHO-DXB11 cells and which may be used with a DHFR selectable marker or CHOK1-SV cells which may be used with a glutamine synthetase selectable marker. Other cell types of use in expressing antibodies include lymphocytic cell lines, e.g., NSO myeloma cells and SP2 cells, COS cells.

The present disclosure also provides a process for the production of an antibody molecule according to the present invention comprising culturing a host cell containing a vector of the present invention under conditions suitable for leading to expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

The antibodies and fragments according to the present disclosure are expressed at good levels from host cells. Thus the properties of the antibodies and/or fragments are conducive to commercial processing.

Thus there is a provided a process for culturing a host cell and expressing an antibody or fragment thereof, isolating the latter and optionally purifying the same to provide an isolated antibody or fragment. In one embodiment the process further comprises the step of conjugating an effector molecule to the isolated antibody or fragment, for example conjugating to a PEG polymer in particular as described herein.

In one embodiment there is provided a process for purifiying an antibody (in particular an antibody or fragment according to the invention) comprising the steps: performing anion exchange chromatography in non-binding mode such that the impurities are retained on the column and the antibody is eluted.

In one embodiment the purification employs affinity capture on an FcRn column.

In one embodiment the purification employs cibacron blue or similar for purification of albumin fusion or conjugate molecules.

Suitable ion exchange resins for use in the process include Q.FF resin (supplied by GE-Healthcare). The step may, for example be performed at a pH about 8.

The process may further comprise an initial capture step employing cation exchange chromatography, performed for example at a pH of about 4 to 5, such as 4.5. The cation exchange chromatography may, for example employ a resin such as CaptoS resin or SP sepharose FF (supplied by GE-Healthcare). The antibody or fragment can then be eluted from the resin employing an ionic salt solution such as sodium chloride, for example at a concentration of 200 mM.

Thus the chromatograph step or steps may include one or more washing steps, as appropriate.

The purification process may also comprise one or more filtration steps, such as a diafiltration step.

Thus in one embodiment there is provided a purified anti-FcRn antibody or fragment, for example a humanised antibody or fragment, in particular an antibody or fragment according to the invention, in substantially purified from, in particular free or substantially free of endotoxin and/or host cell protein or DNA.

Purified form as used supra is intended to refer to at least 90% purity, such as 91, 92, 93, 94, 95, 96, 97, 98, 99% w/w or more pure.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 μg per mg of antibody product or less such as 100 μg per mg or less, in particular 20 μg per mg, as appropriate.

As the antibodies of the present invention are useful in the treatment and/or prophylaxis of a pathological condition, the present invention also provides a pharmaceutical or diagnostic composition comprising an antibody molecule of the present invention in combination with one or more of a pharmaceutically acceptable excipient, diluent or carrier. Accordingly, provided is the use of an antibody molecule of the invention for the manufacture of a medicament. The composition will usually be supplied as part of a sterile, pharmaceutical composition that will normally include a pharmaceutically acceptable carrier. A pharmaceutical composition of the present invention may additionally comprise a pharmaceutically-acceptable excipient.

The present disclosure also provides a process for preparation of a pharmaceutical or diagnostic composition comprising adding and mixing the antibody molecule of the present invention together with one or more of a pharmaceutically acceptable excipient, diluent or carrier.

The antibody molecule may be the sole active ingredient in the pharmaceutical or diagnostic composition or may be accompanied by other active ingredients including other antibody ingredients or non-antibody ingredients such as steroids or other drug molecules, in particular drug molecules whose half-life is independent of FcRn binding.

The pharmaceutical compositions suitably comprise a therapeutically effective amount of the antibody of the invention. The term “therapeutically effective amount” as used herein refers to an amount of a therapeutic agent needed to treat, ameliorate or prevent a targeted disease or condition, or to exhibit a detectable therapeutic or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans. Suitable doses are provided herein below.

Pharmaceutical compositions may be conveniently presented in unit dose forms containing a predetermined amount of an active agent of the invention per dose.

Therapeutic doses of the antibodies according to the present disclosure show no apparent toxicology effects in vivo.

In one embodiment of an antibody or fragment according to the invention a single dose may provide up to a 70% reduction in circulating IgG levels.

In one example of the method or use of the present invention, the maximal reduction (nadir) in circulating IgG levels may be up to a 40%, up to a 50%, up to a 60% or up to a 70% reduction.

The maximal therapeutic reduction in circulating IgG may be observed about 1 week after administration of the relevant therapeutic dose. The levels of IgG may recover over about a six week period if further therapeutic doses are not delivered. Alternatively, the maximal therapeutic reduction in circulating IgG may be observed about 2, 3, 4, 5 or 6 weeks after administration of the relevant therapeutic dose(s).

Advantageously, the levels of IgG in vivo may be maintained at an appropriately low level by administration of sequential doses of the antibody or fragments according to the disclosure.

Compositions may be administered individually to a patient or may be administered in combination (e.g. simultaneously, sequentially or separately) with other agents, drugs or hormones.

In one embodiment the antibodies or fragments according to the present disclosure are employed with an immunosuppressant therapy, such as a steroid, in particular prednisone.

In one embodiment the antibodies or fragments according to the present disclosure are employed with Rituximab or other B cell therapies.

In one embodiment the antibodies or fragments according to the present disclosure are employed with any B cell or T cell modulating agent or immunomodulator. Examples include methotrexate, microphenyolate and azathioprine.

The frequency of dose will depend on the half-life of the antibody molecule and the duration of its effect. If the antibody molecule has a short half-life (e.g. 2 to 10 hours) it may be necessary to give one or more doses per day. Alternatively, if the antibody molecule has a long half life (e.g. 2 to 15 days) and/or long lasting pharmacodynamics (PD) profile it may only be necessary to give a dosage once per day, once per week or even once every 1 or 2 months. Suitable dosage regimes are provided herein below.

In one embodiment the dose is delivered bi-weekly, i.e. twice a month.

Half life as employed herein is intended to refer to the duration of the molecule in circulation, for example in serum/plasma.

Pharmacodynamics as employed herein refers to the profile and in particular duration of the biological action of the molecule according the present disclosure.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

Suitably in formulations according to the present disclosure, the pH of the final formulation is not similar to the value of the isoelectric point of the antibody or fragment, for example if the pI of the protein is in the range 8-9 or above then a formulation pH of 7 may be appropriate. Whilst not wishing to be bound by theory it is thought that this may ultimately provide a final formulation with improved stability, for example the antibody or fragment remains in solution.

In one example the pharmaceutical formulation at a pH in the range of 4.0 to 7.0 comprises: 1 to 200 mg/mL of an antibody molecule according to the present disclosure, 1 to 100 mM of a buffer, 0.001 to 1% of a surfactant, a) 10 to 500 mM of a stabiliser, b) 10 to 500 mM of a stabiliser and 5 to 500 mM of a tonicity agent, or c) 5 to 500 mM of a tonicity agent.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. Dosage treatment may be a single dose schedule or a multiple dose schedule. Preferably the delivery is subcutaneous.

It will be appreciated that the active ingredient in the composition will be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, N.J. 1991).

The antibody of the invention can be delivered dispersed in a solvent, e.g., in the form of a solution or a suspension. It can be suspended in an appropriate physiological solution, e.g., saline or other pharmacologically acceptable solvent or a buffered solution. A suspension can employ, for example, lyophilised antibody.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent/solution used for the formulation, aseptic suspension of the antibody in the sterile buffered solvent solution, and dispensing of the formulation into sterile receptacles by methods familiar to those of ordinary skill in the art.

Once formulated, the compositions of the disclosure can be administered directly to the human subject, although the disclosure also contemplates that the method may be employed on non-human subjects. The human, in some aspects of the disclosure, suffers from primary persistent or chronic primary ITP. Recently, new definitions for the phases of the disease were introduced (Rodeghiero et al, 2009, Blood. 113(11):2386-93). Based on the time from diagnosis, the first phase (up to 3 months) is the “newly-diagnosed ITP,” phase 2 (>3 months up to 12 months) is the “persistent phase,” and after 12 months the “chronic phase” starts. These phases also reflect the line of treatment.

The major goal for treatment in primary ITP is to provide a sufficient platelet count to prevent or stop bleeding rather than correcting the platelet count to normal levels. In chronic ITP the goal of treatment is also to avoid or defer the risks of more toxic treatments (e.g. splenectomy or immunosuppression), reduce corticosteroid exposure to minimum levels and for the shortest time and achieve long-lasting responses. On-demand treatment at the time of or in anticipation of high risk bleeding or surgical procedures is another approach that is often warranted.

Accordingly, in one example, the method of treatment of the present invention may be used for the on-demand treatment of ITP.

Autoantibodies involved in the pathogenesis of ITP may include both IgG and non-IgG isotypes. In one example, the method of treatment of the present disclosure may be used for the treatment of ITP patients where IgG is determined to be the dominant isotype.

Efficacy of treatment for ITP is considered to be achievement of a platelet count that prevents major bleeding rather than correcting the platelet count to normal levels. Accordingly, the management of ITP should be tailored to the individual patient and it is rarely indicated in those with platelet counts above 50×10⁹/L in the absence of bleeding, trauma, surgery, or high risk factors (eg, patients on anticoagulation therapy) (EMA/CHMP/153191/2013, 2014).

However, there is general agreement that typically adults with a platelet count of <30×10⁹/L with bleeding at diagnosis require treatment.

### Administration Regimens

The composition preferably comprises a therapeutically effective amount of the antibody (or antigen binding fragment thereof). The term “therapeutically effective amount” as used herein refers to an amount of a therapeutic agent needed to treat, ameliorate or prevent a targeted disease or condition, or to exhibit a detectable therapeutic or preventative effect. “Administration regimen” contemplates the amount (dose) of antibody or fragment thereof administered as well as timing of administration if multiple doses are provided.

Several methods of characterizing ITP disease and treatment efficacy exist and are appropriate for detecting a positive biological response in the subject.

One assessment is the platelet count.

Another helpful assessment of ITP is the ITP bleeding score.

The International Working Group on ITP now proposes a consensus-based ITP-specific Bleeding Assessment Tool (ITP-BAT), based on a precise definition of bleeding manifestations and on the grading of their severity (Rodeghiero et al, 2013, Standardization of bleeding assessment in immune thrombocytopenia: report from the International Working Group. Blood. 121(14):2596-606.). The ITP bleeding score may be assessed using the ITP-BAT tool version 1.0.

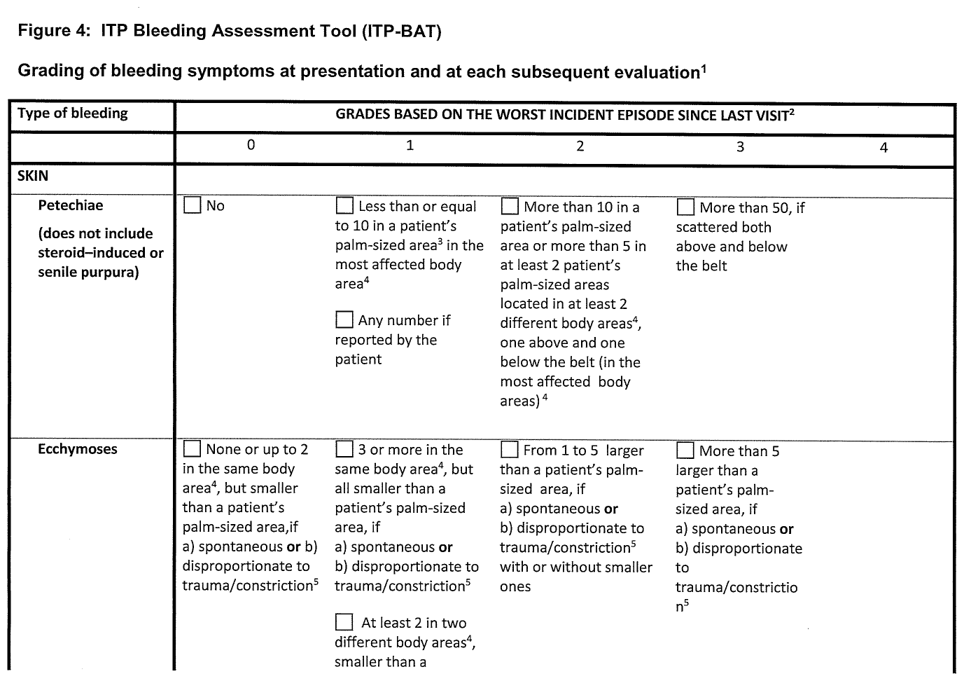

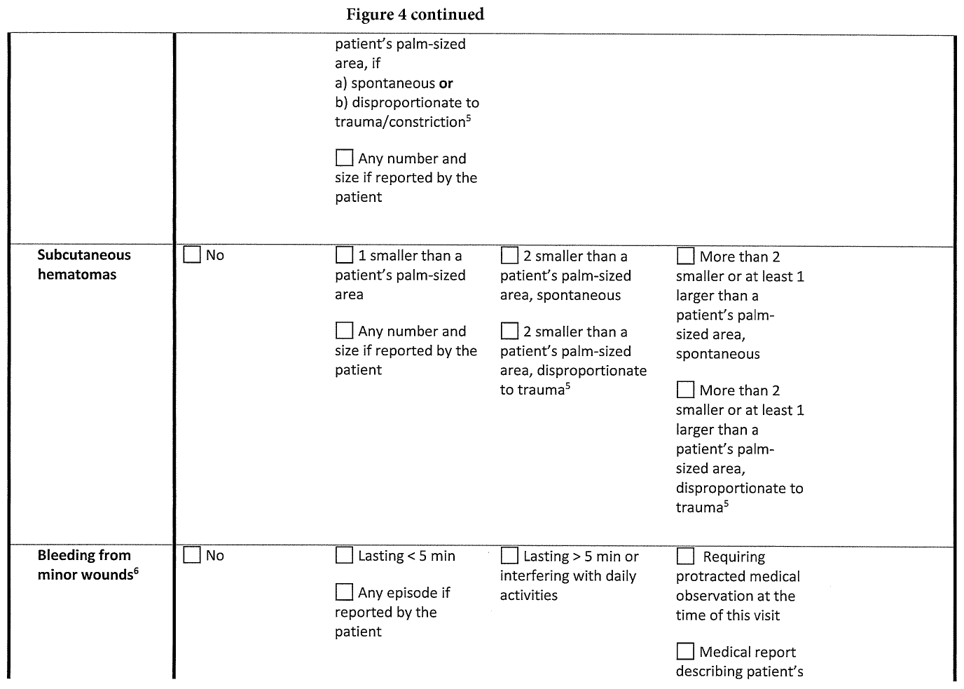

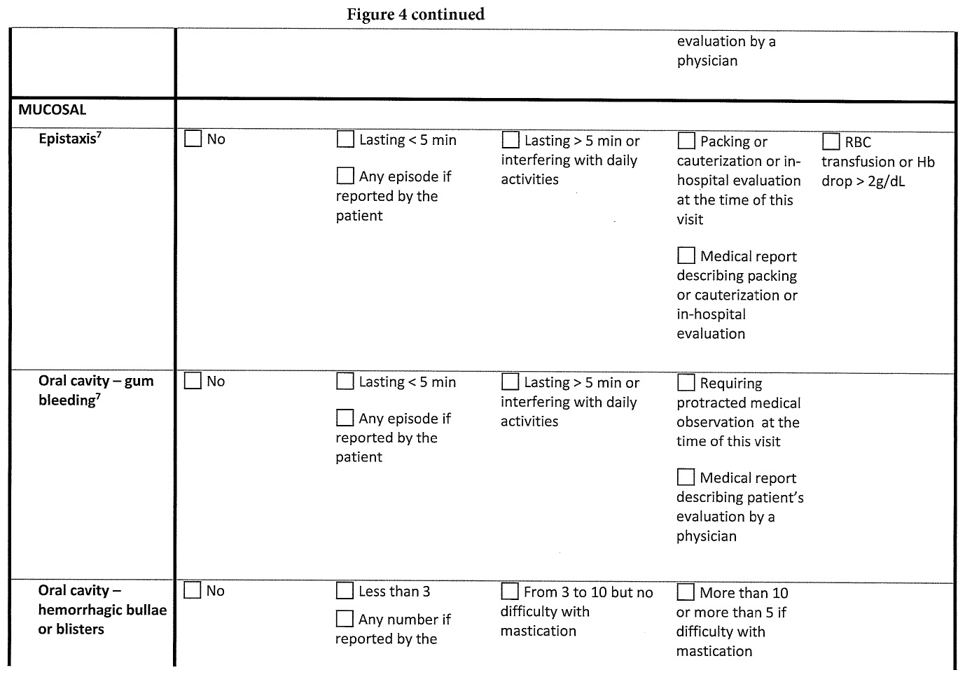

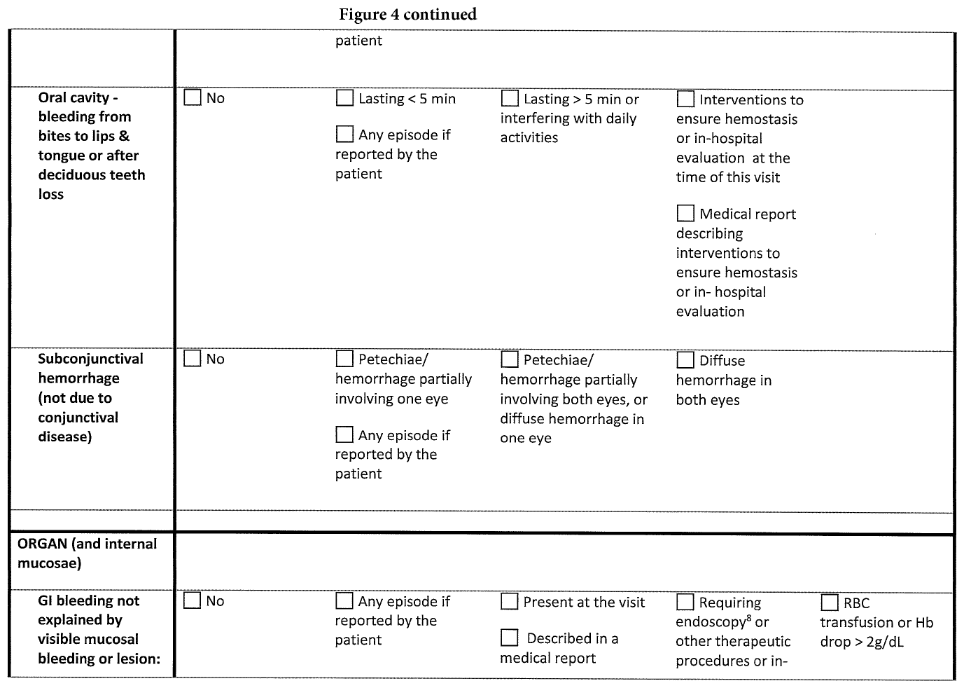

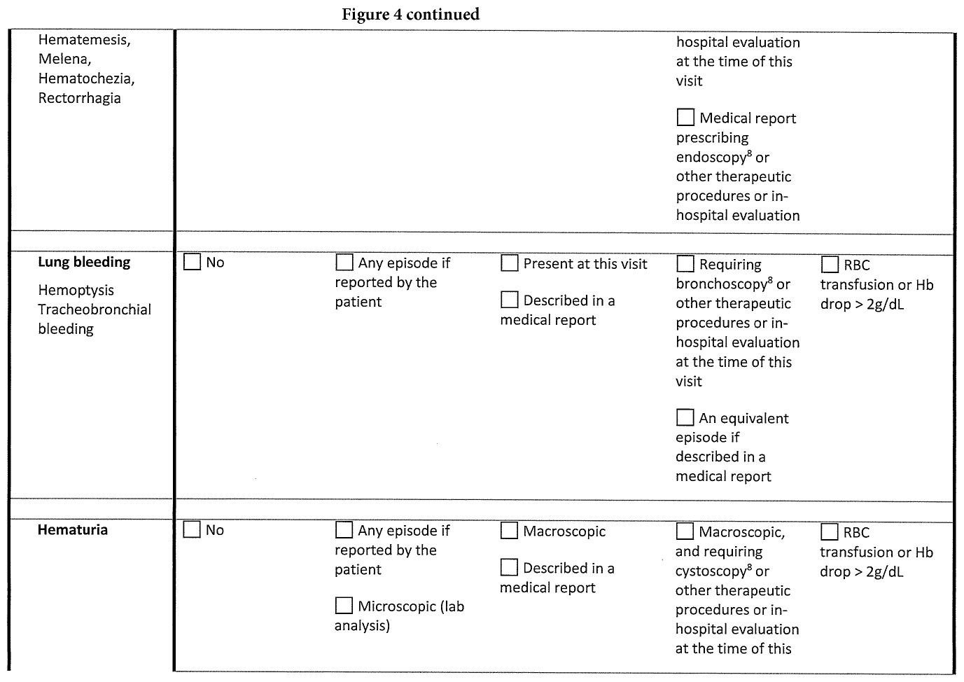

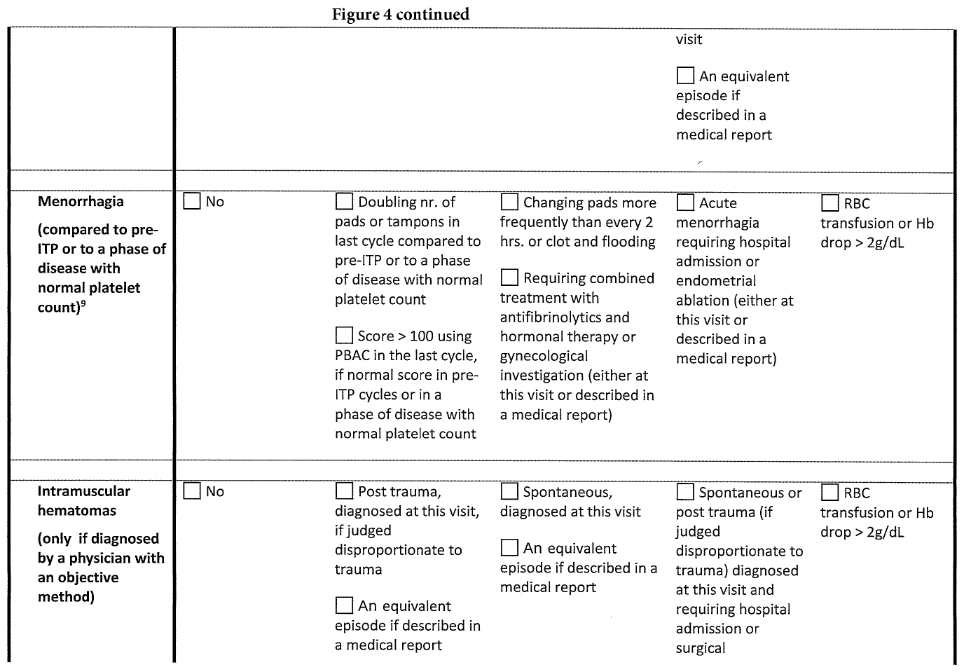

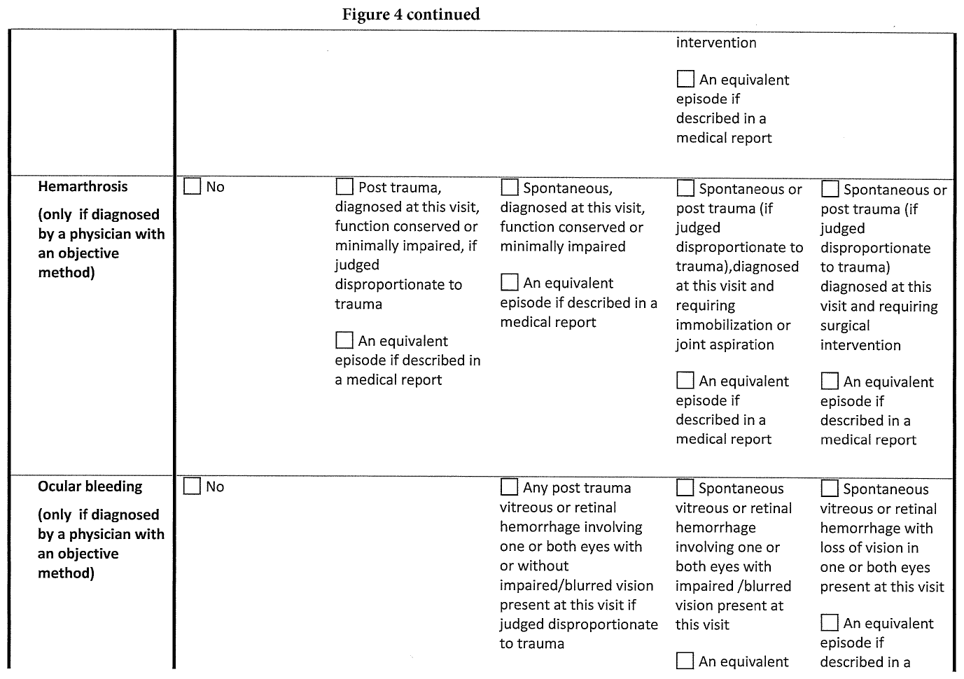

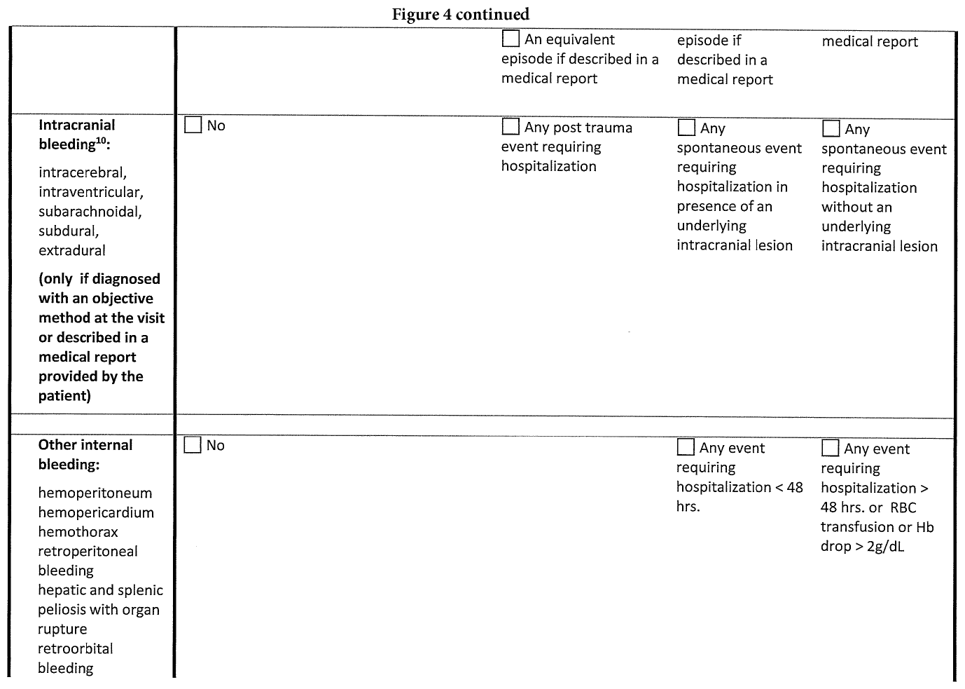

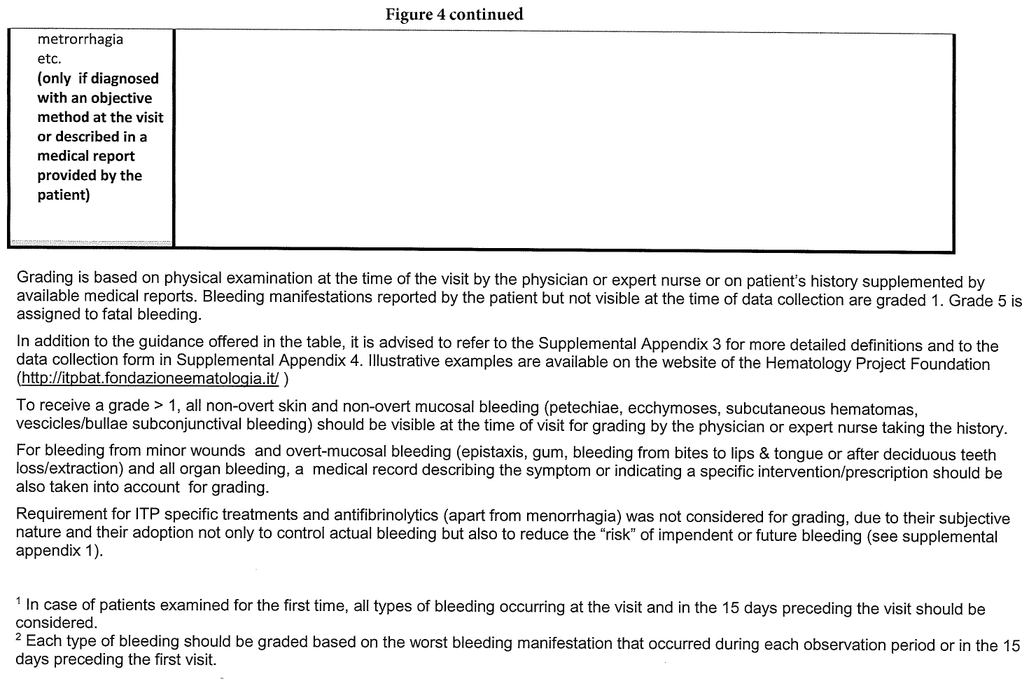

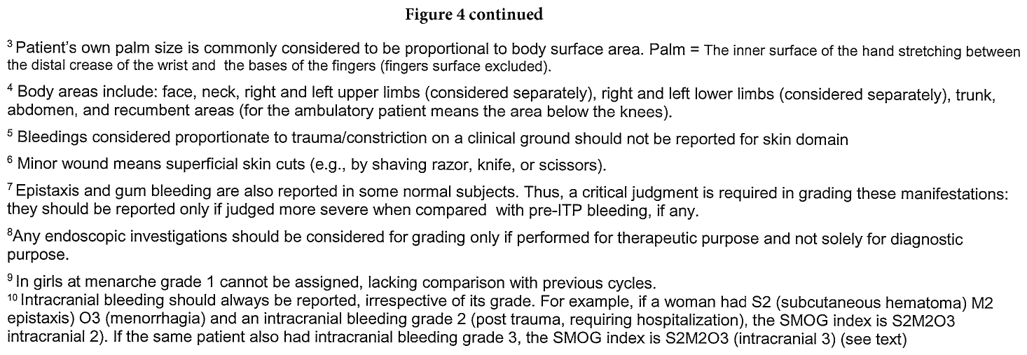

For the ITP-BAT, bleeding manifestations were grouped into 3 major domains: skin (S), visible mucosae (M), and organs (0), with gradation of severity (SMOG). Each bleeding manifestation is assessed at the time of examination. Severity is graded from 0 to 3 or 4, with grade 5 for any fatal bleeding. Bleeding reported by the subject without medical documentation is graded 1. Within each domain, the same grade is assigned to bleeding manifestations of similar clinical impact. The “worst” bleeding manifestation since the last Observation Period visit is graded, and the highest grade within each domain is recorded. The SMOG system provides a consistent description of the bleeding phenotype in ITP, see FIG. 4.

Absence of bleeding is indicated by Grade 0 for all domains of the SMOG. Presence of bleeding is indicated by a Grade of 1 or above, for at least one domain of the SMOG.

In one example efficacy (clinical response) is defined as a platelet count ≥30×10⁹/L and at least 2-fold increase of the Baseline count.

In one example, efficacy (clinical response) is defined as platelet count ≥30×10⁹/L and at least 2-fold increase from Baseline value and absence of bleeding

In one example efficacy (clinical response) is defined as a platelet count ≥50×10⁹/L In one example efficacy (clinical response) is defined as a platelet count ≥50×10⁹/L and absence of bleeding.

In one example efficacy (complete clinical response) is defined as a platelet count of ≥100×10⁹/L.

In one example efficacy (complete clinical response) is defined as a platelet count of ≥100×10⁹/L and absence of bleeding.

In one example a clinical response is defined once a platelet count as described above is confirmed on 2 separate occasions at least 7 days apart (ie, the second assessment should be ≥168 hours after the first assessment).

As used herein, “treating” and “treatment” refers to any reduction in the severity of ITP and “preventing” or “prevention” refers to any reduction or delay in the onset of symptoms of ITP. One of ordinary skill in the art will appreciate that any degree of protection from, or amelioration of, ITP or symptom associated therewith is beneficial to a subject, such as a human patient. The quality of life of a patient is improved by reducing to any degree the severity of symptoms in a subject and/or delaying the appearance of symptoms. Accordingly, the method in one aspect is performed as soon as possible after it has been determined that a subject is suffering from or at risk of suffering from ITP.

The major goal for treatment in primary ITP is to provide a sufficient platelet count to prevent or stop bleeding rather than correcting the platelet count to normal levels. In chronic ITP the goal of treatment is also to avoid or defer the risks of more toxic treatments (e.g. splenectomy or immunosuppression), reduce corticosteroid exposure to minimum levels and for the shortest time and achieve long-lasting responses. On-demand treatment at the time of or in anticipation of high risk bleeding or surgical procedures is another approach that is often warranted.

In various aspects, the antibody or fragment thereof is administered via an administration regimen that achieves an improvement in platelet count at, e.g., one, two, three, four, five, six, seven, eight, nine, ten, eleven, or twelve weeks following initial administration of the antibody or fragment thereof.

Alternatively or in addition, the antibody or fragment thereof is administered via an administration regimen that achieves an improvement in platelet count and ITP bleeding score compared to pre-treatment. Preferably the improvement in platelet count and ITP bleeding score is observed at, e.g., one, two, three, four, five, six, seven, eight, nine, ten, eleven, or twelve weeks following initial administration of the antibody or fragment thereof.

Improvements are as set out above for clinical response, for example an improvement may be an increase in platelet count to ≥30×10⁹/L and at least 2-fold increase of the Baseline count.

Alternatively an improvement may be an increase in platelet count to ≥30×10⁹/L and at least 2-fold increase from Baseline value and absence of bleeding

Alternatively an improvement may be an increase in platelet count to ≥50×10⁹/L Alternatively an improvement may be an increase in platelet count to ≥50×10⁹/L and absence of bleeding.

Alternatively an improvement may be an increase in platelet count to ≥100×10⁹/L.

Alternatively an improvement may be an increase in platelet count to ≥100×10⁹/L and absence of bleeding.

In one example an improvement is defined once a platelet count as described above is confirmed on 2 separate occasions at least 7 days apart (ie, the second assessment should be ≥168 hours after the first assessment).

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Generally, a therapeutically effective amount will be from 0.01 mg/kg to 500 mg/kg, preferably 0.1 mg/kg to 30 mg/kg (e.g., 4 mg/kg to 25 mg/kg, such as about 10 mg/kg, 20 mg/kg or 21 mg/kg). Compositions may be conveniently presented in unit dose forms containing a predetermined amount of an active agent of the disclosure per dose. Dose ranges and regimens for any of the embodiments described herein include, but are not limited to, dosages ranging from 1 mg-100 mg unit doses (e.g., 4 mg, 7 mg, 10 mg, 15 mg, 20 mg, 25 mg or 30 mg), or 100-200 mg unit doses, such as 100 mg, 140 mg, 160 mg unit doses given every 1-10 weeks (by any route of administration, such as by as either a subcutaneous or intravenous administration).

Optionally, a dose of antibody is administered every 1-20 weeks, for example every week, every 2 weeks, every 3 weeks, every 4 weeks, every 5 weeks, every 6 weeks, every 7 weeks, or every 8-12 weeks (e.g., every 8 weeks, every 9 weeks, every 10 weeks, every 11 weeks, or every 12 weeks). The treatment period (i.e., the period of time during which one or more doses of antibody are administered to a subject) may comprise at least 1 week, at least two weeks, at least 3 weeks, at least four weeks, at least 5 weeks, at least six weeks, at least seven weeks, at least eight weeks or more. Any suitable number of doses may be administered within the treatment period, such as the doses and time between administrations described above. For example, one, two, three, four, or five doses of antibody are administered to a subject over, e.g., a 5 week treatment period (optionally at week 0 and every week thereafter (week 1, week 2, week 3 and week 4) or a 3 week treatment period (optionally at week 0 and every week thereafter (week 1 and week 2).

Thus in one aspect there is provided a method of treating or preventing immune (idiopathic) thrombocytopenia (ITP) in a human in need thereof, the method comprising administering to the human in the range of 1 to 5 doses of an anti-FcRn antibody or antigen binding fragment thereof over a treatment period of 1 to 12 weeks, wherein the aggregrate dose in the treatment period is in the range of 1 to 30 mg per kg.

It will be appreciated that the therapeutically effective amount may be delivered in a single dose or in multiple doses over a given treatment period. For example, a dose of 20 mg/kg may be given as a single dose or as 5 doses of 4 mg/kg.

In one example each dose is 4 mg/kg, for example administered as five individual doses, in particular over a treatment period of five weeks, in particular five consecutive weeks.

In one example each dose is 7 mg/kg, for example administered as three individual doses, in particular over a treatment period of three weeks, in particular three consecutive weeks.

In one example each dose is 10 mg/kg, for example administered as two individual doses, in particular over a treatment period of two weeks, in particular two consecutive weeks.

In one example a single dose is administered, for example a single dose of 15 mg/kg.

In one example the single dose is 20 mg/kg.

In one example the single dose is 25 mg/kg

In one example the single dose is 30 mg/kg

In one example, the aggregate dose (i.e. the total dose given over the treatment period) is selected from 10, 15, 20, 21, 25 and 30 mg/kg.

In one example each dose is 4 mg/Kg, for example administered as five individual doses, in particular over a treatment period of five weeks, in particular five consecutive weeks, resulting in an aggregate dose of 20 mg/Kg.

In one example each dose is 7 mg/Kg, for example administered as three individual doses, in particular over a treatment period of three weeks, in particular three consecutive weeks, resulting in an aggregate dose of 21 mg/Kg.

In one example each dose is 10 mg/Kg, for example administered as two individual doses, in particular over a treatment period of two weeks, in particular two consecutive weeks, resulting in an aggregate dose of 20 mg/Kg.

In one example each dose is 15 mg/Kg, for example administered as two individual doses, in particular over a treatment period of two weeks, in particular two consecutive weeks, resulting in an aggregate dose of 30 mg/Kg.

Optionally, the method employs a repeat dose administration strategy with different dosing regimens involving a higher initial dose (i.e., a “loading dose”) followed by one or more lower doses (i.e., one or more second or additional doses that are lower than the initial dose (“maintenance doses”)), although a lower loading dose followed by higher maintenance doses also are contemplated. In one embodiment, the maintenance doses may be one-quarter, one-third, one-half, two-thirds, three-quarters, the same as, one and one-quarter, one and one-third, one and one-half, one and two-thirds, one and three-quarters, double, or more of the loading dose. A multiple dose regimen without a loading dose also is contemplated as part of the disclosure.

The frequency of dose will depend on the half-life of the antibody molecule and the duration of its effect. If the antibody molecule has a short half-life (e.g., 2 to 10 hours) it may be necessary to give one or more doses per day. Alternatively, if the antibody molecule has a long half-life (e.g., 2 to 15 days) it may only be necessary to give a dosage once per day, once per week or even once every 1 or 2 months. Alternatively, or in addition, frequency of dosing may be determined by disease severity, determined for example by disease biomarker monitoring and/or by monitoring patient platelet levels and/or serum IgG levels.

In one embodiment, one or more maintenance doses are administered at an interval after administration of a loading dose. This interval may be consistent for each dose or may vary. This interval may be 1 day, 1 week, 2 weeks, 3 weeks, 4 weeks, monthly, 6 weeks, 8 weeks, every other month, or at any other interval. In one embodiment, five maintenance doses are administered every two weeks after a loading dose for a total of six doses.

Accordingly, in one example, the method of treatment of the present invention further comprises administering one or more second or additional doses that are lower than the initial dose(s).

It will be appreciated that these additional doses may be administered beyond the initial treatment period for the higher ‘loading dose’.

Accordingly, in one example, the higher initial aggregate dosing of 20-30 mg over a treatment period of preferably 1-5 weeks, may be followed by a further maintenance dosing treatment period.

In one example, the present invention provides a method of treating or preventing immune (idiopathic) thrombocytopenia (ITP) in a human in need thereof, the method comprising administering to the human in the range of 1 to 5 doses of an anti-FcRn antibody or antigen binding fragment thereof over a treatment period of 1 to 12 weeks, wherein the aggregrate dose in the treatment period is in the range of 1 to 30 mg per kg, followed by one or more second or additional doses that are lower than the initial doses.

In one example, the present invention provides a method of treating or preventing immune (idiopathic) thrombocytopenia (ITP) in a human in need thereof, the method comprising administering to the human in the range of 1 to 5 doses of an anti-FcRn antibody or antigen binding fragment thereof over a treatment period of 1 to 5 weeks, wherein the aggregrate dose in the treatment period is in the range of 20 to 30 mg per kg, followed by one or more second or additional doses that are lower than the initial dose(s).

In one example, the present invention provides a method of treating or preventing immune (idiopathic) thrombocytopenia (ITP) in a human in need thereof, the method comprising administering to the human 1 or 2 doses of an anti-FcRn antibody or antigen binding fragment thereof over a treatment period of 1 or 2 weeks, wherein the aggregrate dose in the treatment period is 20 mg per kg or 30 mg/Kg, optionally followed by one or more second or additional doses that are lower than the initial dose (maintenance doses).

In one example each dose is 10 mg/kg, for example administered as two individual doses over a treatment period of two weeks, in particular two consecutive weeks, optionally followed by one more additional doses that are lower than 10 mg/Kg.

In one example each dose is 15 mg/kg, for example administered as two individual doses over a treatment period of two weeks, in particular two consecutive weeks, optionally followed by one more additional doses that are lower than 15 mg/Kg.

In one example a single dose of 15 mg/kg, is administered over a treatment period of 1 week, optionally followed by one more additional doses that are lower than 15 mg/Kg.

In one example a single dose of 20 mg/kg, is administered over a treatment period of 1 week, optionally followed by one more additional doses that are lower than 20 mg/Kg.

In one example, each lower (“maintenance”) dose is between 4 and 10 mg/Kg, preferably 4 or 7 mg/Kg.

As set out above, these doses may be given at any suitable interval such as 1 week, 2 weeks, 3 weeks, 4 weeks, monthly, 6 weeks, 8 weeks, every other month, or at any other interval.

In one example a maintenance dose is given at an interval of every week.

In one example a maintenance dose is given at an interval of every 2 weeks.

In one example a maintenance dose is given at an interval of every 4 weeks.

In one example a maintenance dose is given monthly.

In one example the higher initial dose (loading dose) comprises treatment with 5 doses of 4 mg/Kg or 3 doses of 7 mg/Kg at weekly intervals over a treatment period of 5 or 3 weeks (aggregate dose of 20 and 21 mg/kg respectively) and maintenance dosing may comprise dosing at 4 mg/Kg or 7 mg/Kg at a suitable interval such every 2 weeks, 3 weeks, 4 weeks, monthly, 6 weeks, 8 weeks, every other month, or at any other interval.

The method of treatment of the present invention may be suitable for both on-demand treatment and/or maintenance therapy for ITP.

Timing between administrations may decrease as the condition improves or increase if it worsens, reverting to the higher doses as needed for acute episodes.

Timing of administration may also be determined by monitoring patient platelet levels and/or serum IgG levels.

In one example, the method of treatment of the present invention may be used for the on-demand treatment of ITP.

Autoantibodies involved in the pathogenesis of ITP may include both IgG and non-IgG isotypes. In one example, the method of treatment of the present disclosure may be used for the treatment of ITP patients where IgG is determined to be the dominant isotype.

In one aspect there is provided a method of treating or preventing immune (idiopathic) thrombocytopenia (ITP) in a human in need thereof, the method comprising administering to the human in the range of 1 to 5 doses of an anti-FcRn antibody or antigen binding fragment thereof over a treatment period of 1 to 12 weeks, wherein the aggregrate dose in the treatment period is in the range of 1 to 40 mg per kg.

In one example each dose is 20 mg/Kg, for example administered as two individual doses, in particular over a treatment period of two weeks, in particular two consecutive weeks, resulting in an aggregate dose of 40 mg/Kg.

In one example each dose is 20 mg/Kg, for example administered as two individual doses, in particular over a treatment period of two weeks, in particular two consecutive weeks, resulting in an aggregate dose of 40 mg/Kg optionally followed by one more additional doses that are lower than 20 mg/Kg.

Comprising in the context of the present specification is intended to meaning including. Where technically appropriate embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

The present invention is further described by way of illustration only in the following examples, which refer to the accompanying Figures, in which:

FIG. 1 shows certain amino acid and polynucleotide sequences.

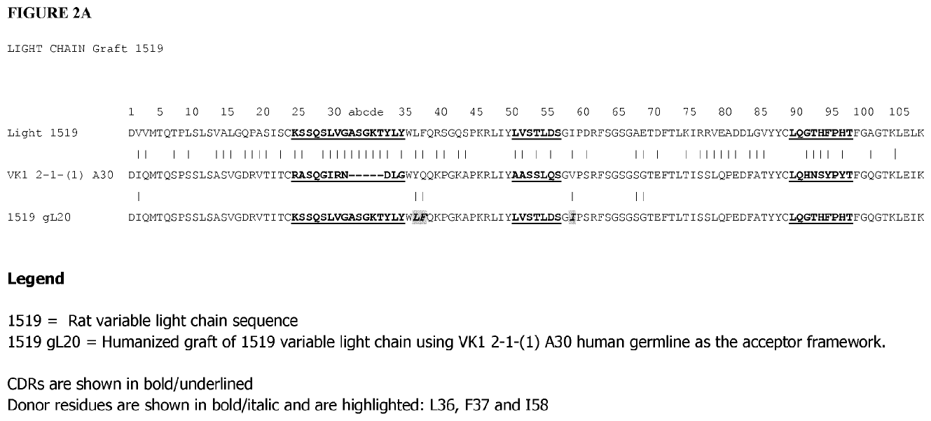

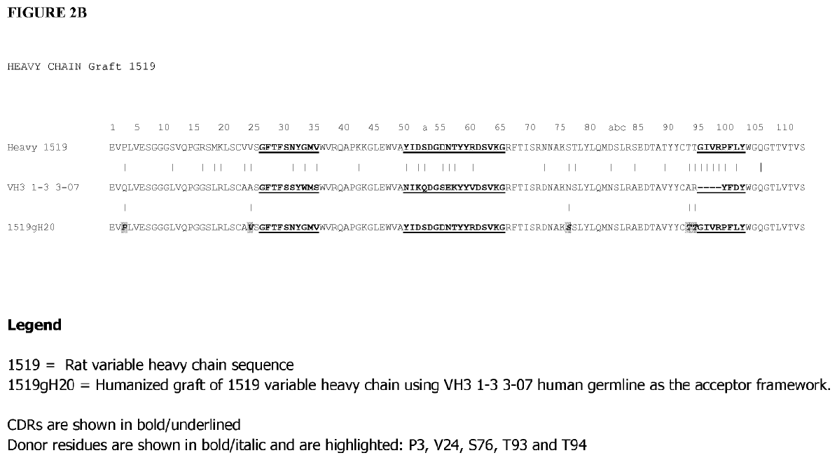

FIG. 2 shows alignments of certain sequences.

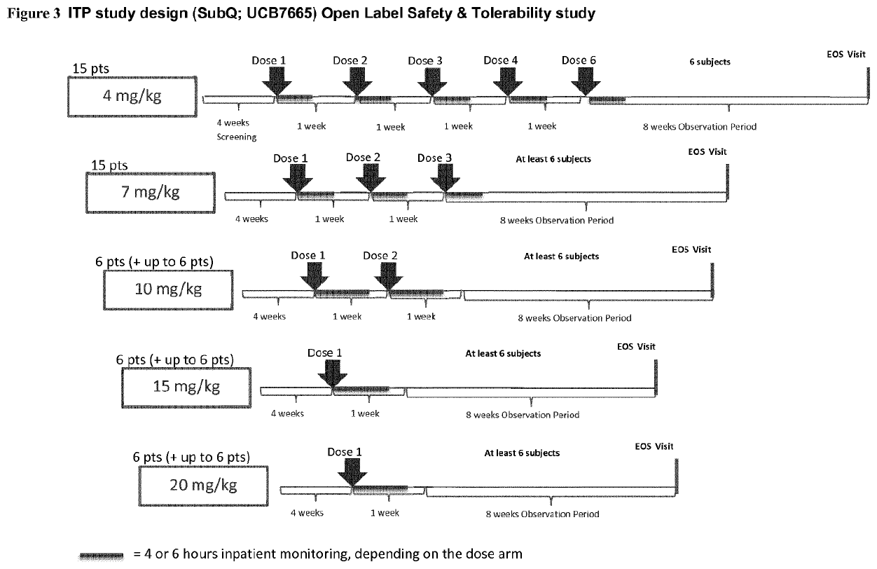

FIG. 3 shows ITP study design (SubQ; UCB7665) Open Label Safety & Tolerability study

FIG. 4 ITP Bleeding Assessment Tool (ITP-BAT)

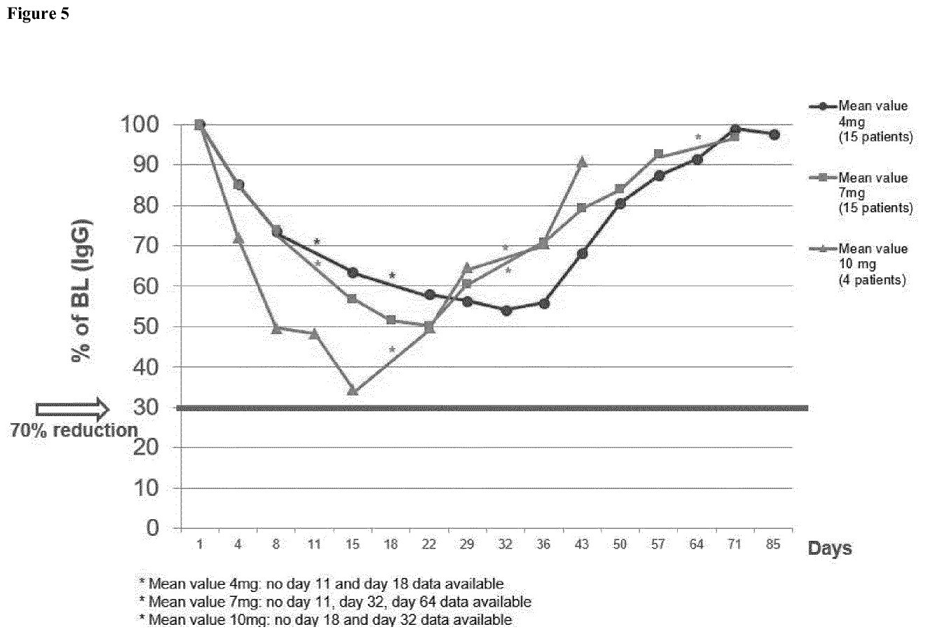

FIG. 5 Decrease from baseline (mean IgG reduction) following multiple UCB7665 4, 7 and 10 mg/kg administration

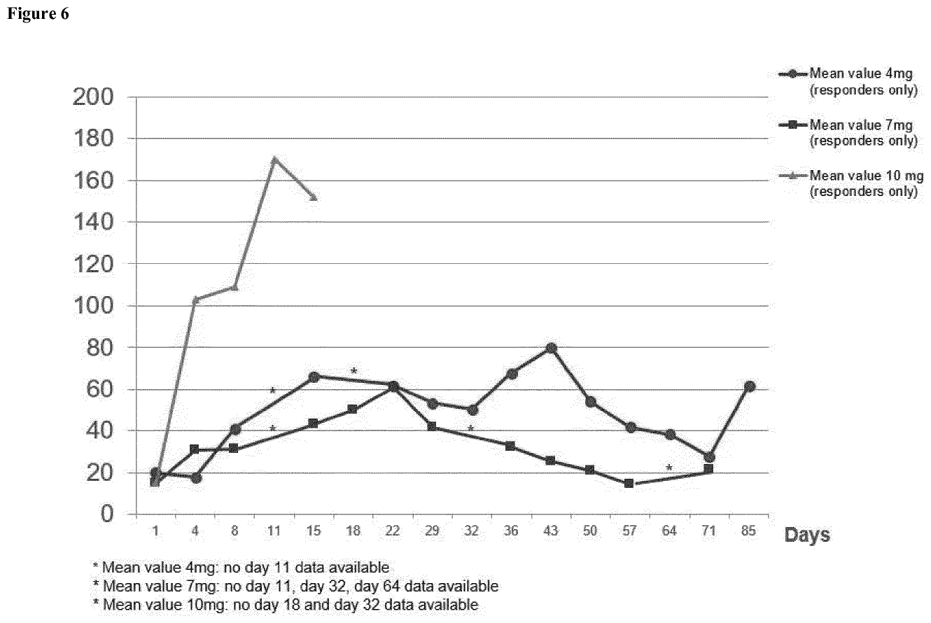

FIG. 6 Mean Platelet count following multiple UCB7665 4, 7 and 10 mg/kg administration (only responders)

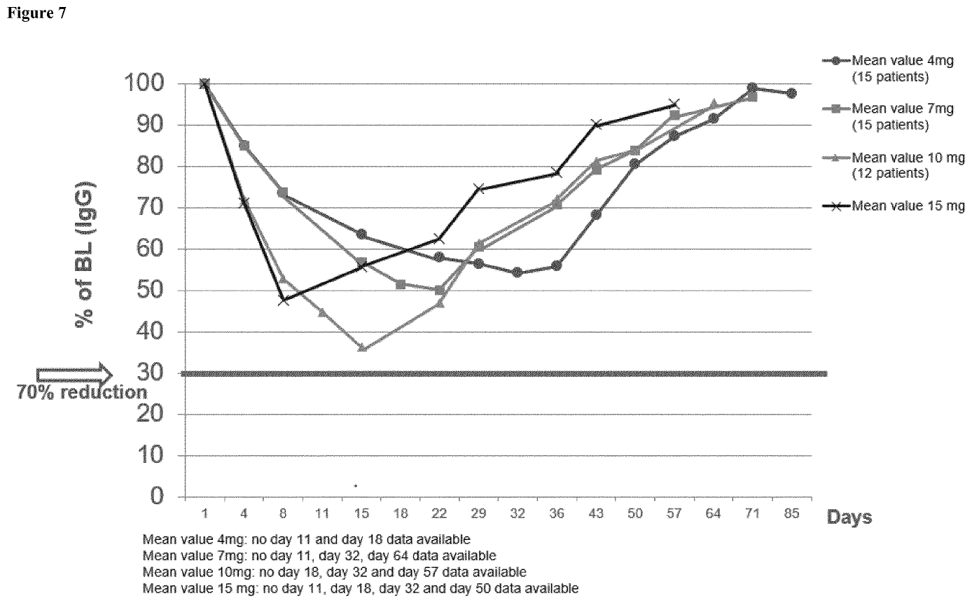

FIG. 7 Decrease from baseline (mean IgG reduction) following multiple UCB7665 4, 7, 10 and single 15 mg/kg administration

### EXAMPLES

### Example 1

UCB7665 was first described in WO2014019727 and comprises the CDR sequences provided herein in SEQ ID NOs 1-6. It comprises the light chain of SEQ ID NO:22 and the heavy chain of SEQ ID NO: 43.

UCB7665 has the INN rozanolixizumab.

Affinity for hFcRn Binding of UCB 7665 (Reproduced from WO2014019727)

Biomolecular interaction analysis using surface plasmon resonance technology (SPR) was performed on a Biacore T200 system (GE Healthcare) and binding to human FcRn extracellular domain determined. Human FcRn extracellular domain was provided as a non-covalent complex between the human FcRn alpha chain extracellular domain (SEQ ID NO:94) and β2 microglobulin (β2M) (SEQ ID NO:95). Affinipure F(ab′)2 fragment goat anti-human IgG, F(ab′)2 fragment specific (for Fab′-PEG capture) or Fc fragment specific (for IgG1 or IgG4 capture) (Jackson ImmunoResearch Lab, Inc.) in 10 mM NaAc, pH 5 buffer was immobilized on a CM5 Sensor Chip via amine coupling chemistry to a capture level between 4000-5000 response units (RU) using HBS-EP⁺ (GE Healthcare) as the running buffer. 50 mM Phosphate, pH6+150 mM NaCl+0.05% P20 or HBS-P, pH7.4 (GE Healthcare) was used as the running buffer for the affinity assay. The antibody was diluted to 4 g/ml (IgG4) in running buffer. A 60s injection of IgG4 at 10 l/min was used for capture by the immobilized anti-human IgG, F(ab′)2. Human FcRn extracellular domain was titrated from 20 nM to 1.25 nM over the captured anti-FcRn antibody for 300 s at 30 μl/min followed by 1200 s dissociation. The surface was regenerated by 2×60 s 50 mM HCl at 10l/min. The data was analysed using T200 evaluation software (version 1.0).

pH7.4

| 1519.g57 | ka (M⁻¹s⁻¹) | kd (s⁻¹) | KD (M) | KD (pM) |
|---|---|---|---|---|
| IgG4P | 3.68E+05 | 1.26E−05 | 3.43E−11 | 34 |

pH6

| 1519.g57 | ka (M⁻¹s⁻¹) | kd (s⁻¹) | KD (M) | KD (pM) |
|---|---|---|---|---|
| IgG4P | 4.43E+05 | 1.00E−05 | 2.26E−11 | 23 |

Affinity data for anti-hFcRn 1519.g57 IgG4P at pH7.4 and pH6
(average of three experiments)
Crystallography and Binding Epitope of UCB7665 (Reproduced from WO2014019727)

The crystal structure of 1519g57 Fab′ and deglycosylated human FcRn extracellular domain (alpha chain extracellular domain (SEQ ID NO:94) in association with beta2 microglobulin SEQ ID NO:95) was determined, with the FcRn oligsaccharide excluded in order to facilitate crystallization. 1519.g57 Fab′ was reacted with 10-fold molar excess of N-ethyl maleimide to prevent formation of diFab′ and any existing diFab′ removed by SEC (S200 on Akta FPLC). Human FcRn extracellular domain was treated by PNGaseF to remove N-linked sugars. For this, the FcRn sample concentration was adjusted using PBS (pH7.4) to 5 mg/ml and a total volume of 1 ml. 200 units of PNGaseF (Roche) was added to this solution of human FcRn. This was incubated at 37° C. for −18 hours, following which the extent of deglycosylation was checked using SDS PAGE. Upon completion of the reaction the deglycosylated FcRn was buffer exchanged into 50 mM Sodium Acetate, 125 mM NaCl, pH6.0.

The complex was formed by incubation of a mixture of reagents (Fab′:FcRn::1.2:1, w/w) at room temperature for 60 minutes, and then purified using SEC (S200 using Akta FPLC). Screening was performed using the various conditions that were available from Qiagen (approximately 2000 conditions). The incubation and imaging was performed by Formulatrix Rock Imager 1000 (for a total incubation period of 21 days).

There was no obvious change in FcRn structure upon binding of 1519g57 Fab′ (comparing this complex with published structures of FcRn). From the crystal structure it the secondary structure content was calculated to be: α-helix 9.4%; P3-sheet 45.2%; 3-10 turn 2.5%.

The residues interacting with 1519g57 Fab′ were all in the FcRn α chain (not (32M) and are indicated below in bold. The residues concerned encompass all but 1 of the residues critical for binding Fc. 1519g57 binds in a region that overlays the Fc-binding region, suggesting that blockade of FcRn by 1519g57 Fab′ is by simple competition, the anti-FcRn being effective by virtue of its superior affinity.

AESHLSLLYH LTAVSSPAPG TPAFWVSGWL GPQQYLSYNS
LRGEAEPCGA WVWENQVSWY WEKETTDLRI KEKLFLEAFK
ALGGKGPYTL QGLLGCELGP DNTSVPTAKF ALNGEEFMNF
DLKQGTWGGD WPEALAISQR WQQQDKAANK ELTFLLFSCP
HRLREHLERG RGNLEWKEPP SMRLKARPSS PGFSVLTCSA
FSFYPPELQL RFLRNGLAAG TGQGDFGPNS DGSFHASSSL
TVKSGDEHHY CCIVQHAGLA QPLRVELESPAKSS

The FcRn α chain sequence, showing residues involved in interaction with 1519g57 Fab′ (bold) and residues critical for interaction with Fc of IgG (underlined). All but 1 of the latter are included in the former.

### Example 2

This example describes a Phase 2, multicenter, open-label, multiple-dose, multiple-arm study to evaluate the safety, tolerability, and efficacy of UCB7665 administered as subcutaneous (sc) doses (TP0001).

### Rationale for Dose Selection

A single ascending dose study in healthy subjects (UP0018) explored the dose range of UCB7665 (between 1 and 7 mg/kg) and characterized the PK and PD effect on total IgG.

Preliminary data indicate that mean absolute decreases in IgG and mean percent change from Baseline IgG were greater in the active dose groups (n=6 each) compared to the pooled iv and sc placebo group (n=12) with maximum decreases of 49.3% (range: 44.6% to 55.9%) observed on Day 6 for a UCB7665 7 mg/kg iv dose and 42.8% (range: 39.6% to 48.6%) on Day 9 for a UCB7665 7 mg/kg sc dose.

The dose-exposure-response relationship, with total IgG as primary endpoint, was determined using non-linear mixed effects modeling. The derived population PK-PD (structural PK-PD model based on that of Lowe [Lowe et al, 2010]) was then used to guide, through simulation, the selection of appropriate repeat dose regimens that would mimic decreases achieved by plasmapheresis paradigms and result in IgG reductions of 70% or greater.

The model-based simulations demonstrate that weekly doses of UCB7665 4 mg/kg for 5 consecutive weeks are expected to produce maximum mean IgG reductions of >70%. Similar reductions are predicted to be achieved following 3 consecutive weekly doses of UCB7665 7 mg/kg.

The TP001 study is a, multicenter, open-label, multiple-dose, multiple-arm study to evaluate the safety, tolerability, and efficacy of UCB7665 administered as subcutaneous (sc) doses of 4 mg/kg, 7 mg/kg, 10 mg/kg, 15 mg/kg, and 20 mg/kg (resulting in cumulative doses of 20 mg/kg, 21 mg/kg, 20 mg/kg, 15 mg/kg and 20 mg/kg respectively), in subjects ≥18 years of age with persistent (>3 months up to 12 months after diagnosis) or chronic (more than 12 months after diagnosis) primary ITP.

A total of approximately 48 to 66 subjects (dependent on emerging safety data and corresponding DMC recommendation) are planned to enter the Dosing Period in the study. The maximum study duration for study participation for an individual subject is approximately 16 weeks.

The study is intended to evaluate 5 dose arms of UCB7665. Subjects in Dose Arm 1 will receive 5 doses of UCB7665 4 mg/kg sc at 1 week intervals, subjects in Dose Arm 2 will receive 3 doses of UCB7665 7 mg/kg sc at 1 week intervals, subjects in Dose Arm 3 will receive 2 doses of UCB7665 10 mg/kg sc at 1 week intervals, Dose Arm 4 will receive 1 dose of UCB7665 15 mg/kg sc and Dose Arm 5 will receive 1 dose of UCB7665 20 mg/kg sc. See FIG. 3.

The study consists of a Screening Period (up to 4 weeks), Dosing Period of 1 day to 4 weeks where dosing arms are introduced sequentially, and an Observation Period of 8 weeks. The Screening Visit corresponds to Visit 1 of the study. The Dosing Period will commence at Visit 2 (Baseline Visit), with dosing visits scheduled at weekly intervals for Dose Arms 1, 2 and 3 and only one visit scheduled for Dose Arms 4 and 5. The Observation Period will start after the last dose administration (or only dose administration for Dose Arms 4 and 5) with a visit scheduled 3 days after the last dosing and weekly visits thereafter (ie, weekly after last or only dose) for a period of 8 weeks. The End-of-Study Visit will be performed at the end of the Observation Period, ie, 8 weeks after the last or only dose of investigational medicinal product (IMP).

The primary objective of the study is to evaluate the safety and tolerability of UCB7665 administered by sc infusion in subjects with ITP.

The secondary objective of the study is to assess the clinical efficacy of UCB7665 as measured by the change in platelet count and to assess the pharmacodynamic (PD) effect of UCB7665 as measured by the change in total IgG concentrations in serum. The exploratory objectives include the following: to evaluate the effect of UCB7665 on ITP-specific autoantibodies to glycoproteins (GP) Ia/IIa, GPIIb/IIIa, and GPIb/IX in serum; to evaluate the clinical efficacy as measured by the change in ITP bleeding score; to evaluate the effect of UCB7665 on the concentrations of total protein, albumin, α-globulin and β-globulin, IgG subclasses, IgM, IgA, and IgE, and serum and plasma complement levels; to evaluate the emergence of anti-drug antibodies (ADA), ie, anti-UCB7665 antibodies with respect to immunogenicity and pharmacokinetics (PK)/PD; to evaluate the relationship between changes in platelet count and total IgG, IgG subclasses, ITP-specific autoantibodies; and to assess the plasma concentrations of UCB7665 administered by sc infusion.

The following efficacy variables will be assessed: Response (platelet count ≥30×10⁹/L and at least 2-fold increase of the Baseline count) during the study and by visit; Complete Response (platelet count ≥100×10⁹/L) during the study and by visit; platelet count ≥50×10⁹/L during the study and by visit; the maximum value and maximum increase from Baseline in platelet count during the study; value and change from Baseline in platelet count over time; Baseline-corrected area under the effect curve (AUEC) for platelet count; time to Response (time from starting treatment to achievement of Response); time to Complete Response (time from starting treatment to achievement of Complete Response); time to achieving platelet count ≥50×10⁹/L; duration of Response (measured from achievement of Response to loss of Response [defined as platelet count below 30×10⁹/L or less than 2-fold increase of Baseline platelet count]); duration of Complete Response (measured from achievement of Complete Response to loss of Complete Response [defined as platelet count below 100×10⁹/L]); duration of platelet count ≥50×10⁹/L (measured from achievement of platelet count ≥50×10⁹/L to reduction of platelet count below 50×10⁹/L); Clinical Response (defined as platelet count ≥30×10⁹/L and at least 2-fold increase from Baseline value and absence of bleeding); time to Clinical Response (time from starting treatment to achievement of Clinical Response); duration of Clinical Response (measured from achievement of Clinical Response to loss of Clinical Response [loss of Clinical Response defined as platelet count <30×10⁹/L or less than 2-fold increase from Baseline platelet count or presence of bleeding]); Complete Clinical Response (defined as platelet count ≥100×10⁹/L and absence of bleeding); time to Complete Clinical Response (time from starting treatment to achievement of Complete Clinical Response); duration of Complete Clinical Response (measured from achievement of Complete Clinical Response to loss of Complete Clinical Response [loss of Complete Clinical Response defined as platelet count <100×10⁹/L or presence of bleeding]); no Clinical Response (defined as platelet count <30×10⁹/L and less than 2-fold increase from Baseline or presence of bleeding); ITP bleeding score over time; and Patient Reported Outcome (PRO), ie, Neurological Fatigue Index for Multiple Sclerosis (NFI-MS) summary score over time. Plasma concentration of UCB7665 over time will be assessed as the PK variable. The PD variables are minimum value and maximum decrease in total IgG concentration during the study; IgG subclass concentrations; and ITP-specific autoantibody (GPIa/IIa, GPIIb/IIIa, and GPIb/IX) in serum over time.

### Assessment of Efficacy

### Platelet Counts

For assessment of platelet counts, blood samples will be collected by qualified site personnel at

the same time that samples are collected for standard clinical laboratory assessments.

Platelet counts will be determined by a central laboratory and the following variables will be computed in the statistical database for the purpose of analysis:
Response by visit and Response at least once during the study
Complete Response by visit and Complete Response at least once during the study
Platelet count ≥50×109/L by visit and at least once during the study
Value and change from Baseline in platelet count by visit
Baseline-corrected AUEC for platelet count calculated from Baseline to the end of study visit
Maximum value and maximum increase from Baseline
Time to Response
Time to Complete Response
Time to achieving platelet count ≥50×109/L
Duration of Response
Duration of Complete Response
Duration of platelet count ≥50×109/L
Clinical Response: platelet count ≥30×109/L and at least 2-fold increase from Baseline value
   and absence of bleeding
Time to Clinical Response: time from starting treatment to achievement of Clinical Response
Duration of Clinical Response: measured from achievement of Clinical Response to loss of Clinical Response (loss of Clinical Response defined as platelet count <30×109/L or less than 2-fold increase from Baseline platelet count or presence of bleeding)
Complete Clinical Response: platelet count ≥100×109/L and absence of bleeding
Time to Complete Clinical Response: time from starting treatment to achievement of Complete Clinical Response
Duration of Complete Clinical Response: measured from achievement of Complete Clinical
   Response to loss of Complete Clinical Response (loss of Complete Clinical Response defined as platelet count <100×109/L or presence of bleeding)
No Clinical Response: platelet count <30×109/L or less than 2-fold increase from Baseline or
   presence of bleeding

The clinical response variables will be assessed only for visits for which both platelet counts and

the ITP bleeding score are assessed (with the exception of the confirmatory platelet assessments
which may be obtained at any visit [scheduled or unscheduled] provided that they meet the criteria below). In order to define a clinical response, the platelet count must be confirmed on 2
separate occasions at least 7 days apart (ie, the second assessment should be ≥168 hours after the
first assessment). The time to response will be taken as the time to the first platelet assessment
(obtained at the same time as the corresponding ITP bleeding score assessment). If the second assessment does not fulfill the required criteria for a clinical response, the subject will be considered as a nonresponder at the respective visits. In order to define a clinical response (or no
clinical response), the platelet count must be confirmed on 2 separate occasions. Absence of bleeding is indicated by Grade 0 for all domains of the SMOG. Presence of bleeding is indicated by a Grade of 1 or above, for at least one domain of the SMOG.

### ITP Bleeding Score

The International Working Group on ITP now proposes a consensus-based ITP-specific Bleeding

Assessment Tool (ITP-BAT), based on a precise definition of bleeding manifestations and on the

grading of their severity (Rodeghiero et al, 2013, Standardization of bleeding assessment in immune thrombocytopenia: report from the International Working Group. Blood.
121(14):2596-606.).

The ITP bleeding score will be assessed using the ITP-BAT tool version 1.0. Assessment will be performed according to the schedule of study assessments.

For the ITP-BAT, bleeding manifestations were grouped into 3 major domains: skin (S), visible

mucosae (M), and organs (0), with gradation of severity (SMOG). Each bleeding manifestation
is assessed at the time of examination. Severity is graded from 0 to 3 or 4, with grade 5 for any
fatal bleeding. Bleeding reported by the subject without medical documentation is graded 1. Within each domain, the same grade is assigned to bleeding manifestations of similar clinical impact. The “worst” bleeding manifestation since the last Observation Period visit is graded, and
the highest grade within each domain is recorded. The SMOG system provides a consistent description of the bleeding phenotype in ITP.

A standardized data collection form will be used to facilitate collection of information and communication among physicians and investigators. The grading of bleeding symptoms at presentation and at each subsequent evaluation is presented in FIG. 4.

Interim Results of Study TP0001 (with Rozanolixizumab—UCB7665) in Patients with Immune Thrombocytopenia

Outcomes of Study TP0001 interim data (The study is currently ongoing with 10, 15 and 20 mg/kg doses).

The study initially tested 2 doses; 4 mg/kg and 7 mg/kg cohorts, 15 patients per cohort (30 patients in total). These patients have completed the study. Based on the outcome of these doses additional doses of (10 mg/kg, two doses), 15 mg/kg (single dose), and 20 mg/kg (single dose) are planned. To date, 6 patients have been successfully dosed at 10 mg/kg.

### Interim Results:

Data obtained to date from patients in the originally designed study (4 mg/kg and 7 mg/kg dose cohorts, who have completed the study) indicates a positive outcome for this phase 2a study regarding safety and tolerability and positive signal regarding efficacy.

### Safety and Tolerability

UCB7665 was well tolerated following multiple doses; no treatment discontinuations due to TEAEs or TEAEs leading to death were reported.

### Pharmacodynamics

At the interim analysis, maximum mean decreases in total IgG levels were observed at Day 29 for the 4 mg/kg group (43.6%, range 21.9-68.6) and at Day 22 for the 7 mg/kg group (50.5%, 35.7-65.5) (FIG. 5).

Data to date indicates maximum mean decreases in total IgG levels were observed at Day 15 for the 10 mg/kg group (FIG. 5), study on going.

### Efficacy (Platelet Response)

Clinically relevant improvements in platelet counts were observed, using different response criteria (see table below and FIG. 6 which shows mean platelet count for responders only). The response criterion 1 platelet count ≥30×10⁹/L and at least 2-fold increase of the baseline value is derived from the EMA guideline 2014 (Guideline on the clinical development of medicinal products intended for the treatment of chronic primary immune thrombocytopenia, 20 Feb. 2014 EMA/CHMP/153191/2013 Oncology Working Party), response criterion 2 (platelet count ≥50×10⁹/L during the study) is widely accepted as clinically meaningful response and also accepted response criterion for registration studies for ITP at FDA. Response criterion 3 (platelet count ≥100×10⁹/L) is derived from the “complete response” definition in the EMA guideline for ITP from 2014. Based on these interim results, UCB7665 has modulated clinically and regulatory relevant variables. Responders who received confounding rescue medication in the study (such as IVIg or TPOs) were deducted from the assessment.

| | 15 patients total | 15 patients total |
|---|---|---|
| Platelet response | 4 mg/kg (n/%) | 7 mg/kg (n/%) |
| 1. Response criterion: | 7 (46.7) | 8 (53.3) |
| count ≥ 30 × 10⁹/L and at least | | |
| 2-fold increase of the baseline value | | |
| 2. Response criterion: | 5 (33.3) | 5 (33.3) |
| count ≥ 50 × 10⁹/L during the study | | |
| 3. Response criterion: | 4 (26.7) | 2 (13.3) |
| Complete response: platelet | | |
| count ≥ 100 × 10⁹/L | | |

FIG. 6 shows mean platelet count (for responders only) for 4 mg/kg, 7 mg/kg and 10 mg/kg doses (10 mg/kg study ongoing). The preliminary 10 mg/kg data obtained to date, suggests that for responders, the mean platelet count is higher than for responders at the 4 and 7 mg/kg doses.

The study is ongoing and higher doses are currently being evaluated: (10 mg/kg, two doses), 15 mg/kg (single dose), & 20 mg/kg (single dose).

Efficacy Platelet Response (10 mg/Kg Update)

Efficacy data for the 10 mg/kg (two doses) is shown in the table below. Clinically relevant improvements in platelet counts were observed, using different response criteria.

| | 12 patients total |
|---|---|
| Platelet response | 10 mg/kg (n/%) |
| 1. Response criterion: | 6 (50) |
| count ≥ 30 × 10⁹/L and at least | |
| 2-fold increase of the baseline | |
| value | |
| 2. Response criterion: | 6 (50) |
| count ≥ 50 × 10⁹/L during the | |
| study | |
| 3. Response criterion: | 4 (33.3) |
| Complete response: platelet | |
| count ≥ 100 × 10⁹/L | |

The study is ongoing and higher doses are currently being evaluated: 15 mg/kg (single dose), & 20 mg/kg (single dose).

FIG. 7 shows the decrease from baseline (mean IgG reduction) following multiple UCB7665 4, 7, 10 and single 15 mg/kg administration. Data to date indicate maximum mean decreases in total IgG levels were observed at Day 8 for the 15 mg/kg group (12 patients) (FIG. 7).

The preliminary 15 mg/kg data obtained to date, suggests that for responders, the platelet responses are at least comparable to those observed for 10 mg/kg.

1. A method of treating or preventing immune (idiopathic) thrombocytopenic purpura (ITP) in a human in need thereof, the method comprising administering to the human in the range of 1 to 5 doses of an anti-FcRn antibody or antigen binding fragment thereof over a treatment period of 1 to 12 weeks, wherein the aggregrate dose in the treatment period is in the range of 1 to 30 mg per kg.

2. An anti-FcRn antibody or binding fragment thereof for use in the treatment or prevention of immune (idiopathic) thrombocytopenic purpura (ITP) in a human in need thereof, comprising administering to the human in the range of 1 to 5 doses of the antibody or antigen binding fragment thereof over a treatment period of 1 to 12 weeks, wherein the aggregrate dose in the treatment period is in the range of 1 to 30 mg per kg.

3. Use of an anti-FcRn antibody or antigen binding fragment thereof for the manufacture of a medicament for the treatment or prevention of immune (idiopathic) thrombocytopenic purpura (ITP), comprising administering in the range of 1 to 5 doses of the antibody or binding fragment thereof over a treatment period of 1 to 12 weeks, wherein the aggregrate dose in the treatment period is in the range of 1 to 30 mg per kg

4. A method, an anti-FcRn antibody or antigen binding fragment thereof or a use according to any one of claims 1 to 3, wherein the antibody or binding fragment thereof comprises:
a. a heavy chain or heavy chain fragment having a variable region, wherein said variable region comprises three CDRs having the sequences given in SEQ ID NO: 1 for CDR H1, SEQ ID NO: 2 for CDR H2 and SEQ ID NO: 3 for CDR H3, and
b. a light chain or light chain fragment thereof having a variable region, wherein said variable region comprises three CDRs having the sequences given in SEQ ID NO: 4 for CDR L1, SEQ ID NO: 5 for CDR L2 and SEQ ID NO: 6 for CDR L3,

5. A method, an anti-FcRn antibody or binding fragment thereof or a use according to any one of claims 1 to 4, wherein the antibody or antigen binding fragment thereof is humanized.

6. A method, an anti-FcRn antibody or antigen binding fragment thereof or a use according to any one of claims 1 to 5 wherein the anti-FcRn antibody or binding fragment thereof comprises a heavy chain comprising the sequence given in SEQ ID NO:29 or a sequence specific to FcRn at least 80% identical thereto.

7. A method, an anti-FcRn antibody or antigen binding fragment thereof or a use according to any one of claims 1 to 6 wherein the anti-FcRn antibody or binding fragment thereof comprises a light chain comprising the sequence given in SEQ ID NO:15 or a sequence specific to FcRn at least 80% identical thereto.

8. A method, an anti-FcRn antibody or antigen binding fragment thereof or a use according to claim 4 or 5 wherein the anti-FcRn antibody or binding fragment thereof comprises a heavy chain variable domain sequence having the sequence given in SEQ ID NO:29 and a light chain variable domain sequence comprising the sequence given in SEQ ID NO:15.

9. A method, an anti-FcRn antibody or antigen binding fragment thereof or a use according to any one of claims 1 to 8, wherein the antibody binding fragment is a scFv, Fv, Fab or Fab′fragment.

10. A method, an anti-FcRn antibody or antigen binding fragment thereof or a use according to claim 9 wherein the anti-FcRn antibody or binding fragment thereof comprises a heavy chain comprising the sequence given in SEQ ID NO:36 and a light chain comprising the sequence given in SEQ ID NO:22.

11. A method, an anti-FcRn antibody or a use according to any one of claims 1 to 7, wherein the antibody is a full length antibody.

12. A method, an anti-FcRn antibody or a use according to claim 11 wherein the full length antibody is selected from the group consisting of an IgG1, IgG4 and IgG4P.

13. A method, an anti-FcRn antibody or a use according to claim 11 or claim 12 wherein the anti-FcRn antibody has a heavy chain comprising the sequence given in SEQ ID NO:72 or SEQ ID NO:87 or SEQ ID NO:43 and a light chain comprising the sequence given in SEQ ID NO:22.

14. A method, an anti-FcRn antibody or a use according to claim 11, 12 or 13 wherein the anti-FcRn antibody is UCB7665 (rozanolixizumab).

15. A method, an anti-FcRn antibody or antigen binding fragment thereof or a use to any one of claims 1 to 8, wherein the antibody or binding fragment thereof is a Fab-dsFv having a heavy chain comprising the sequence given in SEQ ID NO:50 and a light chain comprising the sequence given in SEQ ID NO:46 or SEQ ID NO:78.

16. A method, an anti-FcRn antibody or antigen binding fragment thereof or a use according to any one of claims 1 to 15 having a binding affinity for human FcRn of 100 pM or less.

17. A method, an anti-FcRn antibody or antigen binding fragment thereof or a use according to claim 16 wherein the binding affinity for human FcRn is 100 pM or less when measured at pH6 and at pH7.4.

18. A method, an anti-FcRn antibody or antigen binding fragment thereof or a use according to any one of claims 1 to 17 wherein the antibody or antigen binding fragment is provided as a pharmaceutical composition comprising one or more of a pharmaceutically acceptable excipient, diluent or carrier.

19. A method, an anti-FcRn antibody or antigen binding fragment thereof or a use according to claim 18, wherein the pharmaceutical composition further comprises one or more other active ingredients.

20. A method, an anti-FcRn antibody or antigen binding fragment thereof or a use according to any one of claims 1 to 19, wherein the treatment is period is 1, 2, 3, 4, 5, 6, 7 or 8 weeks.

21. A method, an anti-FcRn antibody or antigen binding fragment thereof or a use according to claim 20, wherein the treatment is period is 1, 2, 3, 4 or 5 weeks.

22. A method, an anti-FcRn antibody or antigen binding fragment thereof or a use according to any one of claims 1 to 21, wherein 1, 2, 3, 4 or 5 doses of antibody or binding fragment are administered and each dose is in the range of 4 mg/kg to 30 mg/kg.

23. A method, an anti-FcRn antibody or antigen binding fragment thereof or a use according to claim 22, wherein each dose is 4 mg/Kg, for example administered as five individual doses, in particular over a treatment period of five weeks, in particular five consecutive weeks.

24. A method, an anti-FcRn antibody or antigen binding fragment thereof or a use according to claim 22, wherein each dose is 7 mg/Kg, for example administered as three individual doses, in particular over a treatment period of three weeks, in particular three consecutive weeks.

25. A method, an anti-FcRn antibody or antigen binding fragment thereof or a use according to claim 22, wherein each dose is 10 mg/Kg, for example administered as two individual doses, in particular over a treatment period of two weeks, in particular two consecutive weeks.

26. A method, an anti-FcRn antibody or antigen binding fragment thereof or a use according to claim 22, wherein a single dose is administered.

27. A method, an anti-FcRn antibody or antigen binding fragment thereof or a use according to claim 26 wherein the dose is 15 mg/Kg.

28. A method, an anti-FcRn antibody or antigen binding fragment thereof or a use according to claim 26, wherein the dose is 20 mg/Kg.

29. A method, an anti-FcRn antibody or antigen binding fragment thereof or a use according to claim 26, wherein the dose is 25 mg/Kg.

30. A method, an anti-FcRn antibody or antigen binding fragment thereof or a use according to claim 26, wherein the dose is 30 mg/Kg.

31. A method, an anti-FcRn antibody or antigen binding fragment thereof or a use according to any one of claims 1 to 30 wherein the aggregate dose is selected from 10, 15, 20, 21, 25 and 30 mg/Kg.

32. A method, an anti-FcRn antibody or antigen binding fragment thereof or a use according to any one of claims 1 to 31 wherein the anti-FcRn antibody or binding fragment thereof is administered subcutaneously or intravenously.

33. A method, an anti-FcRn antibody or antigen binding fragment thereof or a use according to any one of claims 1 to 32 wherein the an anti-FcRn antibody or antigen binding fragment thereof blocks binding of human IgG to human FcRn.

34. A method, an anti-FcRn antibody or antigen binding fragment thereof or a use according to any one of claims 1 to 33 wherein the an anti-FcRn antibody or antigen binding fragment thereof does not bind β2 microglobulin.

35. A method, an anti-FcRn antibody or antigen binding fragment thereof or a use according to any one of claims 1 to 34 wherein the method further comprises administering one or more additional doses that are lower than the initial dose.

36. A method, an anti-FcRn antibody or antigen binding fragment thereof or a use according to any one of claims 1-34 wherein the anti-FcRn antibody or antigen binding fragment thereof binds to an epitope of human FcRn which comprises at least one amino acid selected from the group consisting of residues V105, P106, T107, A108 and K109 of SEQ ID NO:94 and at least one residue, for example at least 2, 3, 4, 5, 6, 7, 8, 9 or 10 residues selected from the group consisting of P100, E115, E116, F117, M118, N119, F120, D121, L122, K123, Q124, G128, G129, D130, W131, P132 and E133 of SEQ ID NO:94.
